(19) United States
(12) Patent Application Publication
(10) Pub. No.: US 2007/0167428 A1
(43) Pub. Date: Jul. 19, 2007

(54) Thienopyrroles as antagonists of gnrh

(75) Inventors: Jean-Claude Arnould, Reims (FR); Craig Steven Harris, Reims (FR); Paul Jones, Montreal (CA)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 10/598,118

(22) PCT Filed: Feb. 17, 2005
(86) PCT No.: PCT/GB05/00568

§ 371 Date: Oct. 3, 2006

(30) Foreign Application Priority Data
Feb. 20, 2004 (EP) 04290466.4

(51) Int. Cl. A61K 31/5377; A61K 31/496; A61K 31/454; A61K 31/407; C07D 498/02

(52) U.S. Cl. 514/210.21; 514/412; 514/320; 514/253.09; 514/254.08; 514/232.8; 548/453; 544/143; 544/373; 546/198

(57) ABSTRACT

The invention relates to a group of novel thieno-pyrrole compounds of Formula (I) wherein: R¹, R², R³, R⁴ and R⁵ are as defined in the specification, which are useful as gonadotrophin releasing hormone antagonists. The invention also relates to pharmaceutical formulations of said compounds, methods of treatment using said compounds and to processes for the preparation of said compounds.

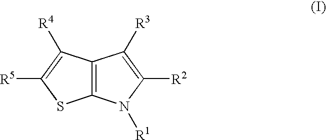

The present invention relates to compounds which are antagonists or gonadotropin releasing hormone (GnRH) activity. The invention also relates to pharmaceutical formulations, the use of a compound of the present invention in the manufacture of a medicament, a method of therapeutic treatment using such a compound and processes for producing the compounds.

Gonadotropin releasing hormone (GnRH) is a decapeptide that is secreted by the hypothalamus into the hypophyseal portal circulation in response to neural and/or chemical stimuli, causing the biosynthesis and release of luteinizing hormone (LH) and follicle-stimulating hormone (FSH) by the pituitary. GnRH is also known by other names, including gonadoliberin, LH releasing hormone (LHRH), FSH releasing hormone (FSH RH) and LH/FSH releasing factor (LH/FSH RF).

GnRH plays an important role in regulating the action of LH and FSH (by regulation of their levels), and thus has a role in regulating the levels of gonadal steroids in both sexes, including the sex hormones progesterone, oestrogens and androgens. More discussion of GnRH can be found in WO 98/55119 and WO 97/14697, the disclosures of which are incorporated herein by reference.

It is believed that several diseases would benefit from the regulation of GnRH activity, in particular by antagonising such activity. These include sex hormone related conditions such as sex hormone dependent cancer, benign prostatic hypertrophy and myoma of the uterus. Examples of sex hormone dependent cancers are prostatic cancer, uterine cancer, breast cancer and pituitary gonadotrophe adenoma.

The following disclose compounds purported to act as GnRH antagonists: WO 97/21435, WO 97/21703, WO 97/21704, WO 97/21707, WO 55116, WO 98/55119, WO 98/55123, WO 98/55470, WO 98/55479, WO 99/21553, WO 99/21557, WO 99/41251, WO 99/41252, WO 00/04013, WO 00/69433, WO 99/51231, WO 99/51232, WO 99/51233, WO 99/51234, WO 99/51595, WO 99/51596, WO 00/53178, WO 00/53180, WO 00/53179, WO 00/53181, WO 00/53185, WO 00/53602, WO 02/066477, WO 02/066478, WO 02/06645 and WO 02/092565. In addition, co-pending WO2004/018480 and WO2004/018479, which were unpublished at the date of the present application, describe a range of thienopyrrole derivatives which have this activity.

It would be desirable to provide further compounds, such compounds being GnRH antagonists. Thus, according to the first aspect of the invention there is provided a compound of Formula (I), 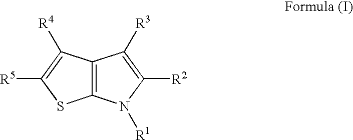
wherein:
R¹ is selected from: hydrogen, optionally substituted C₁₋₆alkyl, optionally substituted aryl or optionally substituted arylC₁₋₆alkyl, wherein the optional substituents are selected from C₁₋₄alkyl, nitro, cyano, fluoro and C₁₋₄alkoxy;
R² is hydrogen, optionally substituted C₁₋₆alkyl or an optionally substituted mono or bi-cyclic aromatic ring, wherein the optional substituents are 1, 2 or 3 substituents independently selected from: cyano, R^{c}R^{f}N—, C₁₋₆alkyl, C₁₋₆alkoxy, halo, haloC₁₋₆alkyl or haloC₁₋₆alkoxy wherein R^{e} and R^{f} are independently selected from hydrogen, C₁₋₆alkyl or aryl;
R³ is selected from a group of Formula (IIa) to Formula (IId): 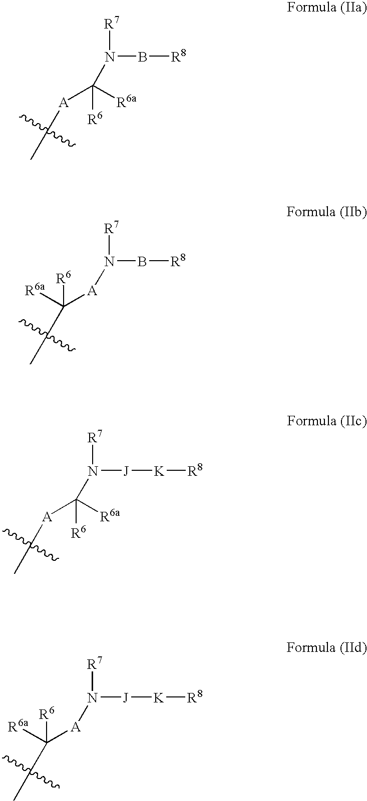
R⁴ is selected from hydrogen, C₁₋₄alkyl or halo;
R⁵ is selected from a group of Formula III-a; III-b; III-c; III-d; III-e; III-f, III-g, III-h, III-i, or III-j, III-k, III-I, III-m, III-n, III-o or III-p 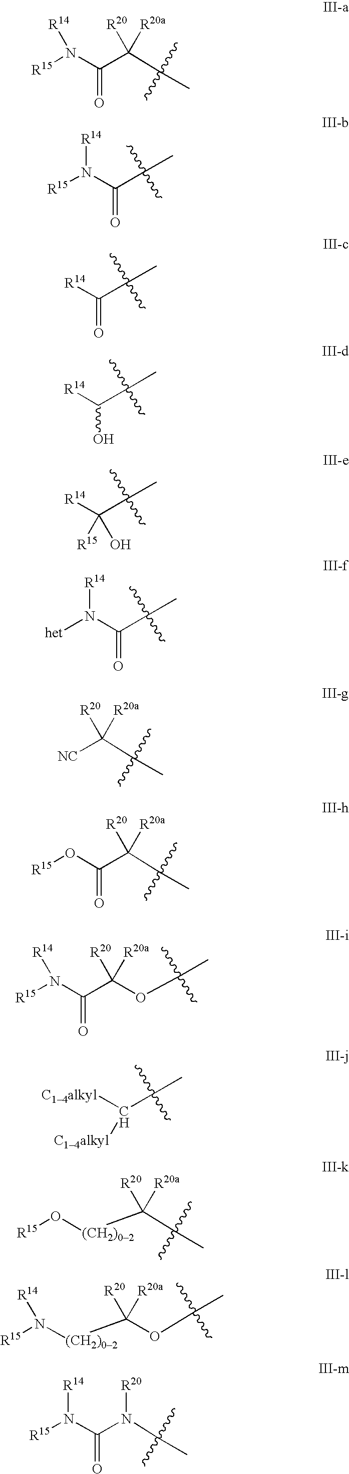 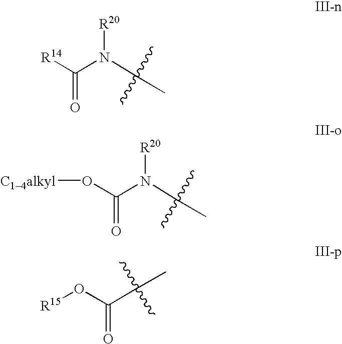
   wherein:
   het represents an optionally substituted 3- to 8-membered heterocyclic ring containing from 1 to 4 heteroatoms independently selected from O, N and S, wherein the optional substituents are selected from 1-2 groups selected from R¹² and R¹³;
R¹⁴ and R¹⁵ are selected from:
   (i) R¹⁴ is selected from hydrogen; optionally substituted C₁₋₈alkyl; optionally substituted aryl; —R^{d}—Ar, where R^{d} represents C₁₋₈alkylene and Ar represents optionally substituted aryl; and optionally substituted 3- to 8-membered heterocyclic ring optionally containing from 1 to 3 further heteroatoms independently selected from O, N and S; and R¹⁵ is selected from hydrogen; optionally substituted C₁₋₈alkyl and optionally substituted aryl;
   (ii) when R⁵ represents a group of Formula III-a , III-b, III-i, III-l or III-m, then the group NR¹⁴(—R¹⁵) additionally represents an optionally substituted 3- to 8-membered heterocyclic ring optionally containing from 1 to 3 further heteroatoms independently selected from O, N and S; or
   (iii) when R⁵ represents structure III-e, 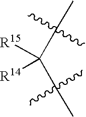
      represents an optionally substituted 3- to 8-membered heterocyclic ring optionally containing from 1 to 4 heteroatoms independently selected from O, N and S;
R²⁰ and R^{20a} are independently selected from hydrogen, fluoro or optionally substituted C₁₋₈alkyl, or R²⁰ and R^{20a} together with the carbon atom to which they are attached form an optionally substituted 3 to 7-membered cycloalkyl ring;
R⁶ and R^{6a} are independently selected from hydrogen, fluoro, optionally substituted C₁₋₆alkyl, C₁₋₆alkoxy, N-C₁₋₆alkylamino and N,N-diC₁₋₆alkylamino or R⁶ and R^{6a} taken together and the carbon atom to which they are attached form a carbocyclic ring of 3-7 atoms or R⁶ and R^{6a} taken together and the carbon atom to which they are attached form a carbonyl group;
or when A is not a direct bond, the group 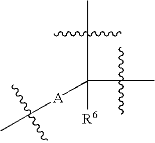
   forms a carbocyclic ring of 3-7 carbon atoms or a heterocyclic ring containing one or more heteroatoms;
or the group 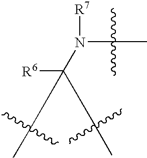
   forms a heterocyclic ring containing 3-7 carbon atoms and one or more heteroatoms;
R⁷ is selected from: hydrogen or C₁₋₆alkyl;
R⁸ is selected from:
   (i) hydrogen, C₁₋₆alkyl, C₂₋₆alkenyl, C₂₋₆alkynyl, haloC₁₋₆alkyl, C₁₋₄-alkoxyC₁₋₄alkyl, hydroxy, hydroxyC₁₋₆alkyl, cyano, N-C₁₋₄alkylamino, N,N-di-C₁₋₄alkylamino, C₁₋₆alkyl-S(O₁)—, —O—R^{b}, —NR^{b}R^{c}, —C(O)—R^{b}, —C(O)O—R^{b}, —CONR^{b}R^{c}, NH—C(O)—R^{b} or —S(Oₙ)NR^{b}R^{c},
      where R^{b} and R^{c} are independently selected from hydrogen and C₁₋₆alkyl (e.g. C₁₋₄alkyl) optionally substituted with hydroxy, amino, N-C₁₋₄alkylamino, N,N-di-C₁₋₄alkylamino, HO—C₂₋₄alkyl-NH— or HO—C₂₋₄alkyl-N(C₁₋₄alkyl)-;
   (ii) nitro when B is a group of Formula (IV) and X is CH and p is 0;
   (iii) carbocyclyl (such as C₃₋₇cycloalkyl or aryl) or arylC₁₋₆alkyl each of which is optionally substituted by R¹², or R¹³;
   (iv) heterocyclyl or heterocyclylC₁₋₆alkyl each of which is optionally substituted by up to 4 substituents independently selected from R¹² or R¹³ and where any nitrogen atoms within a heterocyclyl group are, where chemically allowed, optionally in their oxidised (N→O, N—OH) state;
R¹² is independently selected from: halo, hydroxy, hydroxyC₁₋₆alkyl, oxo, cyano, cyanoC₁₋₆alkyl, nitro, carboxyl, C₁₋₆alkyl, C₁₋₆alkoxy, C₁₋₆alkoxyC₁₋₄alkyl, C₁₋₆alkoxycarbonylC₀₋₄alkyl, C₁₋₆alkanoylC₀₋₄alkyl, C₁₋₆alkanoyloxyC₀₋₄alkyl, C₂₋₆alkenyl, C₁₋₃perfluoroalkyl-, C₁₋₃perfluoroalkoxy, aryl, arylC₁₋₆alkyl, heterocyclyl, heterocyclylC₁₋₆alkyl, aminoC₀₋₄alkyl, N-C₁₋₄alkylaminoC₀₋₄alkyl, N,N-di-C₁₋₄alkylaminoC₀₋₄alkyl, carbamoyl, N-C₁₋₄alkylcarbamoylC₀₋₂alkyl, N,N-di-C₁₋₄alkylaminocarbamoylC₀₋₂alkyl, aminocarbonylC₀₋₄alkyl, N-C₁₋₆alkyaminocarbonylC₀₋₄alkyl, N,N-C₁₋₆alkyaminocarbonylC₀₋₄alkyl, C₁₋₆alkyl-S(O)ₙ-aminoC₀₋₄alkyl-, aryl-S(O)ₙ-aminoC₀₋₂alkyl-, C₁₋₃perfluoroalkyl-S(O)ₙ-aminoC₀₋₂alkyl-; C₁₋₆alkylamino-S(O)ₙ—CO₀₋₂alkyl-, arylamino-S(O)ₙ—C₀₋₂alkyl-, C₁₋₃perfluoroalkylamino-S(O)ₙ—C₀₋₂alkyl-, C₁₋₆alkanoylamino-S(O)ₙ-C₀₋₂alkyl-; arylcarbonylamino-S(O)ₙ—C₀₋₂alkyl-, C₁₋₆alkyl-S(O)ₙ—C₀₋₂alkyl-, aryl-S(O)ₙ—C₀₋₂alkyl-, C₁₋₃perfluoroalkyl-, C₁₋₃perfluoroalkoxyC₀₋₂alkyl; R^{9′}OC(O)(CH₂)_{w}—, R^{9″}R^{10″}N(CH₂)_{w}—, R^{9′}R^{10′}NC(O)(CH₂)_{w}—, R⁹R¹⁰NC(O)N(R⁹)(CH₂)_{w}—, R⁹OC(O)N(R⁹)(CH₂)_{w}—, or halo, wherein w is an integer between 0 and 4 and R⁹ and R¹⁰ are independently selected from hydrogen, C₁₋₄alkyl, C₁₋₄alkylsulphonyl and C₃₋₇carbocyclyl, R^{9′} and R^{10′} are independently selected from C₁₋₄alkylsulphonyl and C₃₋₇carbocyclyl, and R^{9″} and R^{10 ″} are C₃₋₇carbocyclyl; wherein an amino or an aryl group within R¹² is optionally substituted by C₁₋₄alkyl;
R¹³ is —C(O)—R¹⁶;
R¹⁶ is selected from an amino acid derivative or an amide of an amino acid derivative;
R¹⁷ is hydrogen or C₁₋₄alkyl;
A is selected from:
   (i) a direct bond;
   (ii) optionally substituted C₁₋₅alkylene wherein the optional substituents are independently selected from: hydroxy, hydroxyC₁₋₆alkyl, C₁₋₆alkyl, C₁₋₆alkoxy, C₁₋₄alkoxyC₁₋₄alkyl, aryl, arylC₁₋₆alkyl;
   (iii) a carbocyclic ring of 3-7 atoms;
   (iv) a carbonyl group or —C(O)—C(R^{d}R^{d})—, wherein each R^{d} is independently selected from hydrogen and C₁₋₂alkyl;
or when R³ is a group of Formula (IIa) or (IIb), the group 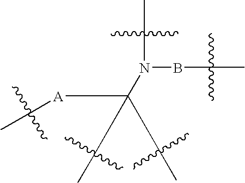
   forms a heterocyclic ring containing 3-7 carbon atoms and one or more heteroatoms;
or when R³ is a group of Formula (IIa), (IIb), (IIc) or (IId), the group 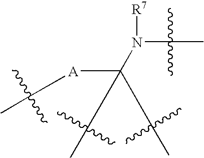
   forms a heterocyclic ring containing 3-7 carbon atoms and one or more heteroatoms;
B is selected from:
   (i) a direct bond;
   (ii) a group of Formula (IV) 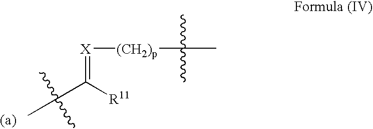
      wherein:
X is selected from N or CH,
   wherein at position (a) Formula (IV) is attached to the nitrogen atom and the (CH₂)ₚ group is attached to R⁸, and wherein R¹¹ is selected from hydrogen, optionally substituted C₁₋₆alkyl or N(R²³R²⁴),
   where R²³ and R²⁴ are independently selected from: hydrogen, hydroxy, optionally substituted C₁₋₆alkyl, optionally substituted aryl, optionally substituted arylC₁₋₆alkyl, an optionally substituted carbocyclic ring of 3-7 atoms, optionally substituted heterocyclyl, optionally substituted heterocyclylC₁₋₆alkyl or R²³ and R²⁴ taken together can form an optionally substituted ring of 3-9 atoms, wherein the optional substituents for any optionally substituted group R²³, R²⁴ and C₁₋₆alkyl groups R¹¹ are selected from R¹² and 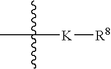
      where K and R⁸ are as defined herein;
   (iii) a group independently selected from: optionally substituted C₁₋₆alkylene, optionally substituted C₃₋₇cycloalkyl, optionally substituted C₃₋₆alkenylene, optionally substituted C₃₋₆alkynyl, (C₁₋₅alkyl)ₐₐ-S(Oₙ)—(C₁₋₅alkyl)_{bb}-, —(C₁₋₅alkyl)ₐₐ-O—(C₁₋₅alkyl)_{bb}-, —(C₁₋₅alkyl)ₐₐ-C(O)—(C₁₋₅alkyl)_{bb}- or (C₁₋₅alkyl)ₐₐ-N(R^{14a})—(C₁₋₅alkyl)_{bb}, or —(C₁₋₅alkyl)ₐₐ-C(O)NR^{14a}—(C₁₋₅alkyl)_{bb}- wherein R^{14a} is a group R¹⁴ as defined above, or R^{14a} and the (C₁₋₅alkyl)ₐₐ or (C₁₋₅alkyl)_{bb} chain can be joined to form a heterocyclic ring, wherein aa and bb are 0 or 1, and the combined length of (C₁₋₅alkyl)ₐₐ and (C₁₋₅alkyl)_{bb} is less than or equal to C₅alkyl and wherein the optional substituents are independently selected from R¹²;
or the group —B—R⁸ represents a group of Formula (V) 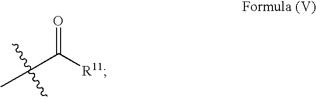
or the group 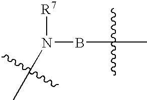
   together forms an optionally substituted heterocyclic ring containing 4-7 carbons atoms, wherein the optional substituents are selected from 1 or 2 substituents independently selected from R¹² and R¹³;
or the group 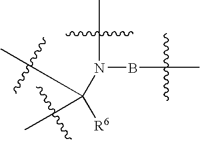
   forms a heterocyclic ring containing 3-7 carbon atoms and one or more heteroatoms;
J is a group of the formula: —(CH₂)ₛ-L-(CH₂)ₛ— or —(CH₂)ₛ—C(O)—(CH₂)ₛ-L-(CH₂)ₛ— wherein when s is greater than 0, the alkylene group is optionally substituted by 1 to 2 group selected from R¹²,
or the group 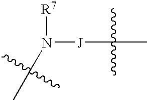
   together forms an optionally substituted heterocyclic ring containing 4-7 carbons atoms, wherein the optional substituents are selected from 1 or 2 substituents independently selected from R¹² and R¹³;
K is selected from: a direct bond, —(CR²¹R²²)ₛ₁—, —(CR²¹R²²)ₛ₁—O—(CR²¹R²²)ₛ₂—, —(CR²¹R²²)ₛ₁—(O—(CR²¹R²²)ₛ₂—, —(CR²¹R²²)ₛ₁—S(O)ₙ—(CR²¹R²²)ₛ₂—, —(CR²¹R²²)ₛ₁—N(R¹⁷)—(CR²¹R²²)ₛ₂—, —(CR²¹R²²)ₛ₁—C(O)N(R¹⁷)—(CR²¹R²²)ₛ₂—, —(CR²¹R²²)ₛ₁—N(R¹⁷)C(O)—(CR²¹R²²)ₛ₂—, —(CR²¹R²²)ₛ₁—N(R¹⁷)C(O)N(R¹⁷)—(CR²¹R²²)ₛ₂—, —(CR²¹R²²)ₛ₁—OC(O)—(CR²¹R²²)ₛ₂—, —(CR²¹R²²)ₛ₁—C(O)O—(CR²¹R²²)ₛ₂—, —(CR²¹R²²)ₛ₁—N(R¹⁷)C(O)O—(CR²¹R²²)ₛ₂, —(CR²¹R²²)ₛ₁—OC(O)N(R¹⁷)—(CR²¹R²²)ₛ₂—, —(CR²¹R²²)ₛ₁—OS(Oₙ)—)—(CR²¹R²²)ₛ₂, or —(CR²¹R²²)ₛ₁—S(Oₙ)—O—(CR²¹R²²)ₛ₂—, —(CR²¹R²²)ₛ₁—S(O)₂N(R¹⁷)—(CR²¹R²²)ₛ₂— or —(CR²¹R²²)ₛ₁—N(R¹⁷)S(O)₂—(CR²¹R²²)ₛ₂—; wherein each R²¹ and R²² group is independently selected from hydrogen, hydroxy or optionally substituted C₁₋₄alkyl, wherein the optional substitutent is a group ZR³⁰ where Z is oxygen or a group S(O)ₙ where n is as described above, and R³⁰ is hydrogen or C₁₋₄alkyl;
L is selected from optionally substituted aryl or optionally substituted heterocyclyl;
n is an integer from 0 to 2;
p is an integer from 0 to 4;
s, s1 and s2 are independently selected from an integer from 0 to 4, and s1+s2 is less than or equal to 4;
   with the proviso that the compound must contain at least one of the following groups:
(i) R³ is a group of formula (IIc) or (IId) wherein J is a group of the formula: —(CH₂)ₛ—C(O)—(CH₂)ₛ-L-(CH₂)ₛ—, and/or
(ii) R² is selected from hydrogen or optionally substituted C₁₋₆alkyl, and/or
(iii) at least one group R⁶ or R^{6a} is selected from C₁₋₆alkoxy, N-C₁₋₆alkylamino and N,N-diC₁₋₆alkylamino, and/or
(iv) R⁵ is a group of formula III-k, III-l, III-o or III-p, and/or
(v) R⁸ is selected from substituted C₃₋₇cycloalkyl, substituted aryl or substituted arylC₁₋₆alkyl, wherein a substituent is a group R¹³; or R⁸ is a heterocyclyl or heterocyclylC₁₋₆alkyl each of which is substituted by a group R¹³ and optionally by up to 3 further substituents independently selected from R¹² or R¹³; and/or
(vi) both of R²¹ and R²² within a —(CR²¹R²²)ₛ₁— or —(CR²¹R²²)ₛ₂— are C₁₋₄alkyl; or
(vii) at least one of R²¹ or R²² within a —(CR²¹R²²)ₛ₁— or —(CR²¹R²²)ₛ₂— is a C₁₋₄alkyl which is optionally substituted by a group ZR³⁰,
   or a salt, solvate or pro-drug thereof.

In a particular embodiment, the compound of formula (I) contains at least one of the following groups:
(i) R³ is a group of formula (IIc) or (IId) wherein J is a group of the formula: —(CH₂)ₛ—C(O)—(CH₂)ₛ-L-(CH₂)ₛ—, and/or
(ii) R² is selected from hydrogen or optionally substituted C₁₋₆alkyl, and/or
(iii) at least one group R⁶ or R^{6a} is selected from C₁₋₆alkoxy, N-C₁₋₆alkylamino and N,N-diC₁₋₆alkylamino, and/or
(iv) R⁵ is a group of formula III-k, III-l, III-o or III-p, and/or
(vi) both of R²¹ and R²² within a —(CR²¹R²²)ₛ₁— or —(CR²¹R²²)ₛ₂— are C₁₋₄alkyl; or
(vii) at least one of R²¹ or R²² within a —(CR²¹R²²)ₛ₁— or —(CR²¹R²²)ₛ₂— is a C₁₋₄alkyl which is substituted by a group ZR³⁰.
   More preferably, the compound of formula (I) includes a least one of the following groups:
(i) R³ is a group of formula (IIc) or (IId) wherein J is a group of the formula:
   —(CH₂)ₛ—C(O)—(CH₂)ₛ-L-(CH₂)ₛ—, and/or
(iii) at least one group R⁶ or R^{6a} is selected from C₁₋₆alkoxy, N-C₁₋₆alkylamino and N,N-diC₁₋₆alkylamino, and/or
(iv) R⁵ is a group of formula III-k, III-l, III-o or III-p, and/or
(vi) both of R²¹ and R²² within a —(CR²¹R²²)ₛ₁— or —(CR²¹R²²)ₛ₂— are C₁₋₄alkyl; or
(vii) at least one of R²¹ or R²² within a —(CR²¹R²²)ₛ₁— or —(CR²¹R²²)ₛ₂— is a C₁₋₄alkyl which is substituted by a group ZR³⁰,

In particular, the compound of formula (I) is as defined above, with the proviso that the compound must contain at least one of the following groups:
(i) J is a group of the formula: —(CH₂)ₛ—C(O)—(CH₂)ₛ-L-(CH₂)ₛ—, and/or
(ii) R² is selected from hydrogen or optionally substituted C₁₋₆alkyl, and/or
(iii) R⁶ and R^{6a} are independently selected from C₁₋₆alkoxy, N-C₁₋₆alkylamino and N,N-diC₁₋₆alkylamino, and/or
(iv) R⁵ is a group of formula III-o or III-p, and/or
(v) R⁸ is substituted by R¹³, and/or
(vi) a CH₂ group within —(CH₂)ₛ₁— or —(CH₂)ₛ₂— is di-substituted with C₁₋₄alkyl;
   or a salt, solvate or pro-drug thereof.

According to a further feature of the first aspect of the invention there is provided a compound of Formula (I) as defined above:

with the proviso that the compound must contain at least one of the following groups:

(i) J is a group of the formula: —(CH₂)ₛ—C(O)—(CH₂)ₛ-L-(CH₂)ₛ—, and/or
(ii) R² is selected from hydrogen or optionally substituted C₁₋₆alkyl, and/or
(iii) R⁶ an R^{6a} are independently selected from C₁₋₆alkoxy, N-C₁₋₆alkylamino and N,N-diC₁₋₆alkylamino, and/or
(iv) R⁵ is a group of formula III-o or III-p, and/or
(v) R⁸ is substituted by R¹³;
   or a salt, solvate or pro-drug thereof.

According to a further feature of the first aspect of the invention there is provided a pharmaceutical formulation comprising a compound of Formula (I), or salt, pro-drug or solvate thereof, and a pharmaceutically acceptable diluent or carrier.

According to a further feature of the first aspect of the invention there is provided the following uses of a compound of Formula (I), or salt, pro-drug or solvate thereof:
(a) the use in the manufacture of a medicament for antagonising gonadotropin releasing hormone activity;
(b) the use in the manufacture of a medicament for administration to a patient, for reducing the secretion of luteinizing hormone by the pituitary gland of the patient; and
(c) the use in the manufacture of a medicament for administration to a patient, for therapeutically treating and/or preventing a sex hormone related condition in the patient, preferably a sex hormone related condition selected from prostate cancer and pre-menopausal breast cancer.

According to a further aspect of the invention there is provided a method of antagonising gonadotropin releasing hormone activity in a patient, comprising administering a compound of Formula (I), or salt, pro-drug or solvate thereof, to a patient.

Whilst pharmaceutically-acceptable salts of compounds of the invention are preferred, other non-pharmaceutically-acceptable salts of compounds of the invention may also be useful, for example in the preparation of pharmaceutically-acceptable salts of compounds of the invention.

Whilst the invention comprises compounds of the invention, and salts, pro-drugs or solvates thereof, in a further embodiment of the invention, the invention comprises compounds of the invention and salts thereof.

In the present specification, unless otherwise indicated, an alkyl, alkylene, alkenyl or alkynyl moiety may be linear or branched. The term “alkylene” refers to the group —CH₂—. Thus, C₈ alkylene for example is —(CH₂)₈—. For avoidance of doubt the term C₀alkyl within the group C₀₋₅alkyl is a direct bond.

The term ‘propylene’ refers to trimethylene and the branched alkyl chains —CH(CH₃)CH₂— and —CH₂—CH(CH₃)—. The straight chain propylene di-radical is preferred, i.e. —CH₂CH₂CH₂—. Specific propylene radicals refer to the particular structure, thus the term, propyl-2-ene refers to the group —CH₂—CH(CH₃)—. Similar notation is used for other divalent alkyl chains such as butylene.

The term ’2-propenyl’ refers to the group —CH₂—CH═CH—.

The term “aryl” refers to phenyl or naphthyl.

The term “carbamoyl” refers to the group —C(O)NH₂.

The term ”halo” refers to fluoro, chloro, bromo or iodo.

The term “carbocyclyl” or “carbocyclic ring” includes rings of carbon atoms, for example of from 3-12 carbon atoms, which may be saturated, unsaturated (such as aryl or aromatic rings such as phenyl or napthyl) or partially unsaturated. They may be mono- or bi-cyclic.

The term “heterocyclyl” or ”heterocyclic ring” refers to a 4-12 membered, preferably 5-10 membered aromatic mono or bicyclic ring or a 4-12 membered, preferably 5-10 membered saturated or partially saturated mono or bicyclic ring, said aromatic, saturated or partially unsaturated rings containing up to 5 heteroatoms independently selected from nitrogen, oxygen or sulphur, linked via ring carbon atoms or ring nitrogen atoms where a bond from a nitrogen is allowed, for example no bond is possible to the nitrogen of a pyridine ring, but a bond is possible through the 1-nitrogen of a pyrazole ring. Examples of 5- or 6-membered aromatic heterocyclic rings include pyrrolyl, furanyl, imidazolyl, triazolyl, pyrazinyl, pyrimidinyl, pyridazinyl, pyridinyl, isoxazolyl, oxazolyl, 1,2,4 oxadiazolyl, isothiazolyl, thiazolyl and thienyl. A 9 or 10 membered bicyclic aromatic heterocyclic ring is an aromatic bicyclic ring system comprising a 6-membered ring fused to either a 5 membered ring or another 6 membered ring. Examples of 5/6 and 6/6 bicyclic ring systems include benzofuranyl, benzimidazolyl, benzthiophenyl, benzthiazolyl, benzisothiazolyl, benzoxazolyl, benzisoxazolyl, indolyl, pyridoimidazolyl, pyrimidoimidazolyl, quinolinyl, isoquinolinyl, quinoxalinyl, quinazolinyl, phthalazinyl, cinnolinyl and naphthyridinyl. Examples of saturated or partially saturated heterocyclic rings include pyrrolinyl, pyrrolidinyl, morpholinyl, piperidinyl, piperazinyl, dihydropyridinyl, benzodioxyl and dihydropyrimidinyl. This definition further comprises sulphur-containing rings wherein the sulphur atom has been oxidised to an S(O) or S(O2) group.

The term ”heteroaryl” refers to a 5-6 membered aromatic ring or 5-6 membered unsaturated ring containing from 1 to 4 heteroatoms independently selected from O, N and S.

The term “aromatic ring” refers to a 5-10 membered aromatic mono or bicyclic ring optionally containing up to 5 heteroatoms independently selected from nitrogen, oxygen or sulphur. Examples of such ”aromatic rings” include: phenyl, pyrrolyl, pyrazolyl, furanyl, imidazolyl, triazolyl, pyrazinyl, pyrimidinyl, pyridazinyl, pyridinyl, isoxazolyl, oxazolyl, 1,2,4 oxadiazolyl, isothiazolyl, thiazolyl and thienyl. Preferred aromatic rings include phenyl, thienyl and pyridyl.

The term “amino acid derivative” is defined as that derived from the coupling of an L- or D-amino acid with a carboxyl group via an amide bond. This bond is formed via the amino group on the amino acid backbone. Amino acid derivatives include those derived from natural and non-natural amino acids, preferably natural amino acids and include α-amino acids β-amino acids and γ-amino acids. For the avoidance of doubt amino acids include those with the generic structure: 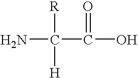
where R is the amino acid side chain. The definition of amino acid also includes amino acid analogues which have additional methylene groups within the amino acid backbone, for example β-alanine and amino acids which are not naturally occurring such as cyclohexylalanine.

Preferred amino acids include glycine, alanine, valine, leucine, isoleucine, methionine, proline, phenylalanine, tryptophan, serine, threonine, cysteine, tyrosine, asparaginine, glutamine, aspartic acid, glutamic acid, lysine, histidine, β-alanine and ornithine. More preferred amino acids include glutamic acid, serine, threonine, glycine, alanine, β-alanine and lysine. Yet more preferred amino acids include: alanine, asparagine, glycine, leucine, methionine, serine and threonine and non-natural amino acids with the following side chains: CH₃—S—CH₂—, CH₃—CH₂—, CH₃—CH(OH)— and HO—CH₂CH₂—.

Especially preferred amino acids include alanine, leucine, methionine and serine and non-natural amino acids with the following side chains: CH₃—S—CH₂—, CH₃—CH₂—, CH₃—CH(OH)— and HO—CH₂CH₂—.

An amide of an amino acid is defined as amino acid as defined above wherein the carboxy group on the amino acid backbone has been converted to an amide, or where present the carboxyl group on an amino acid side chain has been converted to an amide. Optionally the amino group of the amide group is substituted by C₁₋₄alkyl.

For example, the equivalent generic structure to the generic amino structure above is:

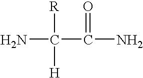

The symbol 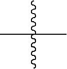
denotes where the respective group is linked to the remainder of the molecule.

For the avoidance of doubt where any two groups or integers appear more than once within the same definition, for example, —(CH₂)ₛ-L-(CH₂)ₛ—, then these can be the same or different.

For the avoidance of doubt, where several groups together form a ring, for example: ‘the group 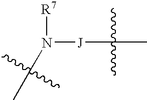
together forms an optionally substituted heterocyclic ring containing 4-7 carbon atoms’, then the groups shown cyclises to form a ring, i.e 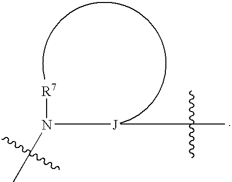
For example in Example 5 hereinafter, this group forms a piperidine ring.

The term C₁₋₃perfluoroalkyl refers to a C₁₋₃alkyl chain in which all hydrogens have been replaced with a fluorine atom. Examples of C₁₋₃perfluoroalkyl include trifluoromethyl, pentafluoroethyl and 1-trifluoromethyl-1,2,2,2-tetrafluoroethyl. Preferably C₁₋₃perfluoroalkyl is trifluromethyl.

Examples of C₁₋₈alkyl include: methyl, ethyl, propyl, isopropyl, butyl, iso-butyl, tert-butyl and 2-methyl-pentyl; examples of C₁₋₈alkylene include: methylene, ethylene and 2-methyl-propylene; examples of C₁₋₆alkenyl include allyl (2-propenyl) and 2-butenyl, examples of C₁₋₆alkynyl include 2-propynyl and 3-butynyl, examples of haloC₁₋₆alkyl include fluoroethyl, chloropropyl and bromobutyl, examples of hydroxyC₁₋₆alkyl include hydroxymethyl, hydroxyethyl and hydroxybutyl, examples of C₁₋₈alkoxy include methoxy, ethoxy and butyloxy; examples of C₁₋₄alkoxyC₁₋₄alkyl include methoxyethyl, propoxybutyl and propoxymethyl, examples of C₁₋₆alkanoyl include formyl, ethanoyl, propanoyl or pentanoyl, examples of N-C₁₋₄alkylamino include N-methylamino and N-ethylamino; examples of N,N-di-C₁₋₄alkylamino include N,N-dimethylaminoethyl, N,N-dimethylaminopropyl and N,N-dipropylaminoethyl, examples of HO—C₂₋₄alkyl-NH include hydroxymethylamino hydroxyethylamino and hydroxypropylamino, examples of HO—C₂₋₄alkyl-N(C₁₋₄alkyl) include N-methyl-hydroxymethylamino, N-ethyl-hydroxyethylamino, and N-propyl-hydroxypropyamino, examples of C₁₋₆alkyl-S(Oₙ)— include methylthio, methylsulphinyl, ethylsulphinyl, ethylsulphonyl and propylsulphonyl, examples of arylC₁₋₆alkyl include benzyl, phenethyl and phenylbutyl, examples of heterocyclylC₁₋₆alkyl include pyrrolidin-1-yl ethyl, imidazolylethyl, pyridylmethyl and pyrimidinylethyl.

It is to be understood that, insofar as certain of the compounds of the invention may exist in optically active or racemic forms by virtue of one or more asymmetric carbon atoms, the invention includes in its definition any such optically active or racemic form which possesses the property of antagonizing gonadotropin releasing hormone (GnRH) activity. The synthesis of optically active forms may be carried out by standard techniques of organic chemistry well known in the art, for example by synthesis from optically active starting materials or by resolution of a racemic form. Similarly, activity of these compounds may be evaluated using the standard laboratory techniques referred to hereinafter.

The invention also relates to any and all tautomeric forms of the compounds of the different features of the invention that possess the property of antagonizing gonadotropin releasing hormone (GnRH) activity.

It will also be understood that certain compounds of the present invention may exist in solvated, for example hydrated, as well as unsolvated forms. It is to be understood that the present invention encompasses all such solvated forms which possess the property of antagonizing gonadotropin releasing hormone (GnRH) activity.

Preferred compounds of Formula (I) are those wherein any one of the following or any combination of the following apply.

Preferably R¹ is selected from hydrogen, optionally substituted C₁₋₆alkyl or optionally substituted arylC₁₋₆alkyl, wherein the optional substitutuents are as described herein. More preferably R¹ represents hydrogen, unsubstituted C₁₋₆alkyl or optionally substituted arylC₁₋₆alkyl. Yet more preferably R¹ represents hydrogen, methyl, ethyl, tert-butyl or benzyl: Most preferably R¹ represents hydrogen.

Preferably optional substituents on R¹ are independently selected from: fluoro and C₁₋₄alkoxy. Most preferably R¹ is unsubstituted.

Most preferably R¹ is unsubstituted.

Preferably R² is an optionally substituted monocyclic aromatic ring structure, wherein the optional substitutuents are as described herein. Most preferably R² represents optionally substituted phenyl, wherein the optional substitutuents are as described herein.

In another embodiment of the invention R² is hydrogen or optionally substituted C₁₋₆alkyl wherein the optional substituents are as described herein.

In another embodiment of the invention R² is hydrogen or optionally substituted C₁₋₆alkyl wherein the optional substituents are as described herein and R¹ is optionally substituted arylC₁₋₆alkyl, wherein the optional substitutuents are as described herein.

Preferably optional substituents on R² are independently selected from methyl, ethyl, methoxy, ethoxy, tert-butoxy, F or Cl. Most preferably optional substituents on R² are independently selected from methyl, F or Cl. Preferably R² bears 1, 2 or 3 substituents, most preferably 2 substituents.

Most preferably R² represents

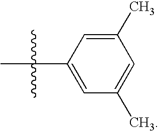

Preferably R³ is selected from a group of Formula (IIc) and Formula (IId). Most preferably R³ is a group of Formula (IId).

Preferably R⁴ is selected from hydrogen, methyl, ethyl, chloro or bromo. Further preferably R⁴ is selected from hydrogen or chloro. Most R⁴ is hydrogen.

Preferably R⁵ is selected from a group of Formula III-a , III-g, III-h, III-i, III-j, III-k, or III-l: 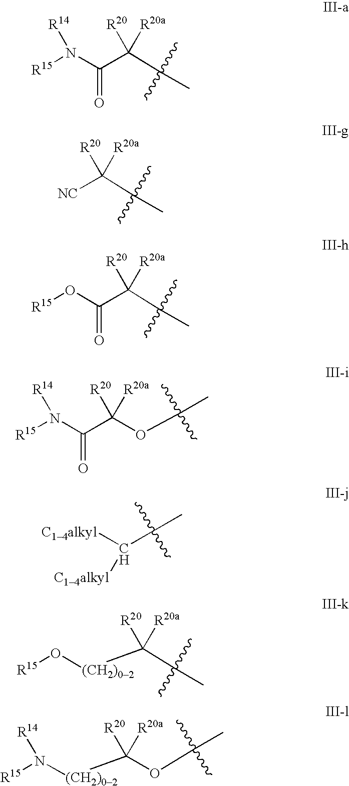
wherein R²⁰, R^{20a}, R¹⁴ and R¹⁵ are as defined above.

More preferably the group of Formula (III) is selected from one of the following groups: 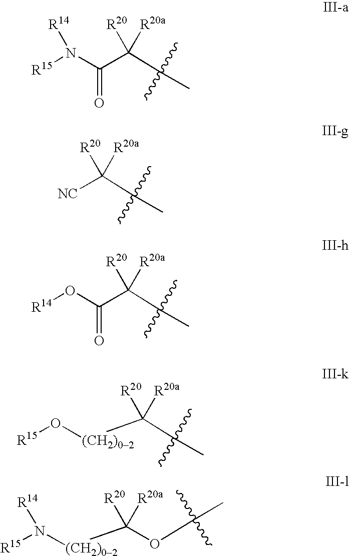
wherein R²⁰, R^{20a} R¹⁴ and R¹⁵ are as defined above.

Further preferably the group of Formula (III) is selected from one of the following groups: 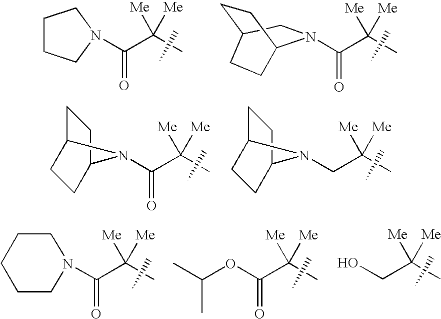
wherein Me represents methyl and het is as defined above.

Yet further preferably the group of Formula (III) is selected from one of the following groups:

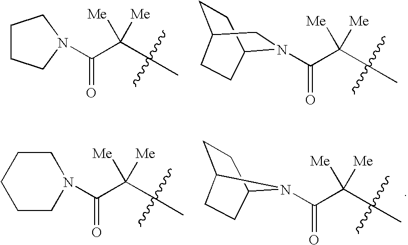

Most preferably the group of Formula (III) is:

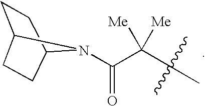

Preferably R⁶ and R^{6a} are independently selected from hydrogen, fluoro, optionally substituted C₁₋₆alkyl (wherein any optional substitutents are selected from R¹²) or R⁶ and R^{6a} taken together and the carbon atom to which they are attached form a carbocyclic ring of 3-7 atoms. For instance, R⁶ and R^{6a} are independently selected from hydrogen, fluoro, C₁₋₆alkyl, C₁₋₆alkoxy, or R⁶ and R^{6a} taken together and the carbon atom to which they are attached form a carbocyclic ring of 3-7 atoms. More preferably R⁶ and R^{6a} are independently selected from hydrogen, unsubstituted C₁₋₆alkyl or R⁶ and R^{6a} taken together and the carbon atom to which they are attached form a carbocyclic ring of 3-7 atoms. Yet more preferably R⁶ and R^{6a} are independently selected from hydrogen, methyl or R⁶ and R^{6a} taken together and the carbon atom to which they are attached form cyclopropyl. Further preferably R⁶ is hydrogen and R^{6a} is methyl. Most preferably R⁶ and R^{6a} are both hydrogen.

In a particular embodiment, at least one of R⁶ or R^{6a} is selected from C₁₋₆alkoxy, N-C₁₋₆alkylamino and N,N-diC₁₋₆alkylamino, suitably C₁₋₆alkoxy such as methoxy. The other of R⁶ or R^{6a} is preferably hydrogen.

Preferably R⁷ is selected from: hydrogen or C₁₋₄alkyl. More preferably R⁷ is hydrogen or methyl. Most preferably R⁷ is hydrogen.

Preferably R⁸ is selected from
(i) hydrogen, C₁₋₆alkyl, C₂₋₆alkenyl, haloC₁₋₆alkyl, hydroxy, cyano, C₁₋₆alkylS(Oₙ)—, —O—R^{b}, C₁₋₄alkoxyC₁₋₄alkyl, —C(O)—R^{b}, C(O)O—R^{b}, —NH—C(O)—R^{b},
   N,N-di-C₁₋₄alkylamino, —S(Oₙ)NR^{b}R^{c}
   where R^{b} and R^{c} are as defined above and are preferably independently selected from hydrogen and C₁₋₄alkyl, and n is 0, 1 or 2;
(ii) C₄₋₇heterocyclyl, optionally substituted by up to 3 groups selected from R¹² and R¹³, or
(iii) phenyl or C₃₋₇carbocyclyl; each of which is optionally substituted by up to 3 groups selected from R¹² and R¹³.

Particular examples of C₄₋₇heterocyclyl groups R⁸ include azirinyl, azetidinyl, pyrrolidinyl, pyrazolinyl, pyrazolidinyl, imidazolinyl, imidazolidinyl, piperidinyl, piperazinyl, hexahydropyrimidinyl, hexahydropyridazinyl, hexahydrotriazinyl, tetraydrotriazinyl, dihydrotriazinyl, tetrahydrofuranyl, dioxolanyl, tetrahydropyranyl, dioxanyl, trioxanyl, tetrahydrothienyl, 1-oxotetrahydrothienyl, 1,1-dioxotetrahydrothienyl tetrahydrothiopyran, 1-oxotetrahydrothiopyran, 1,1-dioxotetrahydrothiopyran, dithianyl, trithianyl, morpholinyl, oxathiolanyl, oxathianyl, thiomorpholinyl, thiazinanyl, 1-oxo-thiomorpholinyl, 1,1-dioxo-thiomorpholinyl, 1,1-dioxo-isothiazolidiyl, thiazolidinyl, pyrrolyl, imidazolyl, triazolyl, pyridyl, pyrimidinyl, pyrazinyl, pyridazinyl, triazinyl, thiazolyl, thiadiazolyl, thiadiazinyl, oxazolyl, isoxazolyl, oxadiazolyl, furazanyl, octahydropyrrolopyrrolyl, octahydropyrrolopyrrolyl, benzotriazolyl, dihydrobenzotriazolyl, indolyl, indolinyl, benzimidazolyl, 2,3-dihydrobenzimidazoly, benzotriazolyl 2,3-dihydro benzotriazolyl quinolinyl, isoquinolinyl, cinnolinyl, phthalazinyl, quinazolinyl, quinozalinyl, naphthyridinyl, pteridinyl, benzodioxolyl, tetrahydrodioxolopyrrolyl, 1,5-dioxa-9-azaspiro[5.5]undecanyl or 8-oxa-3-azabicyclooctanyl; each of which is optionally substituted by up to 3 groups selected from R¹² and R¹³.

Further preferably R⁸ is selected from
(i) hydrogen, C₁₋₆alkyl, C₂₋₆alkenyl, haloC₁₋₆alkyl, hydroxy, cyano, C₁₋₆alkylS(Oₙ)—, —O—R^{b}, C₁₋₄alkoxyC₁₋₄alkyl, —C(O)—R^{b}, C(O)O—R^{b}, —NH—C(O)—R^{b}, N,N-di-C₁₋₄alkylamino, —S(Oₙ)NR^{b}R^{c}
   where R^{b} and R^{c} are independently selected from hydrogen and C₁₋₆alkyl, and n is 0, 1 or2;
   preferably selected from: hydrogen, methyl, isopropyl, t-butyl, 1-methylethyl, allyl, fluoroethyl, hydroxy, cyano, ethylsulphonyl, methoxy, 1-methyl-2-methoxyethyl, acetyl, t-butoxycarbonyl, acetylamino, dimethylamino, diethylamino, (1-methylethyl)amino, isopropylamino or aminosulphonyl;
(ii) azetidinyl, furanyl, tetrahydrofuranyl, tetrahydropyranyl, pyrrolidinyl, piperidinyl, piperazinyl, hexahydropyrimidinyl, morpholinyl, tetrahydrothienyl, 1,1-dioxotetrahydrothienyl, thiomorpholinyl, 1-oxo-thiomorpholinyl, 1,1-dioxo-thiomorpholinyl, imidazolyl, triazolyl, thienyl, thiazolyl, isoxazolyl, pyridyl, pyrimidinyl, pyrazinyl, tetrahydro-3aH-[1,3]dioxolo[4,5-c]pyrrolyl, 1,5-dioxa-9-azaspiro[5.5]undecanyl, 8-oxa-3-azabicyclo[3.2.1]octanyl, benzodioxolyl, 2,3-dihydrobenzotriazolyl, 1,2-dihydroquinolinyl or octahydropyrrolo[3,4-c]pyrrolyl; each of which is optionally substituted by up to 3 groups selected from R¹² and R¹³; or
(iii) phenyl or C₃₋₇carbocyclyl, such as cyclohexyl, each of which is optionally substituted by up to 3 groups selected from R¹² and R¹³.

Yet further preferably R⁸ is selected from
(i) phenyl optionally substituted by up to 3 groups selected from R¹² and R¹³ or naphthyl;
(ii) furanyl, tetrahydropyranyl, pyrrolidinyl, piperazinyl, morpholinyl, 1,1-dioxo-thiomorpholinyl, thienyl, triazolyl, pyridyl, pyrimidinyl, pyrazinyl, piperidinyl, tetrahydro-3aH-[1,3]dioxolo[4,5-c]pyrrolyl, benzodioxolyl, 1,2-dihydroquinolinyl or 2,3-dihydrobenzotriazolyl; each of which is optionally substituted by up to 3 groups selected from R¹² and R¹³;or
(iii) C₃₋₇carbocyclyl (preferably cyclohexyl or cylopentyl, more preferably cyclohexyl) optionally substituted by up to 3 groups selected from R¹² and R¹³;

Yet further preferably R⁸ is selected from optionally substituted C₄₋₇heterocyclyl selected from: morpholinyl, piperidinyl, pyrrolidinyl, azetidinyl, imidazolyl and thiazolyl, wherein the optional substitutents are selected from R¹² and R¹³

Most preferably R⁸ is optionally substituted C₄₋₇heterocyclyl selected from: morpholinyl, piperidinyl or pyrrolidinyl, wherein the optional substitutents are selected from R¹² and R¹³.

In an alternative embodiment, R⁸ is an optionally substituted C₅₋₇ heterocyclyl selected from pyrrolyl, furanyl pyridyl, pyrrolidinyl, morpholinyl, thienyl, thiazolyl or benzodioxolyl; each of which is optionally substituted by up to 3 groups selected from R¹² and R¹³.

More preferably optional substituents on R⁸ are selected from: cyano, hydroxy, oxo, nitro, halo, trifluromethyl, C₁₋₄alkyl, hydroxyC₁₋₄alkyl, C₁₋₄alkoxy, C₁₋₄alkoxyC₁₋₄allyl, C₁₋₄alkanoyl, R⁹OC(O)(CH₂)_{w}—, R⁹R¹⁰N(CH₂)_{w}—, R⁹R¹⁰ONC(O)(CH₂)_{w}—, R⁹R¹⁰NC(O)(CH₂)_{w}—, R⁹R¹⁰NC(O)N(R⁹)(CH₂)_{w}—, R⁹OC(O)N(R⁹)(CH₂)_{w}—, or halo, wherein w is an integer between 0 and 4 and R⁹ and R¹⁰ are selected from: hydrogen, C₁₋₄alkyl, C₁₋₄alkylsulphonyl and C₃₋₇carbocyclyl.

Further preferably optional substituents on R⁸ are selected from: cyano, hydroxy, oxo, amino, N,N-diC₁₋₄alkyamino, N,N-diC₁₋₄alkyaminoC₁₋₄alkyl, N′-C₁₋₄alkylureido, N-C₁₋₄alkylsulphonylamino, N,N-di-C₁₋₄alkylsulphonylamino, nitro, halo, trifluoromethyl, C₁₋₄alkyl, hydroxyC₁₋₄alkyl, C₁₋₄alkoxy, C₁₋₄alkoxyC₁₋₄alkyl, C₁₋₄alkanoyl, C₁₋₄alkoxycarbonylamino and C₃₋₇carbocyclylcarbonylamino.

More preferably optional substituents on R⁸ are selected from: cyano, hydroxy, hydroxyC₁₋₄alkyl, oxo, methyl, ethyl, t-butyl, methoxy, acetyl, amino, N,N-dimethylamino, N′-isopropylureido, N′-cyclohexylureido, N-methylsulphonylamino, N,N-dimethylsulphonylamino, nitro, chloro, fluoro, trifluoromethyl, isopropoxycarbonylamino and cyclopentylcarbonylamino.

Further preferably optional substituents on R⁸ are selected from: hydroxy, methyl, ethyl, hydroxymethyl, hydroxyethyl, methoxy, ethoxy, methoxymethyl, ethoxymethyl and methoxyethyl. Most preferably optional substituents on R⁸ are selected from: hydroxy.

In a further embodiment of the invention optional substituents on R⁸ are selected from: C₁₋₄alkoxy, fluoro, C₁₋₄alkylsulphonylamino, C₁₋₄alkanoylamino, C₁₋₄alkylureido and C₁₋₄alkoxycarbonylamino.

Preferably R²⁰ and R^{20a} are independently selected from hydrogen and C₁₋₄alkyl. More preferably R²⁰ and R^{20a} are independently selected from hydrogen, methyl and ethyl. Most preferably R²⁰ and R^{20a} are both methyl.

Preferably R¹⁴ is hydrogen or methyl. Most preferably R¹⁴ is hydrogen.

Suitably A is selected from:
(i) a direct bond;
(ii) optionally substituted C₁₋₅alkylene wherein the optional substituents are independently selected from: hydroxy, hydroxyC₁₋₆alkyl, C₁₋₆alkyl, C₁₋₆alkoxy, C₁₋₄alkoxyC₁₋₄alkyl, aryl or arylC₁₋₆alkyl;
(iii) a carbocyclic ring of 3-7 atoms;
(iv) a carbonyl group or —C(O)—C(R^{d}R^{d})—, wherein each R^{d} is independently selected from hydrogen and C₁₋₂alkyl.

Preferably A is selected from a direct bond, optionally substituted C₁₋₅alkylene wherein the optional substituents are independently selected from: hydroxy, hydroxyC₁₋₆alkyl, C₁₋₆alkyl, C₁₋₆alkoxy, C₁₋₄alkoxyC₁₋₄alkyl, aryl or arylC₁₋₆alkyl; or A is carbonyl or —C(O)—C(R^{d}R^{d})—, wherein R^{d} is independently selected from hydrogen and C₁₋₂alkyl.

Further preferably A is selected from C₁₋₅alkylene optionally substituted with C₁₋₄alkyl or C₁₋₄alkoxy, carbonyl or carbonylmethyl. Yet further preferably A is a direct bond or ethylene. Most preferably A is methylene.

Suitably B is selected from:
(i) a direct bond;
(ii) a group of Formula (IV) 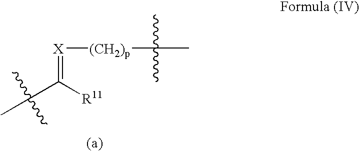
wherein:
   X is selected from N or CH,
   wherein at position (a) Formula (IV) is attached to the nitrogen atom and the (CH₂)ₚ group is attached to R⁸; and
(iii) a group independently selected from: optionally substituted C₁₋₆alkylene, optionally substituted C₃₋₇cycloalkyl, optionally substituted C₃₋₆alkenylene, optionally substituted C₃₋₆alkynyl, (C₁₋₅alkyl)ₐₐ-S(Oₙ)—(C₁₋₅alkyl)_{bb}-, —(C₁₋₅alkyl)ₐₐ-O—(C₁₋₅alkyl)_{bb}-, —(C₁₋₅alkyl)ₐₐ-C(O)—(C₁₋₅alkyl)_{bb}- or —(C₁₋₅alkyl)ₐₐ-N(R^{14a})—(C₁₋₅alkyl)_{bb},
   wherein R^{14a} is a group R¹⁴ as defined above, or R^{14a} and the (C₁₋₅alkyl)ₐₐ or (C₁₋₅alkyl)_{bb} chain can be joined to form a heterocyclic ring, wherein the combined length of (C₁₋₅alkyl)ₐₐ and (C₁₋₅alkyl)_{bb} is less than or equal to C₅alkyl and wherein the optional substituents are independently selected from R¹².

Particular examples of R¹¹ include hydrogen, C₁₋₄alkyl or N(R²³R²⁴), where R²³ and R²⁴ are independently selected from hydrogen or C₁₄alkyl.

Preferably B is selected from optionally substituted C₁₋₆alkylene, optionally substituted C₃₋₆alkenylene, —(C₁₋₅alkyl)ₐₐ-O—(C₁₋₅alkyl)_{bb}, —(C₁₋₅alkyl)ₐₐC(O)—(C₁₋₅alkyl)_{bb}-, —(CH₂)ₛ₁—C(O)N(R^{14a})—(CH₂)ₛ₂—, or the group 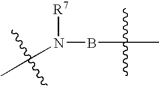
forms an optionally substituted C₄₋₇heterocyclic ring, wherein R^{14a} is as defined above, and is suitably selected from hydrogen or C₁₋₄alkyl (preferably hydrogen) and aa and bb are independently 0 to 1.

More preferably B is C₁₋₆alkylene, C₃₋₆alkenylene, —(C₁₋₅alkyl)ₐₐ-O—(C₁₋₅alkyl)_{bb}-, —(C₁₋₅alkyl)ₐₐ-C(O)—(C₁₋₅alkyl)_{bb}-, —(CH₂)ₐₐ—C(O)N(R^{14a})(C₁₋₅alkyl)_{bb}, or the group 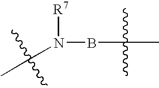
forms an optionally substituted saturated C₄₋₇heterocyclic ring, wherein R^{14a} is as defined above, aa and bb are independently 0 or 1 and wherein C₁₋₆alkylene is optionally substituted by hydroxy.

Further preferably B is unsubstituted C₁₋₆alkylene, C₃₋₆alkenylene —(C₁₋₅alkyl)ₐₐ-O—(C₁₋₅alkyl)_{bb}-, —(C₁₋₅alkyl)ₐₐ-C(O)— or the group 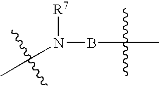
forms an optionally substituted saturated C₄₋₇heterocyclic ring selected from: azetidinyl, pyrrolidinyl, pyrazolinyl, pyrazolidinyl, imidazolinyl, imidazolidinyl, piperidinyl, piperazinyl, hexahydropyrimidinyl, hexahydropyridazinyl, hexahydrotriazinyl, tetraydrotriazinyl, dihydrotriazinyl, morpholinyl, thiomorpholinyl, thiazinanyl, thiazolidinyl, 1,5-dioxa-9-azaspiro[5.5]undecanyl or octahydropyrrolopyrrolyl, wherein the optional substituents are selected from cyano, halo, hydroxy, oxo, C₁₋₄alkyl, C₁₋₄alkoxy, C₁₋₄alkanoyl, carboxyl, C₁₋₆alkyl, C₁₋₆alkoxycarbonylC₀₋₄alkyl, aminocarbonylC₀₋₄alkyl, N-C₁₋₆alkyaminocarbonylC₀₋₄alkyl or N,N-C₁₋₆alkyaminocarbonylC₀₋₄alkyl.

Further preferably the optional substituents are selected from: cyano, hydroxy, oxo, C₁₋₄alkyl, C₁₋₄alkoxy and C₁₋₄alkanoyl, and aa and bb are independently 0 or 1. In particular B is C₁₋₆alkylene which is optionally substituted by hydroxy.

Yet further preferably B is selected from: methylene, ethylene, propylene, propyl-2-ene, butylene, pentylene, 2-propenyl, propoxy, ethoxyethyl, methylcarbonyl or methylcarbonylamino. or the group 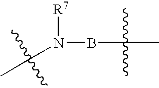
forms a C₄₋₇heterocyclic ring selected from: pyrrolidinyl, piperidinyl, or piperazinyl, wherein the optional substituents are selected from oxo.

Most preferably B is selected from ethylene or butylene.

In another embodiment of the invention preferably B is selected from optionally substituted C₁₋₆alkylene or the group 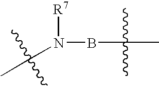
forms a C₅₋₇heterocyclic ring. Preferably B is selected from unsubstituted C₁₋₆alkylene or the group 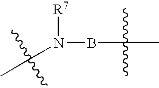
forms a saturated C₅₋₇heterocyclic ring. Most preferably B is selected from methylene, ethylene, propylene, butylene or or the group 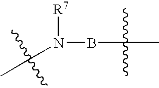
forms a saturated C₅₋₇heterocyclic ring selected from piperidinyl or piperazinyl.

When R³ is selected from a group of Formula (IIc) or Formula (IId) then the group 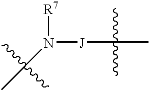
preferably forms an optionally substituted heterocyclic ring containing 4-7 carbons atoms, wherein the optional substituents are selected from 1 or 2 substituents independently selected from R¹² and R¹³.

Further preferably the group 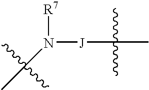
forms an optionally substituted saturated C₄₋₇heterocyclic ring selected from: azetidinyl, pyrrolidinyl, pyrazolinyl, pyrazolidinyl, imidazolinyl, imidazolidinyl, piperidinyl, piperazinyl, hexahydropyrimidinyl, hexahydropyridazinyl, hexahydrotriazinyl, tetraydrotriazinyl, dihydrotriazinyl, morpholinyl, thiomorpholinyl, thiazinanyl, thiazolidinyl or octahydropyrrolopyrrolyl, wherein the optional substituents are selected from oxo, hydroxy, C₁₋₄alkyl, hydroxyC₁₋₄alkyl, C₁₋₄alkoxy and C₁₋₄alkoxyC₁₋₄alkyl. Further preferably the optional substitutents are selected from: oxo, hydroxy, methyl, ethyl, hydroxymethyl, hydroxyethyl, methoxymethyl.

Further preferably the group 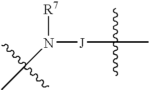
forms an optionally substituted saturated C₄₋₇heteocyclic ring selected from: pyrrolidinyl, piperidinyl or piperazinyl, wherein the optional substituents are selected from C₁₋₄alkoxy.

Most preferably the group 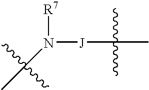
forms an optionally substituted saturated C₄₋₇heteocyclic ring selected from: piperazinyl or piperidinyl.

In a particular embodiment, J is a group of the formula: —(CH₂)ₛ-L-(CH₂)ₛ— or —(CH₂)ₛ—C(O)—(CH₂)ₛ-L-(CH₂)ₛ— wherein when s is greater than 0, the alkylene group is optionally substituted by 1 to 2 group selected from R¹². In particular, the group J is a group of formula —(CH₂)ₛ—C(O)—(CH₂)ₛ-L-(CH₂)ₛ—.

In this instance, at least one and suitably all s groups are 0.

Groups L are optionally substituted aryl or optionally substituted heterocyclyl groups. Suitable optional substituents for groups L include those listed above for R¹². Preferably L is unsubstituted other than by the adjacent —(CH₂)ₛ— groups.

In particular, L is an optionally substituted heterocyclic group as defined above. In particular it is a 4-12 membered, preferably 5-10 membered saturated or partially saturated mono or bicyclic ring includes at least one nitrogen atom. Preferably the nitrogen atom is linked to an adjacent —(CH₂)ₛ group. Examples of saturated or partially saturated heterocyclic rings include azetindinyl, pyrrolinyl, pyrrolidinyl, morpholinyl, piperidinyl, piperazinyl, dihydropyridinyl, benzodioxyl and dihydropyrimidinyl. A particularly preferred group L is azetindinyl.

Within the group K, each R²¹ and R²² is independently selected from hydrogen, hydroxy or C₁₋₄alkyl, which is optionally substituted by a group ZR³⁰ where Z is oxygen or a group S(O)ₙ where n is as described above, and R³⁰ is hydrogen or C₁₋₄alkyl. Particular examples of R³⁰ are hydrogen or methyl. Preferably in this case, the integer n is 0. Suitable examples of the group ZR³⁰ are hydroxy and thiomethyl. In a particular embodiment of the invention, at least one group R²¹ or R²² is C₁₋₄alkyl substituted by a group ZR³⁰.

Where one of R²¹ or R²² is C₁₋₄alkyl substituted by a group ZR³⁰, the other is suitably hydrogen.

In an alternative embodiment, both R²¹ and R²² are C₁₋₄alkyl such as methyl.

Preferably K is selected from: a direct bond, —(CH₂)ₛ—, —(CH₂)ₛ—O—(CH₂)ₛ—, —(CH₂)ₛ—C(O)—(CH₂)ₛ—, —(CH₂)ₛ—N(R¹⁴)—(CH₂)ₛ—, —(CH₂)ₛ—C(O)N(R¹⁴)—(CH₂)ₛ—, —(CH₂)ₛ—N(R¹⁴)C(O)—(CH₂)ₛ—, —(CH₂)ₛ—S(O)₂N(R¹⁴)—(CH₂)ₛ—, or —(CH₂)ₛ—NHS(O)₂—(CH₂)ₛ—, wherein s is independently selected from 0, 1, 2, 3 or 4, R¹⁴ is selected from hydrogen or C₁₋₄alkyl (preferably hydrogen) and the —(CH₂)ₛ— group is optionally substituted by hydroxy or C₁₋₄alkyl.

More preferably K is selected from: a direct bond, —(CH₂)ₛ—, —(CH₂)ₛ—O—(CH₂)ₛ—, —(CH₂)ₛ—C(O)—, —C(O)—(CH₂)ₛ—, —(CH₂)ₛ—N(R¹⁴)—, —(CH₂)ₛ—C(O)N(R¹⁴)—, —(CH₂)ₛ—N(R¹⁴)C(O)—(CH₂)ₛ—, —(CH₂)ₛ—S(O)₂N(R¹⁴)— or —(CH₂)ₛ—NHS(O)₂—, wherein s is independently selected from 0, 1, 2, 3 or 4, R¹⁴ is selected from hydrogen or C₁₋₄alkyl (preferably hydrogen or methyl) and the —(CH₂)ₛ— group is optionally substituted by hydroxy or C₁₋₄alkyl.

More preferably K is selected from: a direct bond, methylene, ethylene, propylene, butylene, oxy, 2-hydroxypropylene, carbonyl, methylcarbonyl, ethylcarbonyl, (methyl)methylcarbonyl, (ethyl)methylcarbonyl, carbonylmethylene, carbonylethylene, ethoxyethylene, amino, 2-hydroxypropylamino, carbonylamino, methylcarbonylamino, N-methyl-methylcarbonylamino, aminocarbonyl, methylaminocarbonyl, methylaminocarbonylmethyl, propylsulphonylamino or methylaminosulphonyl.

Further preferably K is selected from: a direct bond, methylene, ethylene, propylene, butylene carbonyl, methylcarbonyl or N-methylmethylcarbonylamino.

Further preferably K is selected from: a direct bond, methyl, carbonyl and methylcarbonyl.

In an particular embodiment, using an alternative representation, K is selected from: a direct bond, —(CH₂)ₛ₁—, —(CH₂)ₛ₁—O—(CH₂)ₛ₂—, —(CH₂)ₛ₁—C(O)—(CH₂)ₛ₂—, —(CH₂)ₛ₁—S(Oₙ)—(CH₂)ₛ₂—, —(CH₂)ₛ₁—N(R¹⁷)—(CH₂)ₛ₂—, —(CH₂)ₛ₁—C(O)N(R¹⁷)—(CH₂)ₛ₂—, (CH₂)ₛ₁—N(R¹⁷)C(O)—(CH₂)ₛ₂—, —(CH₂)ₛ₁—N(R¹⁷)C(O)N(R¹⁷)—(CH₂)ₛ₂—, —(CH₂)ₛ₁—OC(O)—(CH₂)ₛ₂—, —(CH₂)ₛ₁—C(O)O—(CH₂)ₛ₂—, —(CH₂)ₛ₁—N(R¹⁷)C(O)O—(CH₂)ₛ₂—, —(CH₂)ₛ₁—OC(O)N(R¹⁷)—(CH₂)ₛ₂—, —(CH₂)ₛ₁—OS(Oₙ)—(CH₂)ₛ₂—, or —(CH₂)ₛ₁—S(Oₙ)—O—(CH₂)ₛ₂—, —(CH₂)ₛ₁—S(O)₂N(R¹⁷)—(CH₂)ₛ₂— or —(CH₂)ₛ₁—N(R¹⁷)S(O)₂—(CH₂)ₛ₂—; wherein the —(CH₂)ₛ₁— and —(CH₂)ₛ₂— groups are independently optionally substituted by hydroxy or C₁₋₄alkyl group and herein when s1>1 or s2>1 then the CH₂ group can optionally be a branched chain.

For the avoidance of doubt, it should be made clear that where it is stated that a CH₂ group within a —(CH₂)ₛ₁— or —(CH₂)ₛ₂— is di-substituted with C₁₋₄alkyl, it means that both hydrogens within the CH₂ group are replaced by C₁₋₄alkyl groups, such as methyl or ethyl groups. In particular, when the compound of formula (I) includes a group K wherein the —(CH₂)ₛ₁— and —(CH₂)ₛ₂— groups are independently optionally substituted, these are suitably optionally substituted by hydroxy or C₁₋₄alkyl.

Particular examples of groups R¹² include hydroxy, hydroxyC₁₋₆alkyl, oxo, cyano, cyanoC₁₋₆alkyl, nitro, carboxyl, C₁₋₆alkyl, C₁₋₆alkoxy, C₁₋₆alkoxyC₁₋₂alkyl, C₁₋₆alkoxycarbonylC₀₋₂alkyl, C₁₋₆alkanoylC₀₋₂alkyl, C₁₋₆alkanoyloxyC₀₋₂alkyl, C₂₋₆alkenyl, C₁₋₃perfluoroalkyl-, C₁₋₃perfluoroalkoxy, aryl, arylC₁₋₆alkyl, heterocyclyl, heterocyclylC₁₋₆alkyl, N-C₁₋₄alkylaminoC₀₋₂alkyl, N,N-di-C₁₋₄alkylaminoC₀₋₂alkyl, N-C₁₋₄alkylcarbamoylC₀₋₂alkyl, N,N-di-C-₁₋₄alkylaminocarbamoylC₀₋₂alkyl, N-C₁₋₆alkyaminocarbonylC₀₋₂alkyl, N,N-C₁₋₆alkyaminocarbonylC₀₋₂alkyl, C₁₋₆alkyl-S(O)ₙaminoC₀₋₂alkyl-, aryl-S(O)ₙ-aminoC₀₋₂alkyl-, C₁₋₃perfluoroalkyl-S(O)ₙ-aminoC₀₋₂alkyl-; C₁₋₆alkylamino-S(O)ₙ—C₀₋₂alkyl-, arylamino-S(O)ₙ—C₀₋₂alkyl-, C₁₋₃perfluoroalkylamino-S(O)ₙ—C₀₋₂alkyl-, C₁₋₆alkanoylamino-S(O)ₙ—C₀₋₂alkyl-; arylcarbonylamino-S(O)ₙ—C₀₋₂alkyl-, C₁₋₆alkyl-S(O)ₙ—C₀₋₂alkyl-, aryl-S(O)ₙ—C₀₋₂alkyl-, C₁₋₃perfluoroalkyl- or C₁₋₃perfluoroalkoxyC₀₋₂alkyl; wherein an amino group within R¹² is optionally substituted by C₁₋₄alkyl.

For instance, R¹² may be selected from hydroxy, hydroxyC₁₋₆alkyl such as hydroxy methyl or hydroxyethyl, oxo, cyano, cyanoC₁₋₆alkyl such as cyanomethyl or cyanoethyl, nitro, carboxyl, C₁₋₆alkyl such as methyl, ethyl or propyl, C₁₋₆alkoxy such as methoxy or ethoxy, C₁₋₆alkoxyC₁₋₂alkyl such as methoxymethoxy, ethoxymethoxy, ethoxyethoxy or methoxyethoxy, C₁₋₆alkoxycarbonylC₀₋₂alkyl such as methoxycarbonyl or ethoxycarbonyl, C₁₋₆alkanoylC₀₋₂alkyl such as acetyl, C₁₋₃perfluoroalkyl—such as trifluoromethyl, C₁₋₃perfluoroalkoxy such as trifluoromethoxy, aryl such as phenyl, arylC₁₋₆alkyl such as benzyl, N-C₁₋₄alkylaminoC₀₋₂alkyl such as methylamino, N,N-di-C₁₋₄alkylaminoC₀₋₂alkyl such as di-methylamino, N-C₁₋₄alkylcarbamoylC₀₋₂alkyl, such as methylcarbamoyl, or N,N-di-C₁₋₄alkylaminocarbamoylC₀₋₂alkyl such as dimethylcarbamoyl.

Specific examples of R¹² groups include hydroxy, halo such as chloro, cyano, or nitro.

According to a further aspect of the invention there is provided a compound of Formula (Ia) 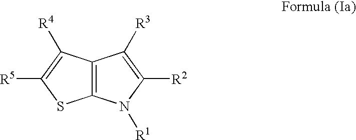
wherein:
R³ is selected from a group of Formula (IIa) or Formula (IIb): 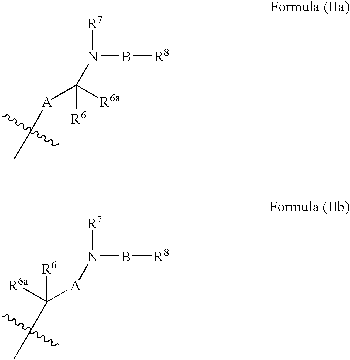
B is a group of Formula (IV) 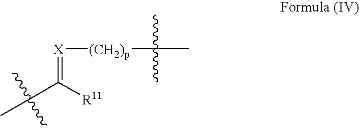
and A, R¹, R², R⁴, R⁵ R⁶, R^{6a}, R⁷, R⁸, and R¹¹ are as defined above for a compound of Formula (I)
   or a salt, solvate or pro-drug thereof.

According to a further aspect of the invention there is provided a compound of Formula (Ib) 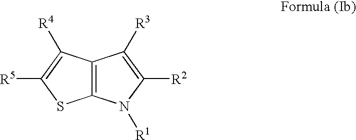
wherein:
R³ is selected from a group of Formula (IIa) or Formula (IIb): 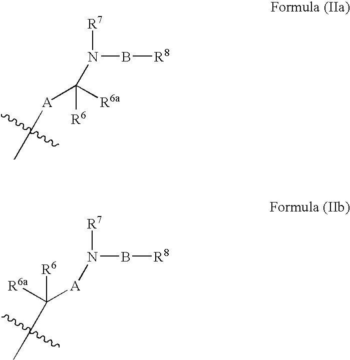
   wherein
the group 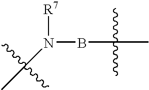
   together forms an optionally substituted heterocyclic ring containing 4-7 carbons atoms, wherein the optional substituents are selected from 1 or 2 substituents independently selected from R¹² and R¹³;
and A, B, R¹, R², R⁴, R⁵ R⁶, R^{6a}, R⁸, R¹² and R¹³ are as defined above for a compound of Formula (I)
   or a salt, solvate or pro-drug thereof.

According to a further aspect of the invention there is provided a compound of Formula (Ic) 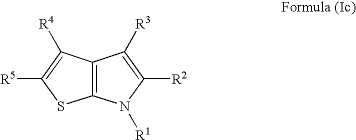
wherein:
R³ is selected from a group of Formula (IIc) or Formula (IId): 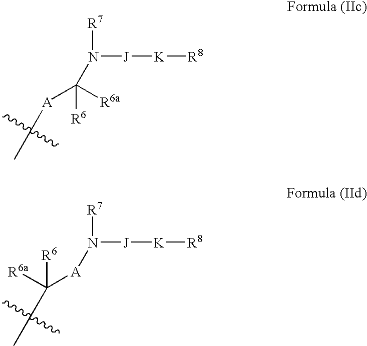
wherein
   the group 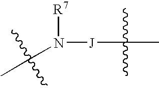
      together forms an optionally substituted heterocyclic ring containing 4-7 carbons atoms, wherein the optional substituents are selected from 1 or 2 substituents independently selected from R¹² and R¹³;
and A, J, R¹, R², R⁴, R⁵ R⁶, R^{6a}, R⁸, and R¹² and R¹³ are as defined above for a compound of Formula (I)
   or a salt, solvate or pro-drug thereof.

According to a further aspect of the invention there is provided a compound of Formula (Id) 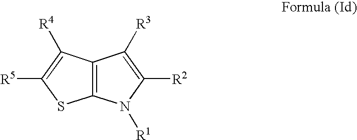
wherein:
R³ is selected from a group of Formula (IIc) or Formula (IId): 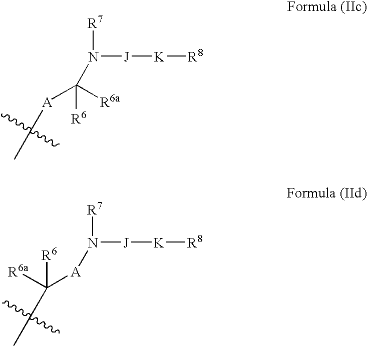
wherein
   J is a group of the formula: —(CH₂)ₛ-L-(CH₂)ₛ— or
   —(CH₂)ₛ—C(O)—(CH₂)ₛ-L-(CH₂)ₛ— wherein when s is greater than 0, the alkylene group is optionally substituted by 1 to 2 group selected from R¹²,
and A, K, L, R¹, R², R⁴, R⁵ R⁶, R^{6a}, R⁸, and R¹² are as defined above for a compound of Formula (I)
   or a salt, solvate or pro-drug thereof.

In particular, in the compound of formula (Id), J is a group of formula —(CH₂)ₛ—C(O)—(CH₂)ₛ-L-(CH₂)—, wherein the compound is a formula of (Id′).

Particular examples of L and s in these cases are described above.

In another particular embodiment, the compound is a compound of formula (Ie) 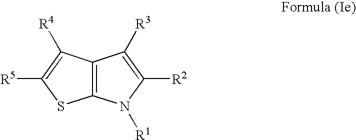
wherein:
R³ is selected from a group of Formula (IIc) or Formula (IId): 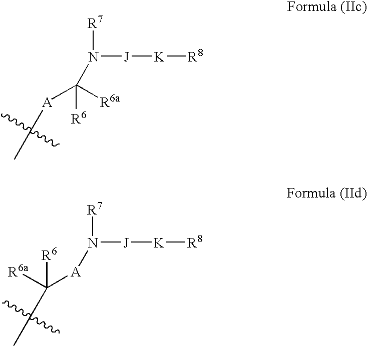
wherein
   K is selected from: —(CR²¹R²²)ₛ₁13 , —(CR²¹R²²)ₛ₁—O—(CR²¹R²²)ₛ₂—, —(CR²¹R²²)ₛ₁—C(O—(CR²¹R²²)ₛ₂—, —(CR²¹R²²)ₛ₁—S(O)ₙ—(CR²¹R²²)ₛ₂—, —(CR²¹R²²)ₛ₁—N(R¹⁷)—(CR²¹R²²)ₛ₂—, —(CR²¹R²²)ₛ₁—C(O)N(R¹⁷)—(CR²¹R²²)ₛ₂—, —(CR²¹R²²)ₛ₁—N(R¹⁷)C(O)—(CR²¹R²²)ₛ₂—, —(CR²¹R²²)ₛ₁—N(R¹⁷)C(O)N(R¹⁷)—(CR²¹R²²)ₛ₂—, —(CR²¹R²²)ₛ₁—OC(O)—(CR²¹R²²)ₛ₂—, —(CR²¹R²²)ₛ₁—C(O)O—(CR²¹R²²)ₛ₂—, —(CR²¹R²²)ₛ₁—N(R¹⁷)C(O)O—(CR²¹R²²)ₛ₂, —(CR²¹R²²)ₛ₁—OC(O)N(R¹⁷)—(CR²¹R²²)ₛ₂—, —(CR²¹R²²)ₛ₁—OS(Oₙ)—(CR²¹R²²)ₛ₂, or —(CR²¹R²²)ₛ₁—S(Oₙ)—O—(CR²¹R²²)ₛ₂—, —(CR²¹R²²)ₛ₁—S(O)₂N(R¹⁷)—(CR²¹R²²)ₛ₂— or —(CR²¹R²²)ₛ₁—N(R¹⁷)S(O)₂—(CR²¹R²²)ₛ₂—; where R¹⁷, n, s1 and s2 are as defined above, and each R²¹ and R²² group is independently selected from hydrogen, hydroxy or optionally substituted C₁₋₄alkyl, wherein the optional substitutent is a group ZR³⁰ where Z is oxygen or a group S(O)ₙ where n is as described above, and R³⁰ is hydrogen or C₁₋₄alkyl, provided that at least one group R²¹ or R²² is a C₁₋₄alkyl substituted by a group ZR³⁰;
      and A, J, L, R¹, R², R⁴, R⁵ R⁶, R^{6a}, R⁸, and R¹² are as defined above for a compound of Formula (I), or a or a salt, solvate or pro-drug thereof.

Preferably in formula (Ie), K is a group —(CR²¹R²²)ₛ₁—C(O)N(R¹⁷)—(CR²¹R²²)ₛ₂—.

Suitably s1 is 0 and s2 is 1 in this case.

In yet a further embodiment, the compound of formula (I) is a compound of formula (If) 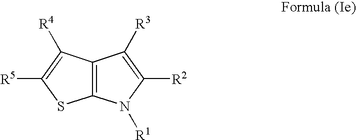
wherein:
R³ is selected from a group of Formula (IIc) or Formula (IId): 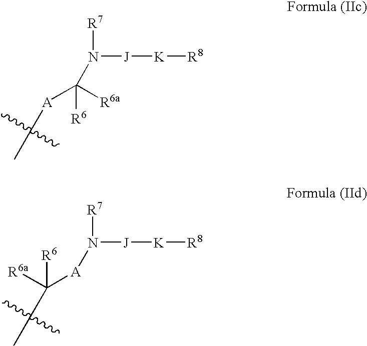
wherein
   K is selected from: —(CR²¹R²²)ₛ₁—, —(CR²¹R²²)ₛ₁—O—(CR²¹R²²)ₛ₂—, —(CR²¹R²²)ₛ₁—C(O—(CR²¹R²²)ₛ₂—, —(CR²¹R²²)ₛ₁—S(O)ₙ—(CR²¹R²²)ₛ₂—, —(CR²¹R²²)ₛ₁—N(R¹⁷)—(CR²¹R²²)ₛ₂—, —(CR²¹R²²)ₛ₁—C(O)N(R¹⁷)—(C²¹R²²)ₛ₂—, —(CR²¹R²²)ₛ₁—N(R¹⁷)C(O)—(CR²¹R²²)ₛ₂—, —(CR²¹R²²)ₛ₁—N(R¹⁷)C(O)N(R¹⁷)—(CR²¹R²²)ₛ₂—, —(CR²¹R²²)ₛ₁—OC(O)—(CR²¹R²²)ₛ₂—, —(CR²¹R²²)ₛ₁—C(O)O—(CR²¹R²²)ₛ₂—, —(CR²¹R²²)ₛ₁—N(R¹⁷)C(O)O—(CR²¹R²²)ₛ₂, —(CR²¹R²²)ₛ₁—OC(O)N(R¹⁷)—(CR²¹R²²)ₛ₂—, —(CR²¹R²²)ₛ₁—OS(Oₙ)—(CR²¹R²²)ₛ₂, or —(CR²¹R²²)ₛ₁—S(Oₙ)—O—(CR²¹R²²)ₛ₂—, —(CR²¹R²²)ₛ₁—S(O)₂N(R¹⁷)—(CR²¹R²²)ₛ₂— or —(CR²¹R²²)ₛ₁—N(R¹⁷)S(O)₂—(CR²¹R²²)ₛ₂—; where R¹⁷, n, s1 and s2 are as defined above, and each R²¹ and R²² group is independently selected from hydrogen, hydroxy or optionally substituted C₁₋₄alkyl, wherein the optional substitutent is a group ZR³⁰ where Z is oxygen or a group S(O)ₙ where n is as described above, and R³⁰ is hydrogen or C₁₋₄alkyl, provided that both group R²¹ and R²² within the same —(CR²¹R²²)ₛ₁13 or —CR²¹R²²)ₛ₂— is a C₁₋₄alkyl group;
   and A, J, L, R¹, R², R⁴, R⁵ R⁶, R^{6a}, R⁸, and R¹² are as defined above for a compound of formula (i), or a or a salt, solvate or pro-drug thereof.

Preferably in this case, the group K is a group —(CR²¹R²²)ₛ₁—C(O—(CR²¹R²²)ₛ₂—.

Suitably in this instance, s1 is 1 and s2 is 0.

According to a further aspect of the invention there is provided a compound of Formula (I), or salt, solvate or pro-drug thereof, wherein R³ is selected from a group of Formula (Ic) or Formula (IId) and R¹, R², R⁴ and R⁵ are as defined above.

According to a further aspect of the invention there is provided a compound of Formula (I), or salt, solvate or pro-drug thereof, wherein R³ is selected from a group of Formula (IIa) or Formula (IIc) and R¹, R², R⁴ and R⁵ are as defined above.

According to a further aspect of the invention there is provided a compound of Formula (I), or salt, solvate or pro-drug thereof, wherein R³ is selected from a group of Formula (IIb) or Formula (IId) and R¹, R², R⁴ and R⁵ are as defined above.

Examples of compounds of formula (I) include:
2-(1,1-dimethyl-2-oxo-2-azabicyclo[2.2.1]heptan-7-ylethyl)-4-[1S-methyl-2-(1-{3-hydroxybenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{3-cyanobenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{3-nitrobenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1-methoxy-2-(4-{pyrrolidin-1-ylcarbonymethyl}-piperazin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1-methoxy-2-(4-{morpholinocarbonyl}-piperazin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1-methoxy-2-(4-{4-methoxypiperidin-1-ylcarbonyl}-piperazin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[2-(4-{1-carbamoyl-3-methylthio-propylaminocarbonyl}-piperidin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[2-(4-{1-carbamoyl-2-methylthio-ethylpropylaminocarbonyl}-piperidin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[2-(4-{morpholinocarbonyl-1,1-dimethylmethylene}-piperidin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{thien-2-ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1{benzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{pyrid-3-ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{benzodioxol-5-ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{3-hydroxybenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{pyrrol-2ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{4-fluorobenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{3-chlorobenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{thien-3-ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{furan-3-ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{cyclohexylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{thiazol-2-ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[2-(4-{1-carbamoyl-2hydroxy-ethylaminocarbonyl}-piperidin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[2-(4-{1-carbamoyl-2hydroxy-propylaminocarbonyl}-piperidin-1-yl)ethyl]-5-(3,5-dimethylphenyl)-6H-2-(1,1-dimethyl-2-oxo-2-azabicyclo[2.2.1]heptan-7-ylethyl)-4-[2-(4-{1-carbamoyl-3-hydroxy-propylaminocarbonyl}-piperidin-1-yl)ethyl]-5-(3,5-dimethylphenyl)-6H-thieno[2,3-b]pyrrole; and
2-(1,1-dimethyl-2-oxo-2-azabicyclo[2.2.1]heptan-7-ylethyl)-4-[2-(4-{3-hydroxypyrrolidin-1-ylcarbonyl-1,1-dimethylmethylene}-piperidin-1-yl)ethyl]-5-(3,5-dimethylphenyl)-6H-thieno[2,3-b]pyrrole;
   or a salt, pro-drug or solvate thereof.

Further examples, include a compound selected from
2-(1,1-dimethyl-2-oxo-2-azabicyclo[2.2.1]heptan-7-ylethyl)-4-[2-(4-{1-carbamoyl-3-methyl-butylaminocarbonyl}-piperidin-1-yl)ethyl]-5-(3,5-dimethylphenyl)-6h-thieno[2,3-b]pyrrole;
2-(1,1-dimethyl-2-oxo-2-azabicyclo[2.2.1]heptan-7-ylethyl)-4-[2-(4-{1-N-methylcarbamoyl-3-methyl-butylaminocarbonyl}-piperidin-1-yl)ethyl]-5-(3,5-dimethylphenyl)-6h-thieno[2,3-b]pyrrole;
2-(1,1-dimethyl-2-oxo-2-azabicyclo[2.2.1]heptan-7-ylethyl)-4-[2-(4-{1-carbamoyl-but-1-ylaminocarbonyl}-piperidin-1-yl)ethyl]-5-(3,5-dimethylphenyl)-6h-thieno[2,3-b]pyrrole;
2-(1,1-dimethyl-2-oxo-2-azabicyclo[2.2.1]heptan-7-ylethyl)-4-[2-(4-{1-carbamoyl-eth-1-ylaminocarbonyl}-piperidin-1-yl)ethyl]-5-(3,5-dimethylphenyl)-6h-thieno[2,3-b]pyrrole;
2-(1,1-dimethyl-2-oxo-2-azabicyclo[2.2.1]heptan-7-ylethyl)-4-[2-(4-{1-carbamoyl-prop-1-ylaminocarbonyl}-piperidin-1-yl)ethyl]-5-(3,5-dimethylphenyl)-6h-thieno[2,3-b]pyrrole; and
2-(1,1-dimethyl-2-oxo-2-azabicyclo[2.2.1]heptan-7-ylethyl)-4-[2-(4-{1-carbamoyl-eth-1-ylaminocarbonyl}-piperidin-1-yl)ethyl]-5-(3,5-dimethylphenyl)-6h-thieno[2,3-b]pyrrole.

A preferred group of compounds according to the present invention are wherein the compound is selected from:
2-(1,1-dimethyl-2-oxo-2-azabicyclo[2.2.1]heptan-7-ylethyl)-4-[1S-methyl-2-(1-{3-hydroxybenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{3-cyanobenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{3-nitrobenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1-methoxy-2-(4-{pyrrolidin-1-ylcarbonylmethyl}-piperazin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1-methoxy-2-(4-{morpholinocarbonyl}-piperazin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1-methoxy-2-(4-{4-methoxypiperidin-1-ylcarbonyl}-piperazin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[2-(4-{1-carbamoyl-3-methylthio-propylaminocarbonyl}-piperidin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[2-(4-{1-carbamoyl-3-methyl-butylaminocarbonyl}-piperidin-1-yl)ethyl]-5-(3,5-dimethylphenyl)-6H-thieno[2,3-b]pyrrole; and
2-(1,1-dimethyl-2-oxo-2-azabicyclo[2.2.1]heptan-7-ylethyl)-4-[2-(4-{morpholinocarbonyl-1,1-dimethylmethylene}-piperidin-1-yl)ethyl]-5-(3,5-dimethylphenyl)-6H-thieno[2,3-b]pyrrole
   or a salt, pro-drug or solvate thereof.

A further preferred group of compounds according to the present invention are wherein the compound is selected from:
2-(1,1-dimethyl-2-oxo-2-azabicyclo[2.2.1]heptan-7-ylethyl)-4-[1-methoxy-2-(4-{pyrrolidin-1-ylcarbonylmethyl}-piperazin-1-yl)ethyl]-5-(3,5-dimethylphenyl)-6H-thieno[2,3-b]pyrrole; and
2-(1,1-dimethyl-2-oxo-2-azabicyclo[2.2.1]heptan-7-ylethyl)-4-[2-(4-{1-carbamoyl-3-methylthio-propylaminocarbonyl}-piperidin-1-yl)ethyl]-5-(3,5-dimethylphenyl)-6H-thieno[2,3-b]pyrrole;
   or a salt, pro-drug or solvate thereof.

The compounds of Formula (I) may be administered in the form of a pro-drug which is broken down in the human or animal body to give a compound of the Formula (I). Examples of pro-drugs include in-vivo hydrolysable esters of a compound of the Formula (I). Various forms of pro-drugs are known in the art. For examples of such pro-drug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

An in-vivo hydrolysable ester of a compound of the Formula (I) containing a carboxy or a hydroxy group is, for example, a pharmaceutically-acceptable ester which is hydrolysed in the human or animal body to produce the parent acid or alcohol. Suitable pharmaceutically-acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example
1-cyclohexylcarbonyloxyethyl; 1,3-dioxolen-2-onylmethyl esters, for example 5-methyl-1,3-dioxolen-2-onlymethyl; and C₁₋₆alkoxycarbonyloxyethyl esters.

An in-vivo hydrolysable ester of a compound of the Formula (I) containing a hydroxy group includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

A suitable pharmaceutically-acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically-acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The compounds of Formula (I) can be prepared by a process comprising a step selected from (a) to (g) as follows, these processes are provided as a further feature of the invention:
(a) Reaction of a compound of formula XXXII with a compound of formula H—R^{3′} to form a compound of Formula (I), 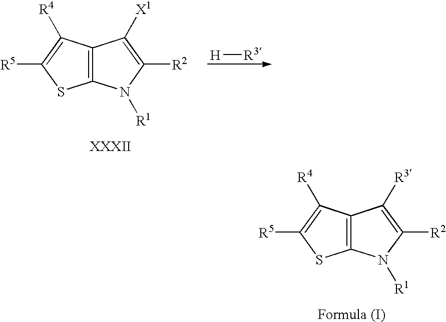
   wherein X¹ is selected from: 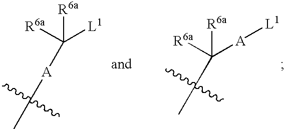
      L¹ is a displaceable group;
   H—R^{3′} is selected from: 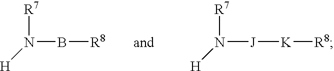
   where, R¹, R², R⁴, R⁵, R⁷, R⁸, B, J, K, A, R⁶ and R^{6a} are as defined above;
(b) Reaction of a compound of formula XXXIII with a compound of formula L²-R^{3″} to form a compound of Formula (I), 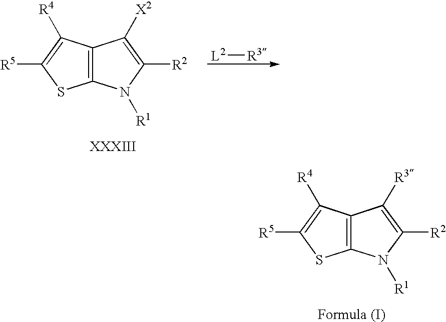
   wherein X² is selected from: 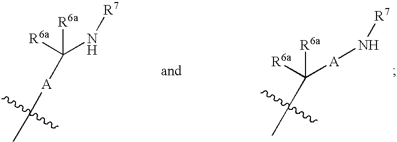
      L² is a displaceable group and R⁷ is selected from the definition of R⁷, and
   L²-R^{3″} is selected from: L²-B—R⁸ and L²-J-K—R⁸,
   where R¹, R², R⁴, R⁵, R⁷, R⁸, B, J, K, A, R⁶ and R^{6a} are as defined above;
(c) For compounds of Formula (I) wherein R³is a group of Formula (IIa), (IIb), (IIc) or (IId) and R⁷ is other than part of a heterocyclic ring or hydrogen, reaction of a compound of Formula (I) wherein R³is a group of Formula (IIa), (IIb), (IIc) or (IId) and R⁷ is hydrogen with a group of formula L³-R^{7a}, wherein R^{7a} is as defined above for R⁷ with the exclusion of hydrogen and L³is a displaceable group;
(d) For compounds of Formula (I) wherein R³ is a group of Formula (IIc) or (IId) and the group 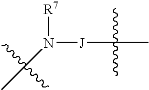
   together forms an optionally substituted nitrogen-containing heterocyclic ring containing 4-7 carbons atoms, reaction of a compound of Formula XXXIVa or XXXIVb, with a compound of Formula L⁶-K—R⁸, wherein L⁶ is a displaceable group 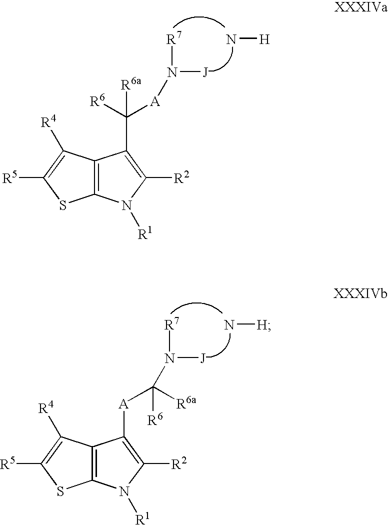
   where R¹, R², R⁴, R⁵, R⁷, R⁸, J, K, A, R⁶ and R^{6a} are as defined above;
(e) For compounds of Formula (I) wherein R³ is a group of Formula (IIc) or (IId), reaction of a compound of Formula XXXVa or XXXVb, with a compound of Formula L⁷-K″—R⁸, wherein L⁷ is a displaceable group, and wherein the groups K′ and K″ comprise groups which when reacted together form K, 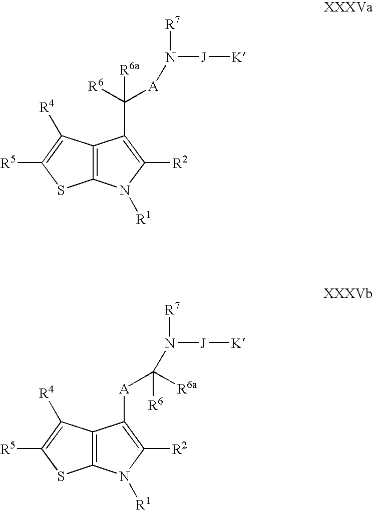
   where R¹, R², R⁴, R⁵, R⁷, R⁸, J, K, A, R⁶ and R^{6a} are as defined above;
(f) reaction of a compound of Formula XXXVI with an electrophilic compound of the formula L⁸-R³, wherein L⁸ is a displaceable group 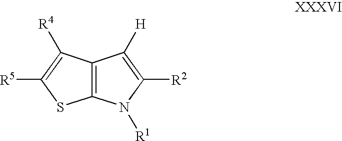
   where R¹, R², R³, R⁴ and R⁵ are as defined above;
(g) reaction of a compound of Formula XXXVII with a compound of the formula L¹⁰-R², wherein L⁹ is a leaving group and L¹⁰ is an activating group or L⁹ is an activating group and L¹⁰ is a leaving group 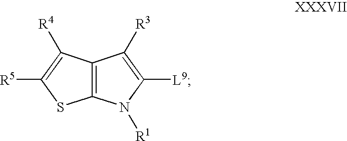
   where R¹, R², R³, R⁴and R⁵ are as defined above;
      and thereafter if necessary:
i) converting a compound of the Formula (I) into another compound of the Formula (I);
ii) removing any protecting groups;
iii) forming a salt, pro-drug or solvate.

Specific reaction conditions for the above reations are as follows:
Process a) Compounds of formula XXXII and H—R^{3′} can be coupled together in the presence of an organic base (such as DIPEA [di-isopropylethylamine]) or an inorganic base (such as potassium carbonate) base, in a suitable solvent such as DMA or DMF, at a temperature from room temperature and 120° C. Suitable displaceable groups include: a halide, such as chloro, or a methane sulphonate or toluene sulphonate;
Process b) Compounds of XXXIII and L²-R^{3″} can be coupled together in the presence of an organic base(such as DIPEA) or an inorganic base (such as potassium carbonate), in a suitable solvent such as DMA or DMF, at a temperature from room temperature to 120° C. Suitable displaceable groups include: a halide, such as chloro, or a methane sulphonate or toluene sulphonate,
   alternatively if L² is a hydroxy group then the L²-R^{3″} ; can be reacted with a compound of formula XXXIII under Mitsunobu reaction conditions;
Process c and d) Reaction conditions to facilitate these reactions can be using (i) alkylation reaction conditions or (ii) acylation reaction conditions: Examples of said conditions include:
   (i) alkylation reaction conditions—the presence of an organic base(such as DIPEA) or an inorganic base (such as potassium carbonate), in a suitable solvent such as DMF, DMA, DCM, at a temperature from room temperature to 120° C. Suitable displaceable groups include: a halide, such as chloro, methane sulphonate or toluene sulphonate;
   (ii) acylation reaction conditions—presence of organic base, such as triethylamine, temperature 0° C. to 50-60° C. in a suitable solvent such as DCM. Suitable displaceable groups include an acylchloride or an acid anhydride,
process e) The skilled man would be familiar with a variety of reaction conditions and values for K′ and K″, which when reacted together would form the group K, examples of aid conditions and values for K′ and K″ include:
   (i) For compounds of Formula (I) where K is —(CH₂)ₛ₁—N(R¹⁷)C(O)—(CH₂)ₛ₂— these can be prepared by reacting a compound where K′ is —(CH₂)ₛ₁—N(R¹⁷)H with a carboxylic acid for formula HOOC—(CH₂)ₛ₂—R⁸ to form the amide. Coupling of amino groups with carboxylic acids are well known in the art and can be facilitated by a number of chemical reactions using an appropriate coupling reagent. For example a carbodiimide coupling reaction can be performed with EDCl in the presence of DMAP in a suitable solvent such as DCM, chloroform or DMF at room temperature;
   (ii) For compounds of Formula (I) where K is —(CH₂)ₛ₁—C(O)N(R¹⁷)—(CH₂)ₛ₂— these can be prepared by reacting a compound where K′ is —(CH₂)ₛ₁—COOH with an amine of the HN(R¹⁷)—(CH₂)ₛ₂—R⁸ to form the amide. Methodology is identical to processes described in (i) above in this section;
   (iii) For compounds of Formula (I) where K is —(CH₂)ₛ₁—N(R¹⁷)C(O)O—(CH₂)ₛ₂— these can be prepared by reacting a compound where K′ is —(CH₂)ₛ₁—N(R¹⁷)H with a chloroformate of formula ClC(O)O—(CH₂)ₛ₂—R⁸ in a suitable solvent, such as DCM or chloroform, in the presence of a base, such as N-methylmorpholine, pyridine or triethylamine, at a temperature between −10° C. and 0° C.;
   (iv) For compounds of Formula (I) where K is —(CH₂)ₛ₁—OC(O)N(R¹⁷)—(CH₂)ₛ₂— these can be prepared by reacting a compound where K′ is —(CH₂)ₛ₁—OC(O)Cl with a compound of formula HN(R¹⁷)—(CH₂)ₛ₂—R⁸. Methodology is identical to processes described in (iii) above in this section;
   (v) For compounds of Formula (I) where K is —(CH₂)ₛ₁—N(R¹⁷)S(O₂)—(CH₂)ₛ₂— these can be prepared by reacting a compound where K′ is —(CH₂)ₛ₁—N(R¹⁷)H with a sulphonyl chloride of formula ClS(O₂)—(CH₂)ₛ₂—R⁸ in the presence of a base, such as triethylamine or pyridine, in a suitable solvent such as chloroform or DCM at a temperature between 0° C. and room temperature;
   (vi) For compounds of Formula (I) where K is —(CH₂)ₛ₁—S(O₂)N(R¹⁷)—(CH₂)ₛ₂— these can be prepared by reacting a compound where K′ is —(CH₂)ₛ₁—S(O₂)Cl with a compound of HN(R¹⁷)—(CH₂)ₛ₂—R⁸. Methodology is identical to processes described in (v) above in this section
   (vii) For compounds of Formula (I) where K is —(CH₂)ₛ₁—N(R¹⁷)—(CH₂)ₛ₂— these can be prepared by reacting a compound where K′ is —(CH₂)ₛ₁-L¹¹ with a compound of formula HN(R¹⁷)—(CH₂)ₛ₂—R⁸, wherein L¹¹ is a displaceable group. This reaction can be performed in the presence of an organic base(such as DIPEA) or an inorganic base (such as potassium carbonate), in a suitable solvent such as DMA or DMF, at a temperature from room temperature to 120° C. Suitable displaceable groups include: a halide, such as chloro, or a methane sulphonate or toluene sulphonate. Compounds can also be prepared by reacting a compound wherein K′ is —(CH₂)ₛ₁—N(R¹⁷)H with a compound of formula L¹¹-(CH₂)ₛ₂—R⁸, under identical conditions.
   (viii) For compounds of Formula (I) where K is —(CH₂)ₛ₁—O—(CH₂)ₛ₂— these can be prepared by reacting a compound where K′ is —(CH₂)ₛ₁—OH with a compound of formula L¹²-(CH₂)ₛ₂—R⁸, wherein L¹² is a displaceable group. This reaction can be performed in the presence of an organic base (such as potassium t-butoxide) or an inorganic base (such as sodium hydride), in a suitable solvent such as DMA or DMF, at a temperature from room temperature to 120° C. Suitable displaceable groups include: a halide, such as bromo, or a methane sulphonate or toluene sulphonate. Compounds can also be prepared by reacting a compound wherein K′ is —(CH₂)ₛ₁-L¹² with a compound of formula HO—(CH₂)ₛ₂—R⁸, under identical conditions.
   (ix) For compounds of Formula (I) where K is —(CH₂)ₛ₁—C(O)—(CH₂)ₛ₂— these can be prepared by reacting a compound where K′ is —(CH₂)ₛ₁—C(O)-L¹³ with a Grignard reagent of formula BrMg(CH₂)ₛ₂—R⁸, wherein L¹³ is a displaceable group. This reaction can be performed in a non-polar solvent such as THF or diethylether at a temperature between room temperature and the boiling point of the solvent. Suitable displaceable groups include: a halide, such as chloro, or an alkoxide. Compounds can also be prepared by reacting a compound wherein K′ is —(CH₂)ₛ₁—MgBr with a compound of formula L¹³-C(O)—(CH₂)ₛ₂—R, under identical conditions.
Process f) reaction of a compound of Formula XXXVI with a compound of the formula L⁸-R³, can be performed under Friedel Craft conditions, for example in the presence of diethylaluminium chloride in a suitable solvent, such as DCM, in an inert atmosphere such as nitrogen, at a temperature between room temperature and the boiling point of the solvent or under Mannich conditions, for example, formaldehyde and a primary or secondary amine in acetic acid, in an inert atmosphere such as nitrogen at a temperature between room temperature and 100° C.
Process g) reaction of a compound of Formula XXXVII with a compound of the formula L¹⁰-R², wherein L⁹ is a leaving group and L¹⁰ is an activating group or L⁹ is an activating group and L¹⁰ is a leaving group, can be performed in an aprotic, polar solvent such as THF, using palladium chemistry under Suzuki or Stille conditions, at a temperature between 0 to 70° C.

It will be appreciated by those skilled in the art that in the processes of the present invention certain functional groups such as hydroxyl or amino groups in the starting reagents or intermediate compounds may need to be protected by protecting groups. Thus, the preparation of the compounds of Formula (I) may involve, at an appropriate stage, the addition and subsequent removal of one or more protecting groups.

The protection and de-protection of functional groups is described in ‘Protective Groups in Organic Chemistry’, edited by J. W. F. McOmie, Plenum Press (1973) and ‘Protective Groups in Organic Synthesis’, 2nd edition, T. W. Greene and P. G. M. Wuts, Wiley-Interscience (1991).

A suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The de-protection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium-on-carbon.

### EXPERIMENTAL

General Reaction Schemes

In the following schemes wherein Ri, Rii and Riii represent optional substituents on the phenyl ring which are optionally protected as necessary and R represents a protecting group, group C has been depicted as substituted phenyl for illustration purposes only. Other definitions of C are also appropriate.

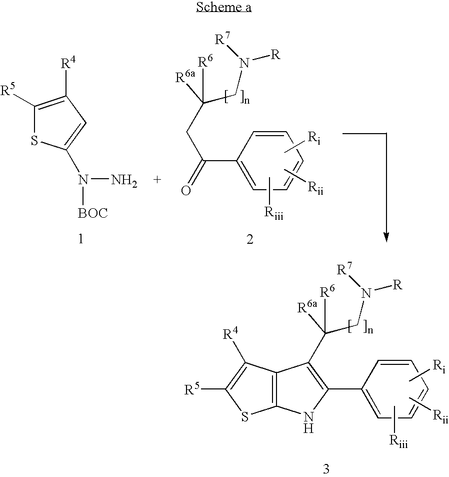

Thienopyrroles, such as 3 can be synthesised by the classic Fisher thienopyrrole synthesis reaction by the condensation of a hydrazine-HCl 1 and a ketone 2, bearing hydrogen atoms α to the carbonyl (Scheme a). Treatment of these reactants in a suitable solvent, such as acetic acid, ethanol, sec-butanol, toluene, in the presence of an acid, such as sulphuric, hydrochloric, polyphosphoric and/or a Lewis acid, for example, boron trifluoride, zinc chloride, magnesium bromide, at elevated temperatures (for example 100° C.), gives the desired product. R represents a protecting group, eg tert-butylcarbamate or phthalimide.

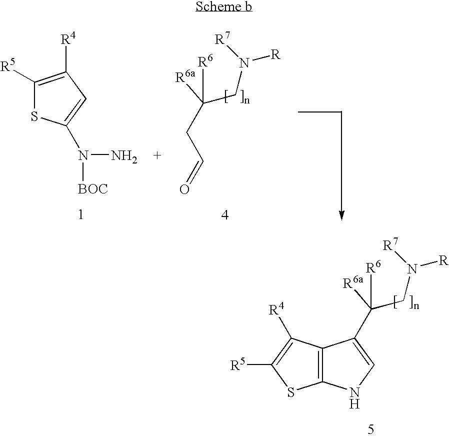

Thienopyrroles, such as represented in structure 5, can also be made using aldehydes 4, bearing hydrogen atoms α to the carbonyl, by cyclization using the conditions above. In this case the substituent at the 2-position must be added later (see scheme d).

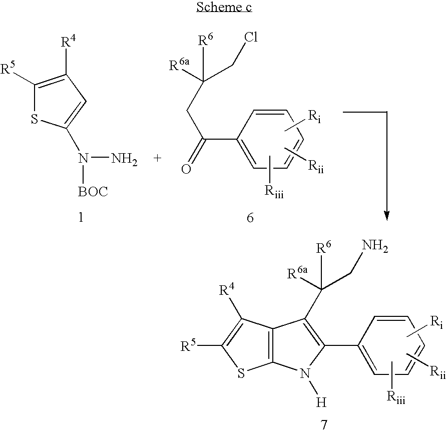

Thienopyrrole may also be synthesised utilising the Granburg reaction, wherein a hydrazine 1 is mixed with ketone 6, bearing a chlorine atom γ to the carbonyl, and heated in a suitable solvent such as ethanol, sec-butanol, toluene at a temperature between 50° C. and 120° C. (Scheme c).

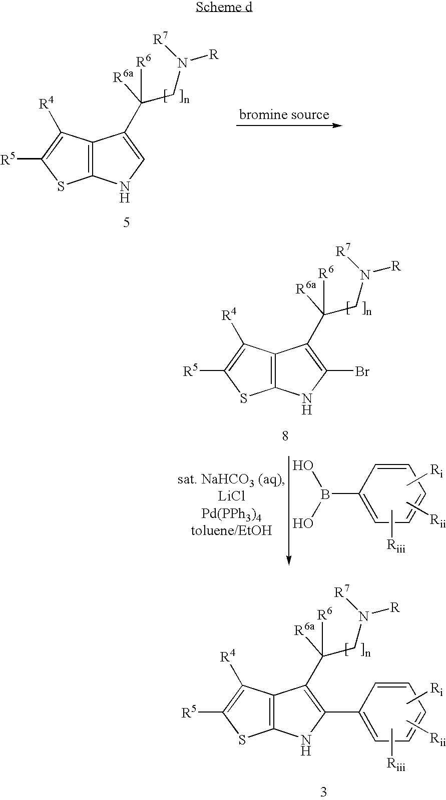

The thienopyrrole 5 can be treated with a ‘bromine source’, such as molecular bromide, pyridinium tribromide, pyrrolidone hydrobromide or polymer supported reagent equivalents, in an inert solvent such as chloroform, methylene chloride at −10° C. to 25° C. to yield the 2-bromo compound 8 (Scheme d). Reaction under Suzuki conditions with a palladium(0) catalyst, a weak base such as aqueous sodium carbonate or saturated sodium hydrogen carbonate and the like, and a substituted aryl boronic acid from commercial sources or prepared (as described in: Gronowitz, S.; Hornfeldt, A.-B.; Yang, Y.,-H Chem. Sci. 1986, 26, 311-314), in an inert solvent such as toluene, benzene, dioxane, THF, DMF and the like, with heating between 25° C. and 100° C., preferably 80° C., for a period of 1-12 hours, to give the desired compound 3.

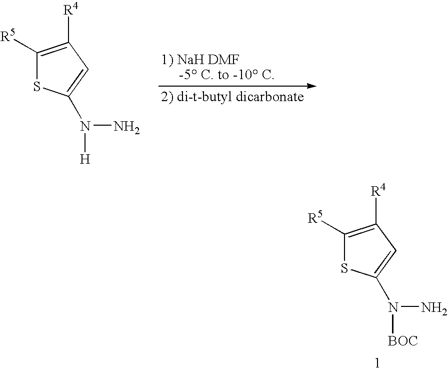

The thiophene 1 can be synthesised by reaction of a hydrazine under the preferred conditions of sodium hydride in DMF at a temperature between −10° C. and −5° C., followed by reaction with di-tert-butyldicarbonate in THF under reflux.

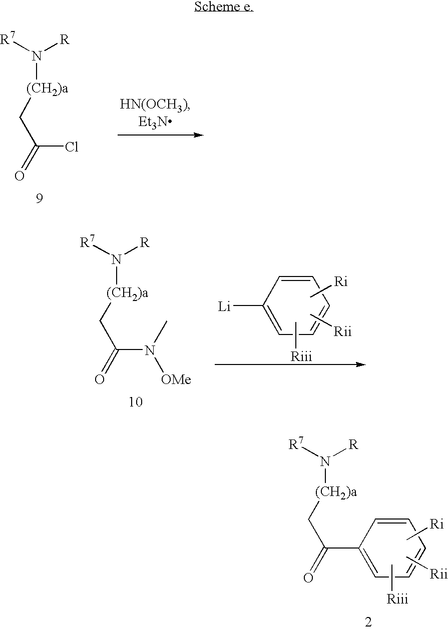

Substituted ketones 2 can be prepared, as outlined in Scheme e starting from appropriate acid chlorides such as 9. Treatment of the acid chloride with N,N-dimethylhydroxylamine hydrochloride in the presence of an amine base such as triethylamine, and a suitable solvent such as methylene chloride at a temperature of −10° C. to 25° C., yields the amide 10. Further reaction with a substituted aryl organolithium (prepared essentially as described in Wakefield B, J.; Organolithium Methods Academic Press Limited, 1988, pp. 27-29 and references therein) in an inert solvent such as tetrahydrofuran, diethyl ether, benzene, toluene or mixture thereof and the like, at a temperature between −100° C. and 0° C. then quenching of the reaction mixture with a mineral acid such as hydrochloric acid, yields the aryl ketone 2.

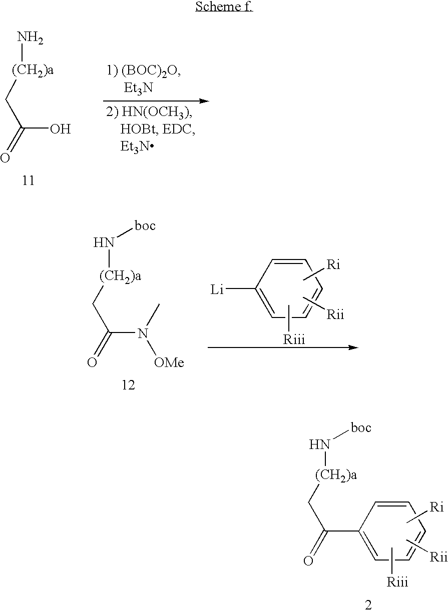

Commencing with a readily available amino acid with a suitable chain length [a] 11, the nitrogen atom can be brought in at the beginning of the synthesis by the route shown in Scheme f. Protection of the amine group of 11 with a tert-butylcarbamate group is achieved by condensation with di-tert-butyl di-carbonate in the presence of an amine base, for example triethylamine, in an inert solvent such as methylene chloride, chloroform, benzene, toluene, tetrahydrofuran and mixtures thereof and the like, at a temperature of −10° C. to 25° C. Coupling of the acid product with N,N-dimethylhydroxylamine in the presence of a coupling reagent 1-(3-dimethylaminopropyl)-3-ethylcarbodiimide hydrochloride (EDC) or 1,3-dicyclohexylcarbodiimide (DCC) or the like, with or without 1-hydroxybenzotriazole (HOBt), and suitable amine base, such as triethylamine and the like, in an inert solvent such as methylene chloride, chloroform, dimethylformamide, or mixture thereof, at or near room temperature for a period of 3 to 24 hours provided the corresponding coupled product 12. Following the same route described above for scheme e, the aryl group can then be installed.

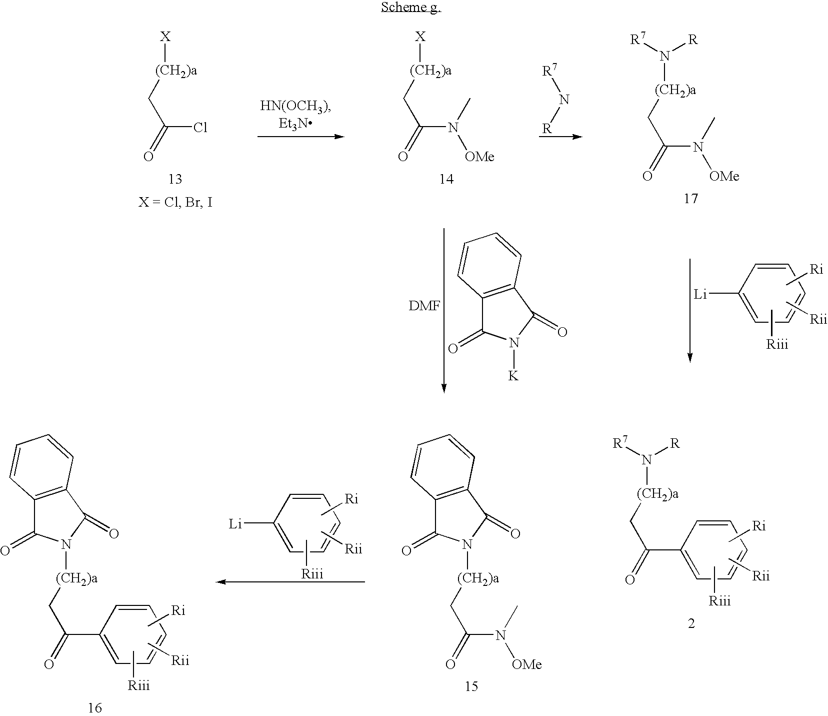

Scheme g illustrates another method for the synthesis of ketone such as 2 and 16, where the nitrogen group is introduced at a latter stage. As above a Weinreb amide 14 can be synthesised from an acid chloride. Treatment with the required amine, in an inert solvent such as THF, toluene, water and the such like can displace the group X to give 17. As above the aryl group can be introduced by displacement of the Weinreb amide with a suitable aryl lithium nucleophile. Alternatively the nitrogen atom can be introduced already protected as a phthalimide by displacement of the group X by potassium phthalimide, or similar salt thereof, by heating in an inert polar solvent such as DMF, DMSO, THF, toluene with or without the presence of a catalyst such as tetrabutylammonium iodide and the such like, to yield the compound 15. Again displacement of the Weinreb amide with an organolithium species completes the synthesis of ketone 16 suitable for cyclization under the Fischer condition described above for thienopyrrole synthesis.

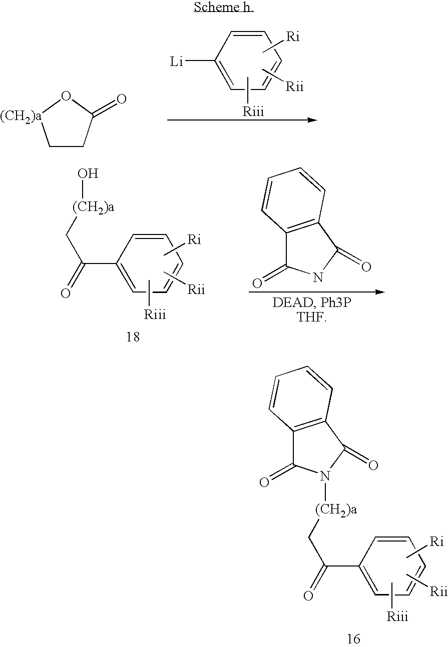

An alternative approach to a phthalimide protected nitrogen ketone, such as 16, can be taken by firstly treating a lactone, with an organolithium species as in the above schemes in a suitable solvent such as THF or ether at a low temperature of between −100° C. and −50° C. to yield a primary alcohol 18 (Scheme h). The hydroxyl function of 18 is replaced with a phthalimide group by a Mitsunobu reaction with an activating agent such as diethyldiazocarboxylate (DEAD), diisopropyldiazocarboxylate or the like with triphenylphosphine, tri-butylphosphine and the like, in an inert solvent such as benzene, toluene, tetrahydrofuran or mixtures thereof to give the desired ketone 16.

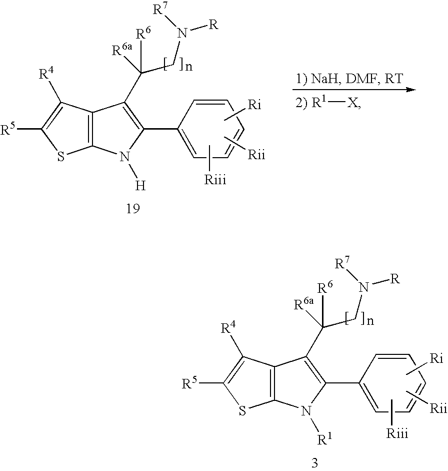

If the group R¹ was not present on the starting hydrazine before cyclization to form a thienopyrrole it may be added post cyclization by an alkylation reaction (19→3). The thienopyrrole is de-protonated by a strong base, such as sodium hydride, n-butyl lithium, lithium diisopropylamine, sodium hydroxide, potassium tert-butoxide in a suitable inert solvent such as THF, DMF, DMSO and the such like, and an alkyl halide added and the mixture stirred at room temperature.

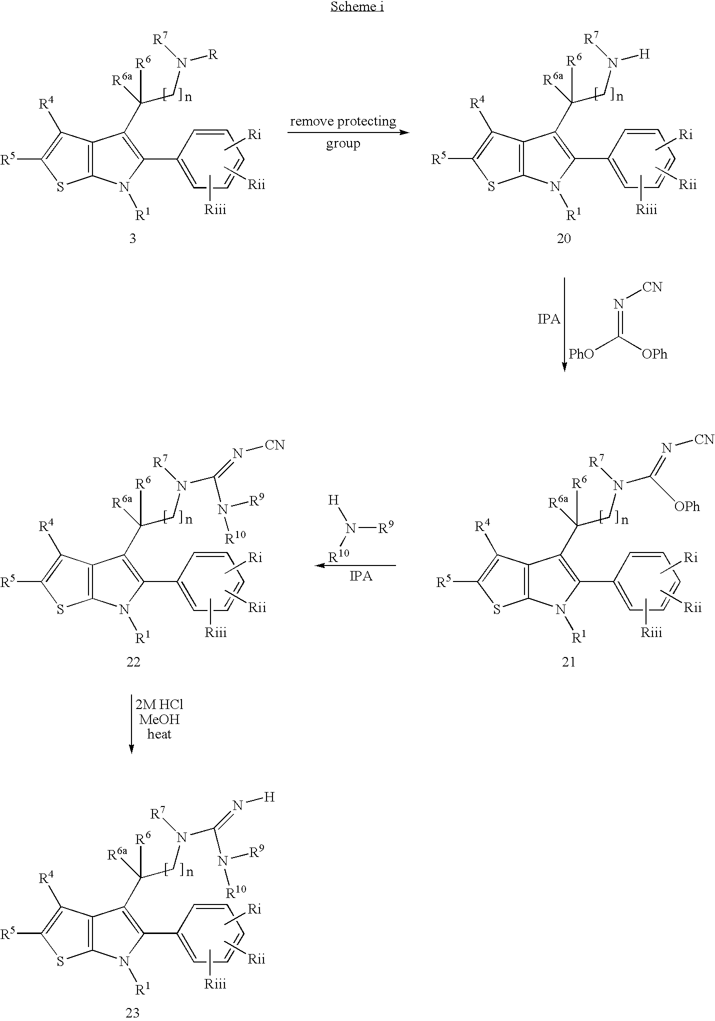

Depending on the route used above a thienopyrrole 20 suitable for conversion to a cyano-guanidine can be formed by removal of the protecting group, for example if a tert-butylcarbamate group was used then removal is accomplished using a strong acid, for example trifluoroacetic acid or hydrochloric acid in an inert solvent such as methylene chloride, chloroform, THF or dioxane at a temperature between −20° C. and 25° C. A phthalimide group, for example, can be removed by hydrazine in a suitable solvent for example methanol, ethanol, methylene chloride, chloroform, THF dioxane at a temperature between −20° C. and 25° C. The primary amine 20 can be converted to a cyano-guanidine 22 by the two step process of reaction with diphenyl cyanocarbonimidate in an inert organic solvent such as iso-propyl alcohol, methylene chloride, chloroform, benzene, tetrahydrofuran and the like, at a temperature between −20° C. and 50° C., followed by condensation with an appropriately substituted amine in an inert organic from the list above, with heating at a temperature between −20° C. and 100° C. (Scheme i 20→21→22). Further treatment of 22 with 2 molar Hydrochloric acid in methanol at elevated temperature yields guanidine compounds 23.

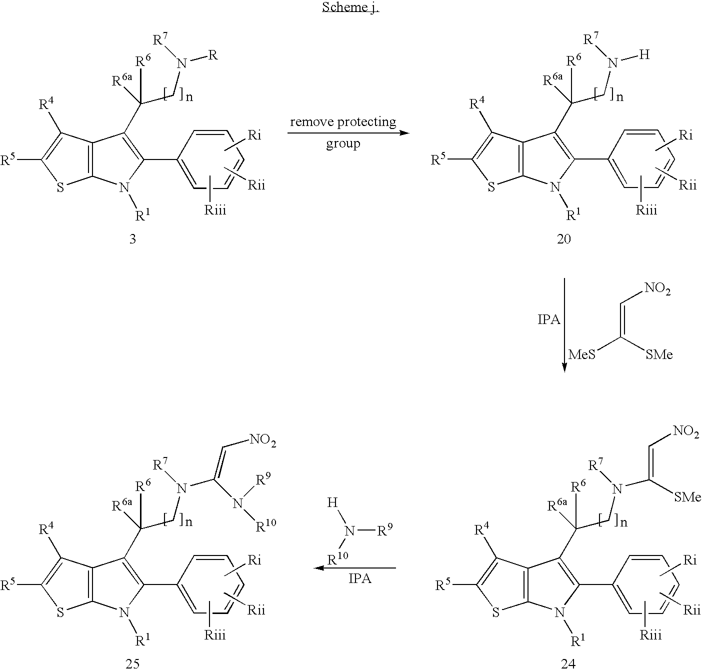

Similarly, reaction with 1,1′-bis(methylthio)-2-nitroethylene in an inert solvent such methylene chloride, chloroform, benzene, tetrahydrofuran and the like, followed by condensation with an appropriately substituted amine in an inert organic solvent from the list above yields the nitroethyleneimidazo[1,2-a]pyridine 25 (Scheme j, 20→24→25).

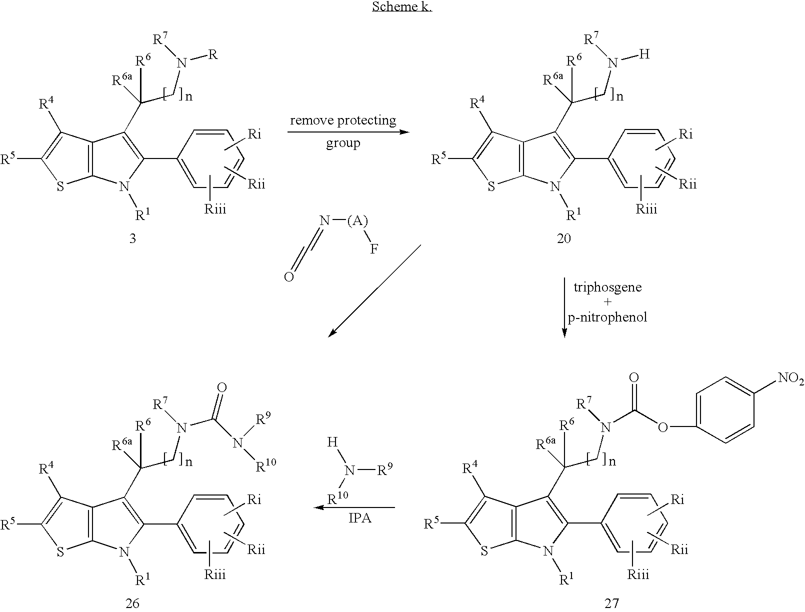

Again in a similar fashion the suitable thienopyrrole 20, derived from de-protection, can be converted to a urea by either direct treatment with an iso-cyanate in an inert solvent such as methylene chloride, chloroform or THF and the such like, or by a two step procedure of reaction with triphosgene (20→27) followed by addition of an amine (27→26), bearing the required substitution to yield 26.

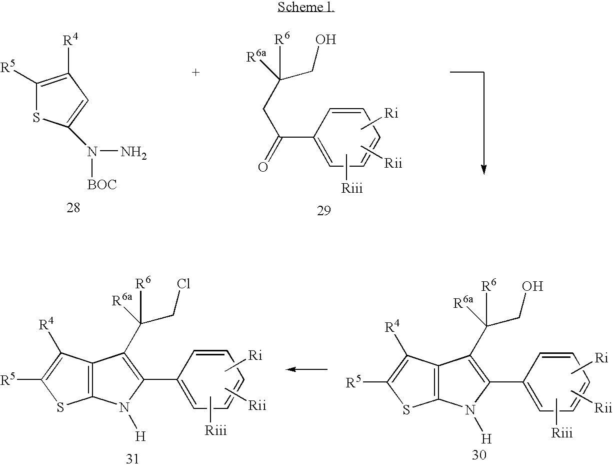

Chloro thieno-pyrrole intermediates, such as 31, can be made as shown in Scheme 1. 30 can synthesized by the classic Fisher thieno-pyrrole synthesis reaction by the condensation of a hydrazine-HCl 28 and a ketone 29, bearing hydrogen atoms α to the carbonyl. Treatment of these reactants in a suitable solvent, such as acetic acid, ethanol, sec-butanol, toluene, in the presence of an acid, such as sulphuric, hydrochloric, polyphosphoric and/or a Lewis acid, for example, boron trifluoride, zinc chloride, magnesium bromide, at elevated temperatures (for example 100° C.), gives the desired product. The chloro intermediate 31 can then be synthesized from 30 using, for example, either (i) sulphonyl chloride in methylene chloride at a temperature of about 0° C., or (ii) CCl₄ followed by triphenylphosphine in a solvent such as acetonitrile at a temperature of about 0° C. Thienopyrroles of the invention can then be prepared by displacement of chlorine atom using an appropriate side chain intermediate such as a substituted heterocyclic ring.

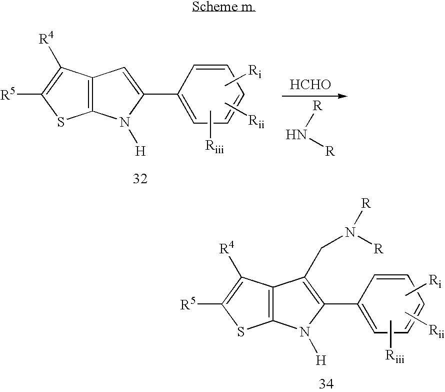

Thienopyrroles of Formula (I) wherein A is a direct bond and R⁶ and R^{6a} are both hydrogen can be prepared as shown in Scheme m. A thieno-pyrrole 32 can be reacted with formaldehyde and an amine, in a suitable solvent such as acetic acid/dioxan at a temperature of about 0° C. to 25° C. for between about 1 to 8 hours, form the thieno-pyrrole 34.

### EXAMPLES

The invention will now be illustrated with the following non-limiting examples in which, unless otherwise stated:

(i) evaporations were carried out by rotary evaporation in vacuo and work-up procedures were carried out after removal of residual solids such as drying agents by filtration;

(ii) operations were carried out at room temperature, that is in the range 18-25° C. and under an atmosphere of an inert gas such as argon or nitrogen;

(iii) yields are given for illustration only and are not necessarily the maximum attainable;

(iv) the structures of the end-products of the Formula (I) were confirmed by nuclear (generally proton) magnetic resonance (NMR) and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet;

(v) intermediates were not generally fully characterised and purity was assessed by thin layer chromatography (TLC), high-performance liquid chromatography (HPLC), infra-red (IR) or NMR analysis;

(vi) chromatography was performed on silica (Merck Keiselgel: Art.9385);

(vii) isolute™ refers to silica (SiO₂) based columns with irregular particles with an average size of 50 μm with nominal 60 Å porosity [Source: Jones Chromatography, Ltd., Glamorgan, Wales, United Kingdom].

Abbreviations

| | |
|---|---|
| DCC | 1,3-dicyclohexylcarbodiimide |
| DCM | dichloromethane |
| DEAD | diethylazodicarboxylate |
| DIPEA | di-isopropylethylamine |
| DMA | dimethylacetamide |
| DMSO | dimethyl sulphoxide |
| DMAP | 4-dimethylaminopyridine |
| DMF | dimethylformamide |
| DTBAD | |
| EDCI | 1-(3-dimethylaminopropyl)-3-ethylcarbodiimide |
| | hydrochloride |
| HATU | (O-(7-azabenzotriazol-1-yl)-N,N,N′,N′- |
| | tetramethyluronium hexafluorophosphate) |
| HOBt | 1-hydroxybenzotriazole |
| THF | tetrahydrofuran |

### Example 1

### 2-(1,1-dimethyl-2-oxo-2-azabicyclo[2.2.1]heptan-7-ylethyl)-4-[1S-methyl-2-(1-{thien-2-ylmethyl}-azetidin-3-ylcarbonylamino)ethyl]-5-(3,5-dimethylphenyl)-6H-thieno[2,3-b]pyrrole

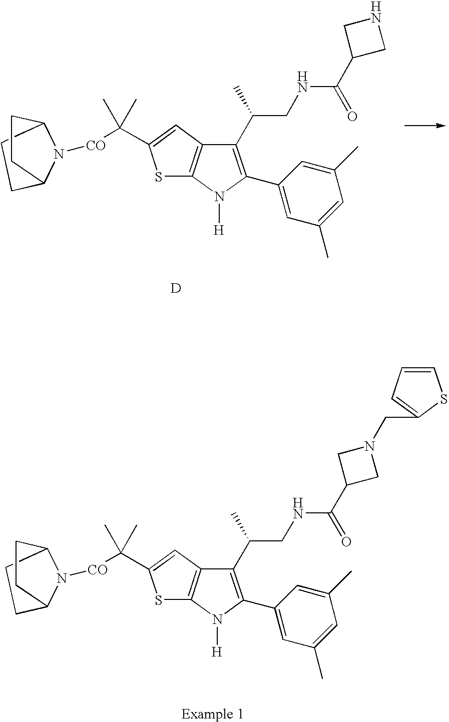

To a solution of D (75 mg; 0.14 mmol) in methanol (2 ml) were added at room temperature the 2-formyl-thiophene (0.17 mmol, 1.2 equiv.), then cyanosodium borohydride (13 mg; 0.21 mmol, 1.5 equiv.) and acetic acid (100 μl, catalytic) and the reaction mixture was stirred overnight. Methanol was removed and the mixture was dissolved into 1 ml DMA, then purified using a reverse phase C₁₈ column, Xterra Waters, 19*100 mm, 5 μ (gradient: 0 to 100% MeCN in water containing 2 g/l ammonium carbonate). The residue was concentrated and precipitated in a mixture pentane/diethyl ether to give Example 1 as a solid after filtration.

Yield: 23-62% ¹H NMR spectrum (DMSO-d₆+TFA-d): mixture of rotamers 1.23 (d, 3H); 1.25-1.38 (m, 4H); 1.43-1.58 (m, 4H); 1.52 (br s, 6H); 2.30 (s, 2.4H); 2.32 (3.6H); 3.12-3.27 (m, 2H); 3.36-3.56 (m, 2H); 3.78-7.21 (m, 5H); 4.40-4.56 (m, 1H); 4.60 (br s, 2H); 6.75 (br s, 1H); 6.89 (s, 0.4H); 6.93 (0.6H); 7.06 (s, 0.8H), 7.09 (s, 1.2): 7.13 (br s, 1H); 7.29 (s, 0.6H); 7.30 (s, 0.4H); 7.68 (s, 0.6H); 7.69 (s, 0.4H); 8.25 (br s, 1H); 11.30 (br s, 1H). MS-ESI: [M+H]⁺ 629

The starting material D was prepared according to the following scheme.

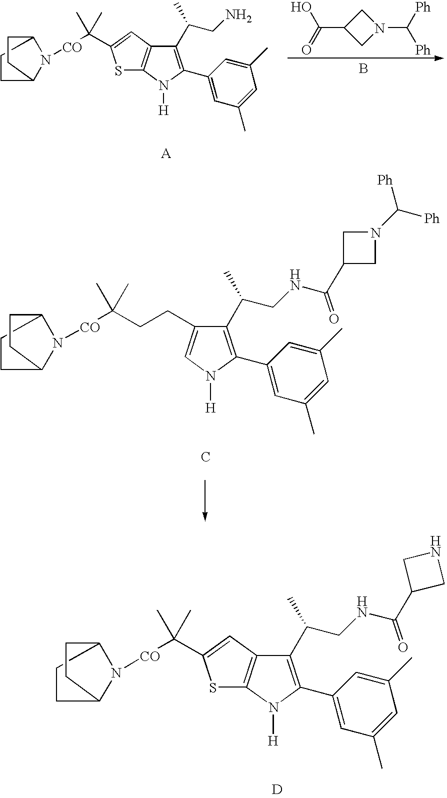

To a solution of A (3 g; 6.67 mmol) in DMF (10 ml) were successively added at 0° C. diisopropylethylamine (1.74 ml; 10 mmol, 1.5 equiv.), B (2.14 g; 8 mmol, 1.2 equiv.) and HATU (3.04 g; 8 mmol, 1.2 equiv.). The mixture was stirred over a week end. After removal of DMF under reduced pressure, a saturated aqueous solution of NaHCO₃ was added and extracted three times with dichloromethane. The combined organic layers were washed with brine, dried over MgSO₄. The solvent was evaporated and the residue purified by flash chromatography eluting with 80/20 ethyl acetate/petroleum ether to give C (4.1 g).

Yield: 88% ¹H NMR spectrum (DMSO-d₆): 1.16 (d, 3H); 1.20-1.35 (m, 4H); 1.40-1.55 (m, 5H); 1.49 (s, 6H); 2.26 (s, 6H); 2.93-3.34 (m, 6H); 3.40 (m, 1H); 4.05 (s br, 1H); 4.36 (s, 1H); 4.50 (s br, 1H); 6.74 (s, 1H); 6.85 (s, 1H); 7.04 (s, 2H); 7.17 (m, 2H); 7.25 (m, 4H); 7.38 (m, 4H); 7.83 (m, 1H); 11.23 (s, 1H). MS-ESI: 699 [M+H]⁺

To a solution of C (4.1 g; 5.87 mmol) in dichloromethane (20 ml) was added at 0° C. 1-chloroethyl chloroformate (1.21 ml; 11.74 mmol, 2 equiv.). 1,8-diamino naphthalene (0.93 g; 5.87 mmol, 1 equiv.) was added after two hours and the reaction mixture was stirred overnight. Methanol was added (20 ml) and the mixture was heated under reflux for 45 minutes. After removal of solvents under reduced pressure, a saturated aqueous solution of NaHCO₃ was added and extracted four times with dichloromethane. The combined organic layers were washed with brine, dried over MgSO₄. The solvent was evaporated and the residue purified by flash chromatography eluting with increasingly polar mixtures of NH₃ 3.5 M in MeOH/CH₂Cl₂ (5-10% NH₃—MeOH) to give D (1.2 g) which was used without any further characterization into next step.

Yield: 38% MS-ESI: 533 [M+H]⁺

Using a method analogous to the preparation of Example 1, the following compounds were prepared by reaction of D with the corresponding aldehyde:

| | STRUCTURE | MS-ESI: [M + H]⁺ |
|---|---|---|
| 1.1 | 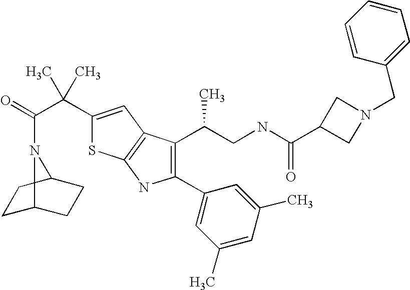 | 623 |
| 1.2 | 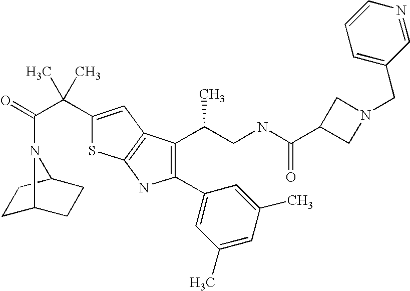 | 624 |
| 1.3 | 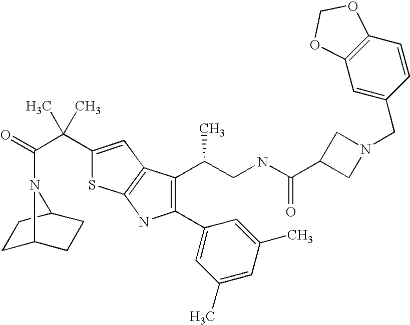 | 667 |
| 1.4 | 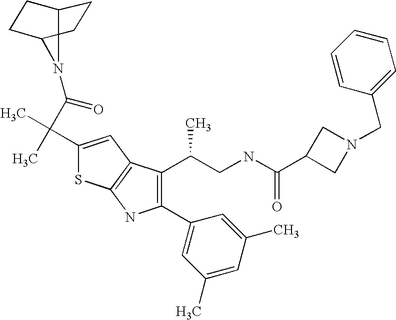 | 639 |
| 1.5 | 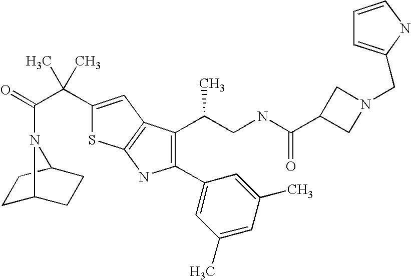 | 612 |
| 1.6 | 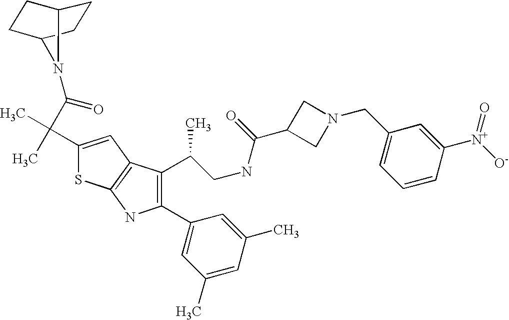 | 668 |
| 1.7 | 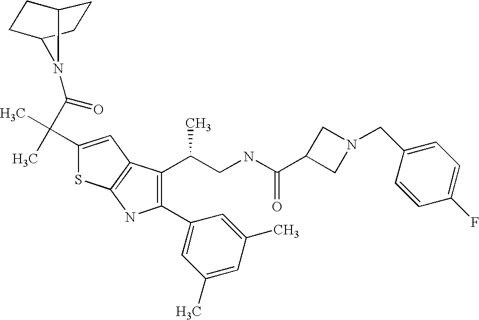 | 641 |
| 1.8 | 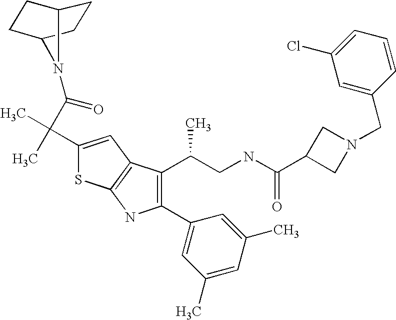 | 657 |
| 1.9 | 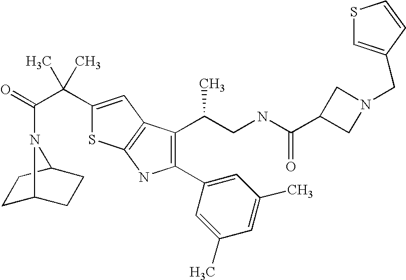 | 629 |
| 1.10 | 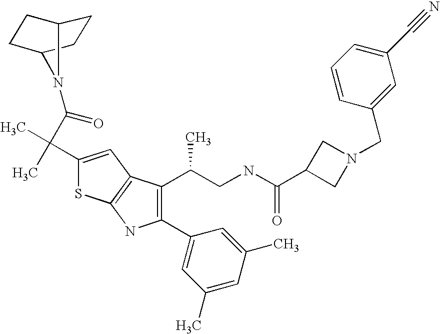 | 648 |
| 1.11 | 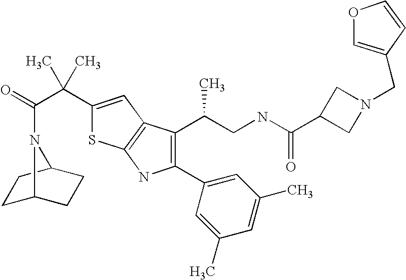 | 613 |
| 1.12 | 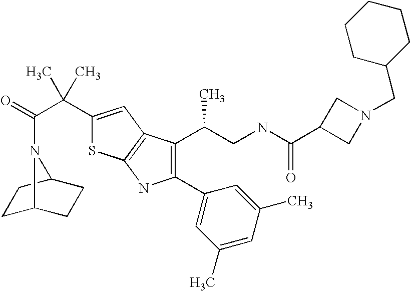 | 629 |
| 1.13 | 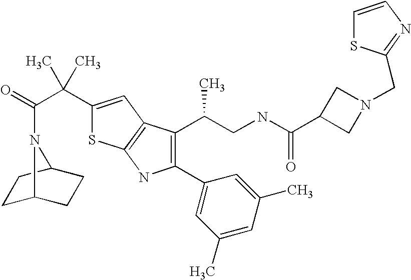 | 630 |

### Example 2

### 2-(1,1-dimethyl-2-oxo-2-azabicyclo[2.2.1]heptan-7-ylethyl)-4-[1-methoxy-2-(4-{pyrrolidin-1-ylcarbonylmethyl}-piperazin-1-yl)ethyl]-5-(3,5-dimethylphenyl)-6H-thieno[2,3-b]pyrrole

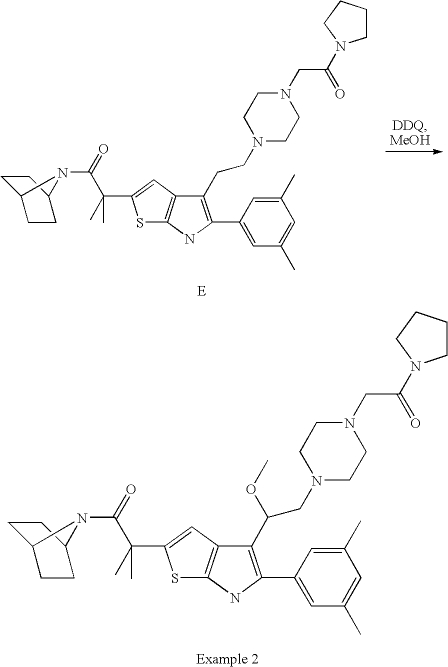

To a solution of E (247 mg ; 0.40 mmol) in methanol (4 ml) was added at room temperature DDQ (0.90 mmol, 2.25 equiv.). The reaction mixture was stirred overnight at 40° C. After removal of solvents under reduced pressure, a saturated aqueous solution of NaHCO₃ was added and extracted twice with dichloromethane. The solvent was evaporated and the residue purified by flash chromatography eluting with increasingly polar mixtures of NH₃ 3.5 M in MeOH/CH₂Cl₂ (2-6% NH₃—MeOH) to give Example 2 as an orange foam (147 mg).

Yield: 57% ¹H NMR spectrum (CDCl₃): 1.20-1.35 (m, 4H); 1.40-1.80 (m, 4H); 1.63 (s, 6H); 1.83 (m, 2H); 1.94 (m, 2H); 2.35 (s, 6H); 2.5-2.7 (m, 10H); 3.06 (s, 3H); 3.11 (m, 2H); 3.47 (m, 4H); 4.10-4.20 (s br, 1H); 4.65-4.75 (s br, 2H); 6.90 (s, 1H); 6.97 (s, 1H); 7.07 (s, 2H); 8.30 (s, 1H). MS-ESI: 646 [M+H]⁺

The starting material E was prepared as follows:

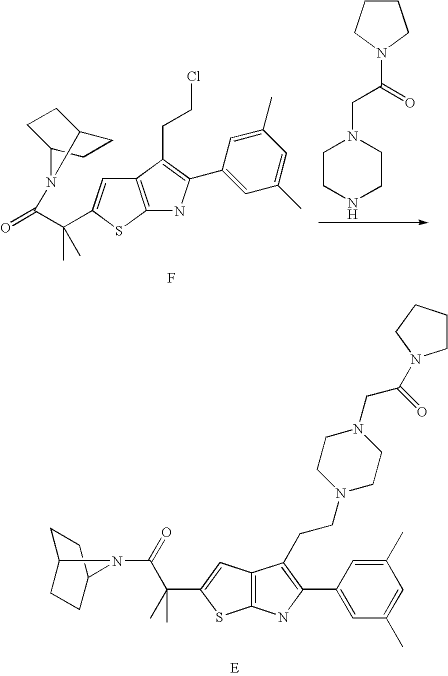

A mixture of F (0.182 g; 0.4 mmol), N-(pyrrolidinocarbonylmethyl) piperazine (0.095 g, 0.48 mmol), NaI (0.072 g; 0.48 mmol) and K₂CO₃ (0.067 g; 0.48 mmol) in acetonitrile (4 ml) was heated at 80° C. under argon atmosphere for 8 hours. The crude mixture was evaporated and purified by flash chromatography eluting with a gradient 2-5% of 3.5 N NH₃ in MeOH/methylene chloride to give after trituration in ether/pentane E as a solid.

Yield: 42% ¹H NMR (CDCl₃): 1.15-1.4 (m, 6H); 1.45-1.75 (m, 6H); 1.59 (s, 6H); 1.8-2 (m, 4H); 2.32 (s, 6H); 2.45-2.75 (m, 6H); 2.9-3 (m, 2H); 3.1 (s, 2H); 3.44-3.5 (m, 4H); 4-4.2 (m, br, 1H); 4.6-4.8 (m, br, 1H); 6.72 (s, 1H); 6.92 (s, 1H); 7.04 (s, 2H); 8.13 (s, 1H). MS-ESI: 616 [M+H]⁺

The intermediate F was prepared as follows:

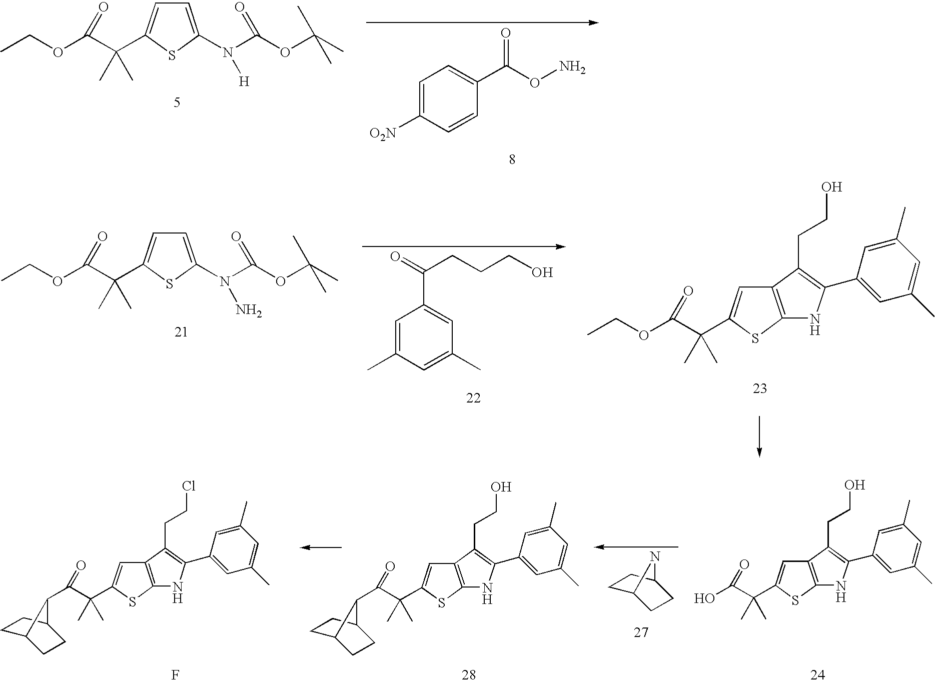

To a suspension of sodium hydride (44.6 g; 1.12 mol) in DMF (700 ml) at 10° C., was added a solution of 5 (290 g; 930 mmol) in DMF (1 l) over a period of 5 minutes. The resulting orange suspension was allowed to warm to room temperature and stirred for 2 hours. The resulting solution was cooled to −5° C. in an acetone/ice bath and a solution of 8 (201 g; 1.02 mol) in DMF (1.4 l ) was added over a period of 1 hour. During this period additional DMF (1 l) was added to mobilize the thick precipitate which formed. The resulting suspension was allowed to warm to room temperature and stirred over night after which HPLC showed no remaining starting material. The suspension was poured into water (6 l) and extracted with diethyl ether (3×2 l). The organic extracts were combined and concentrated to approximately 3 l and washed with water (4×1.5 l ), a saturated solution of brine (1 l), dried over magnesium sulfate and evaporated to dryness to afford the free base as an off-white solid in quantitative yield. To a stirred solution of the free base (150 g; 457 mmol) in diethyl ether (1.2 l) and heptane (600 ml) at 0° C., was added a 4.0M solution of HCl in 1,4-dioxane (145 ml; 570 mmol) over a period of 1 hour. The resulting thick, white precipitate was collected by filtration, washed with a mixture of diethyl ether-heptane (1:1, 500 ml) and dried to a constant weight to afford the 21.HCl (160.3 g) as a white solid.

Yield: 96% MS-ESI: 328 [M+H]⁺

To a stirred solution of 21 (141 g; 380 mmol) in 2-butanol (1.3 l) was added 22 (104 g; 540 mmol) and zinc chloride (106 g; 770 mmol). The resulting suspension was heated at 100° C. for 8 hours after which HPLC showed no remaining starting material. The resulting dark brown solution was evaporated to dryness on a rotary evaporator. The resulting dark brown residue was dissolved in DCM (100 ml), filtered and the filtrate was purified by flash chromatography eluting with DCM, ethyl acetate (9:1) to afford 23 (98 g) as a brown solid.

Yield: 67% MS-ESI: 386 [M+H]⁺

To a stirred solution of 23 (98 g; 254 mmol) in ethanol (1.8 l) was added 1N NaOH (1.27 l, 1270 mmol). The resulting solution was heated at 60° C. for 4 hours after which HPLC showed no remaining starting material. The reaction mixture was cooled to room temperature and the ethanol was removed on a rotary evaporator. The resulting brown solution was cooled to 5° C. and concentrated HCl was added dropwise with rapid agitation decreasing the pH to 1. The resulting precipitate was collected by filtration, washed to a neutral pH with water (3×1 l) and dried to a constant weight in a vacuum oven at 50° C. to afford 24 as a beige solid (68.3 g).

Yield: 75% MS-ESI: 358 [M+H]⁺

To a stirred solution of 24 (35.7 g; 100 mmol) and 27 (57 g; 150 mmol) in DCM (1 l) at 0° C., was added DIPEA (70 ml; 400 mmol) and solid HATU (57 g; 150 mmol) over a period of 15 minutes. The reaction mixture was allowed to warm to room temperature and stirred for 2 hours after which HPLC showed no remaining starting material. The reaction mixture was washed with a saturated aqueous solution of citric acid (350 ml), a saturated solution of sodium bicarbonate (350 ml) and water (3×350 ml). The organic layer was dried over anhydrous magnesium sulfate, filtered, and evaporated to dryness on a rotary evaporator. The resulting oily residue was triturated with ethyl acetate (100 ml) and the resulting precipitate collected by filtration and dried to a constant weight in a vacuum oven at 40° C. to afford 28 (31.4 g) as a beige solid.

Yield: 69% MS-ESI: 437 [M+H]⁺

To a stirred solution of 28 (29.7 g; 68.1 mmol) in DCM (700 ml) at 0° C. was added dropwise neat thionyl chloride (6 ml; 81.7 mmol) The mixture was allowed to warm to room temperature and stirred for a period of 2 h after which HPLC showed no remaining starting material. The reaction mixture was evaporated and purified by flash chromatography, eluting with methylene chloride, AcOEt (9:1) to give 29 as beige foam. The foam was triturated with diethyl ether (100 ml) and the resulting solid collected by filtration, washed with diethyl ether (2×50 ml) and dried to a constant weight in a vacuum oven at 40° C. to afford 29 as a white solid (26.5 g).

Yield: 85% MS-ESI: 454 [M+H]^{+ 1}H NMR (DMSO-d₆), 1.19-1.41 (m, 4H); 1.45-1.59 (m, 10H); 2.32 (s, 6H); 3.14 (t, 2H); 3.83 (t, 4H); 4.13 (br s, 1H); 4.43 (br s, 1H); 6.89-6.93 (two overlapping s, 2H); 7.08 (s, 2H).

The intermediate amine 27 was synthesised as follows:

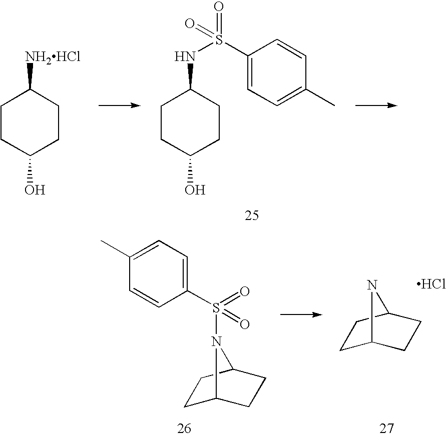

To a stirred suspension of trans-4-aminocyclohexanol (300 g; 1.98 mol) in isopropanol (3.5 l) at 0° C. was added triethylamine (1,1 l; 7.92 mol) followed by solid p-toluenesulfonyl chloride (377 g; 1.98 mmol) over a period of 30 minutes. The reaction mixture was heated at 60° C. for 2 hours after which HPLC showed no remaining starting material. The resulting suspension was cooled to room temperature and the precipitate of triethylamine hydrochloride removed by filtration. The filtrate was evaporated to dryness on a rotary evaporator to afford a colourless oil which was dissolved in ethyl acetate (3 l ), washed with 0.5N HCl (800 ml), water (1.5 l) and dried over MgSO₄. The solvent was evaporated on a rotary evaporator to afford 25 (456.5 g) as a white crystalline solid.

Yield: 86% MS-ESI: 270 [M+H]⁺

To a stirred solution of 25 (600 g; 2.23 mol) in THF (2 l) at −10° C. in an ice/acetone bath, was added triphenylphosphine (700 g; 2.67 mol) followed by di-tert-butylazadicarboxylate (DTBAD) (564 g; 2.45 mol) in THF (1.5 l) over a period of 1.5 hours maintaining the internal temperature below 10° C. The ice/acetone bath was removed and reaction mixture was allowed to warm to room temperature over a period of 1.5 hours after which HPLC showed no remaining starting material. The reaction mixture was evaporated to dryness and the residue was crystallised from hot MeOH (2.8 l). The resulting crystalline suspension was cooled to 0° C. and the crystals collected by filtration, washed with cold MeOH (2×200 ml) and dried to a constant weight in a vacuum oven to afford 26 (378.2 g) as a white crystalline solid routinely contaminated with approximately 10% (w/w) of triphenylphosphine oxide

Yield: 68% MS-ESI: 252 [M+H]⁺

In two separate batches: To a stirred solution of 26 (380 g; 1.51 mol) in THF (3 l) at 0° C. was added solid pellets of lithium aluminium hydride (229.4 g; 6.04 mol) over a period of 2 hours under a blanket of nitrogen. The resulting grey suspension was allowed to warm to room temperature and stirred for 4 days after which HPLC showed no remaining starting material. The reaction mixture was diluted with THF (1 l), cooled to 0° C. and solid sodium sulfate decahydrate was added over a period of 2 hours with rapid agitation. When the effervescence had subr sided, the resulting suspension was filtered and the filtrate acidified with gaseous HCl affording a thick white precipitate which was collected by filtration, washed with THF (2×500 ml) and dried to a constant weight to afford 108 (batch 1: 86.8 g; 43%) (batch 2: 97.3 g; 49%) as a white solid. The filter cakes obtained from the first filtration were suspended in 6N NaOH (400 ml) and filtered. The filtrate was extracted with diethyl ether (4 l). The organic layer was acidified with gaseous HCl affording a thick white precipitate which was collected by filtration, washed with diethyl ether (2×500 ml) and dried to a constant weight in a vacuum oven at 40° C. to afford 27.HCl (105.9 g) as a white solid.

Yield: 72% ¹H NMR (DMSO-d₆), 1.57 (m, 4H); 1.86 (m, 4H); 4.12 (s, 2H); 8.80-9.05 (br s, 1H).

Using analogous methods, the following compounds were also prepared:
2-(1,1-dimethyl-2-oxo-2-azabicyclo[2.2.1]heptan-7-ylethyl)-4-[1-methoxy-2-(4-{morpholinocarbonyl}-piperazin-1-yl)ethyl]-5-(3,5-dimethylphenyl)-6H-thieno[2,3-b]pyrrole; 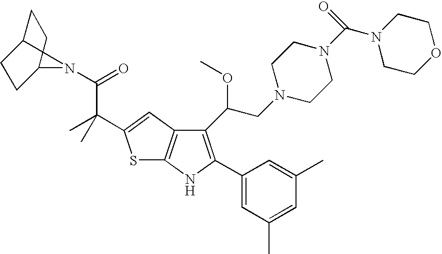
2-(1,1-dimethyl-2-oxo-2-azabicyclo[2.2.1]heptan-7-ylethyl)-4-[1-methoxy-2-(4-{4-methoxypiperidin-1-ylcarbonyl}-piperazin-1-yl)ethyl]-5-(3,5-dimethylphenyl)-6H-thieno[2,3-b]pyrrole

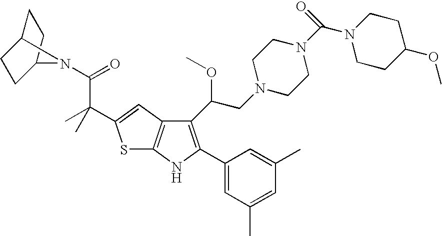

### Example 3

### 2-(1,1-dimethyl-2-oxo-2-azabicyclo[2.2.1]heptan-7-ylethyl)-4-[2-(4-{1-carbamoylbutlylaminocarbonyl}-piperidin-1-yl)ethyl]-5-(3,5-dimethylphenyl)-6H-thieno[2,3-b]pyrrole

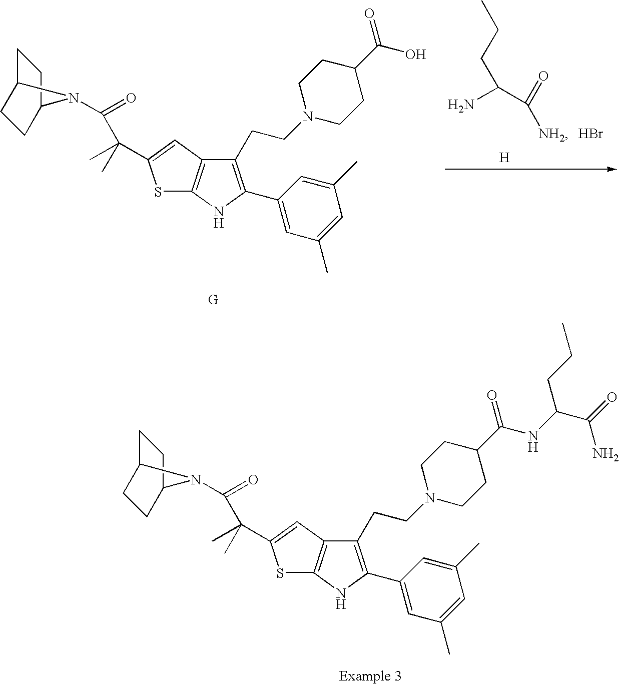

To a solution of acid G (0.164 g, 0.3 mmole) in DMF (1.5 ml) were added HATU (0.183 g, 0.48 mmole), and diisopropylamine (0.157 ml, 0.116 g, 0.9 mmole). After stirring for 1 hour at ambient temperature for 1 hour, amine H (0.074 g; 0.9 mmol) was added. After stirring overnight, the mixture was purified by reverse phase chromatography, eluting with a gradient 10-90% acetonitrile in H₂O/(NH₄)₂CO₃ 2 g/l to give after evaporation and trituration in ether/pentane Example 3 as a solid.

Yield: 50% MS-ESI: 646 (M+H⁺). ¹H NMR (DMSOd₆): 0.84 (t, 3H), 1.15-1.35 (m, 6H), 1.35-1.75 (m, 16H), 1.85-2.00 (m, 2H), 2.15-2.25 (m, 1H), 2.31 (s, 6H); 2.8-3.05 (m, 4H), 4.00-4.60 (m, 3H), 6.8 (s, 1H), 6.91 (s, 2H), 6.91 (d, 2H), 7.09(s, 2H), 7.27 (s, 1H), 7.75 (m, 1H).

The starting material G was prepared according to the following scheme.

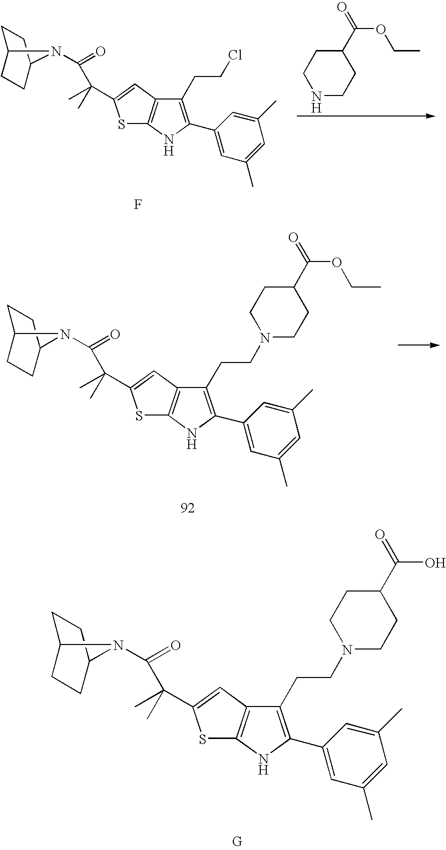

A mixture of 29 (4.55 g; 0.01 mol), 4-ethoxycarbonyl piperidine (2.36 g; 0.015 mol), triethylamine (1.53 ml; 0.011 mol) and NaI (1.5 g; 0.01 mol) in DMA (45 ml) was heated at 110° C. under argon atmosphere for 4 hours. After extraction with ethyl acetate and evaporation, the mixture was purified by flash chromatography, eluting with a gradient 80-100% ethyl acetate/petroleum ether to give 92.

Yield: 62% ¹H NMR (CDCl₃): 1.25 (t, 3H); 1.2-1.45 (m, 4H); 1.5-1.8 (m, 4H); 1.62 (s, 6H); 1.7-2 (m, 4H); 2.05-2.15 (m, 2H); 2.25-2.35 (m, 1H); 2.35 (s, 6H); 2.64-2.67 (m, 2H); 2.93-2.98 (m, 4H); 4.13 (q, 2H); 4.0-4.2 (br m, 1H); 4.6-4.8 (br m, 1H); 6.74 (s, 1H); 6.94 (s, 1H); 7.07 (s, 2H); 8.13 (s, 1H).

A solution of 92 (3.61 g; 0.627 mmol) in 2N NaOH (5 ml) and EtOH (100 ml) was heated at 60)C. for 2 hours. After extraction with methylene chloride/methanol (50/50)and evaporation, the residue was triturated in ether to give G as a solid.

Yield: 93% ¹H NMR (DMSOd₆, AcOH): 1.30 (m, 4H); 1.40-1.70 (m, 4H); 1.53 (s, 6H); 1.80-2.00 (m, 4H); 2.05 (m, 2H); 2.34 (s, 6H); 2.65 (m, br, 1H); 3.14 (m, 2H); 3.27 (m, 2H); 3.30-3.60 (m, 2H); 4.10 (m, br, 1H); 4.50 (m, br, 1H); 6.96 (m, 2H); 7.09 (m, 2H).

Following a procedure similar to that described in Example 3, the following compounds compounds were prepared.

| Example | | MS-ESI |
|---|---|---|
| 3.1 | 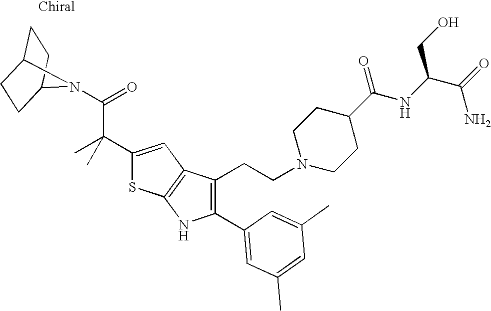 | 634 |
| 3.2 | 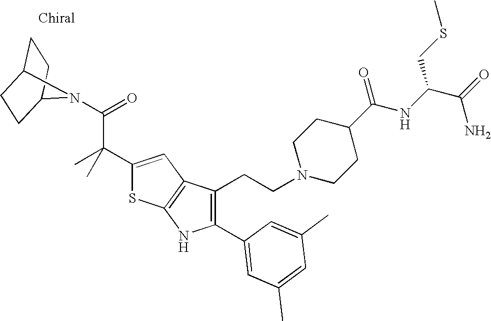 | 665 |
| 3.3 | 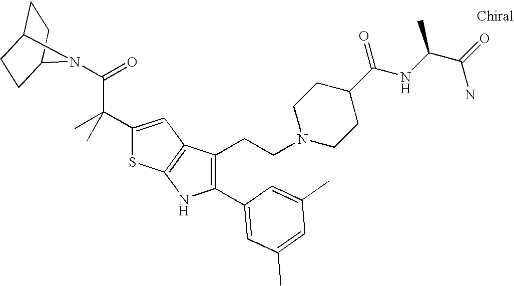 | 618 |
| 3.4 | 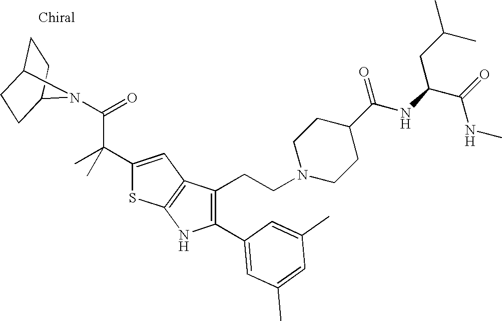 | 675 |
| 3.5 | 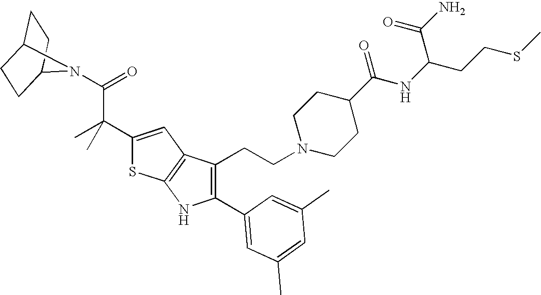 | 679 |
| 3.6 | 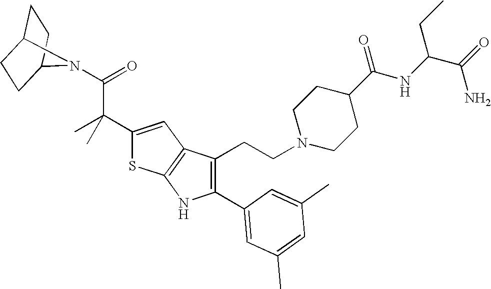 | 632 |
| 3.7 | 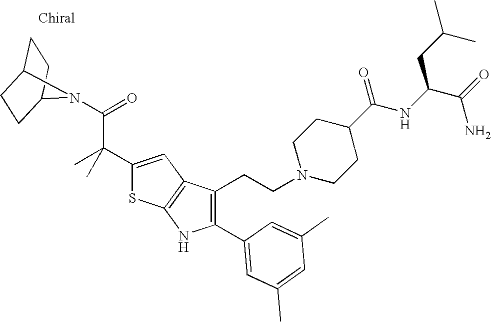 | 660 |
| 3.9 | 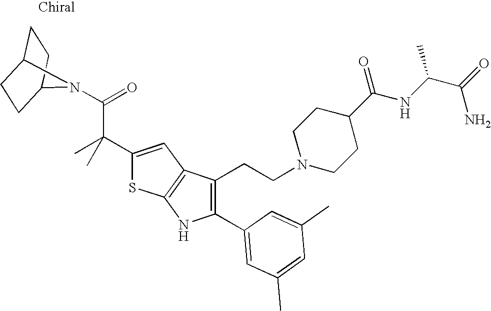 | 618 |
| 3.10 | 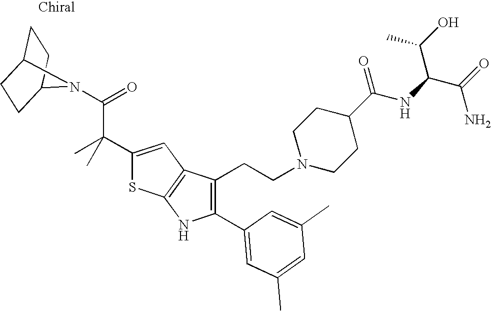 | 648 |
| 3.11 | 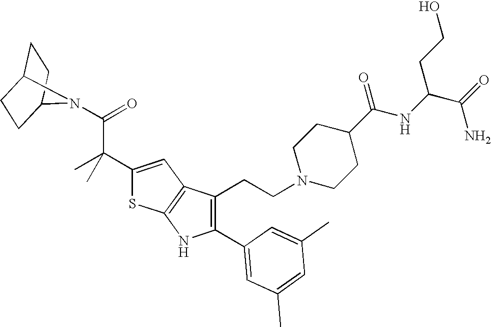 | 648 |
| 3.12 | 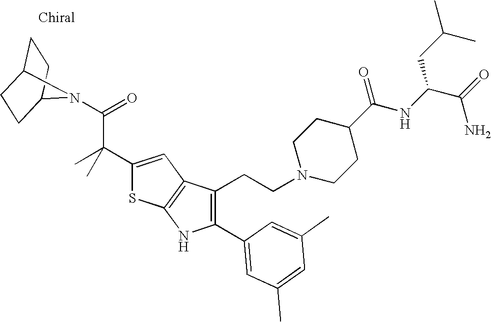 | 660 |

### Example 4

### 2-(1,1-dimethyl-2-oxo-2-azabicyclo[2.2.1]heptan-7-ylethyl)-4-[2-(4-{morpholinocarbonyl-1,1-dimethylmethylene}-piperidin-1-yl)ethyl]-5-(3,5-dimethylphenyl)-6H-thieno[2,3-b]pyrrole

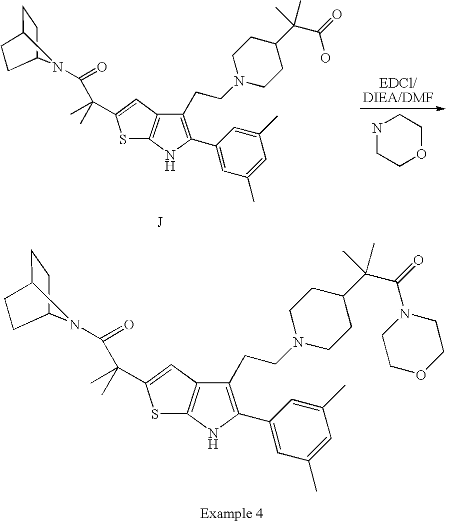

To a solution of J (0.196 g; 0.33 mmol) in DMF (2 ml) was added HATU (0.202 g; 0.53 mml) and DIEA (0.117 ml; 0.67 mmol). The mixture was stirred at ambient temperature for 1 hour; morpholine (0.059 ml; 0.67 mmol) was added After further stirring for 2 hours, the mixture was purified by reverse phase chromatography, eluting with a gradient 10-90% acetonitrile in H₂O/(NH₄)₂CO₃ 2 g/l to give after evaporation and trituration in ether/pentane Example 4 as a solid.

Yield: 20% ¹H NMR (CDCl₃+acetic acid): 1.21 (s, 6H), 1.2-1.45 (m, 4H ), 1.45-1.65 (m, 2H), 1.64 (s, 6H), 1.60-1.85 (m, 6H), 2.00-2.10 (m, 1H), 2.36 (s, 6H), 2.65-2.85 (m, 2H), 3.22 (s, 4H), 3.6-3.80 (m, 10H), 4.2 (s, 1H), 4.75 (s, 1H), 6.85 (s, 1H), 6.97 (s, 1H), 7.02 (s, 2H).

The starting material was prepared as follows:

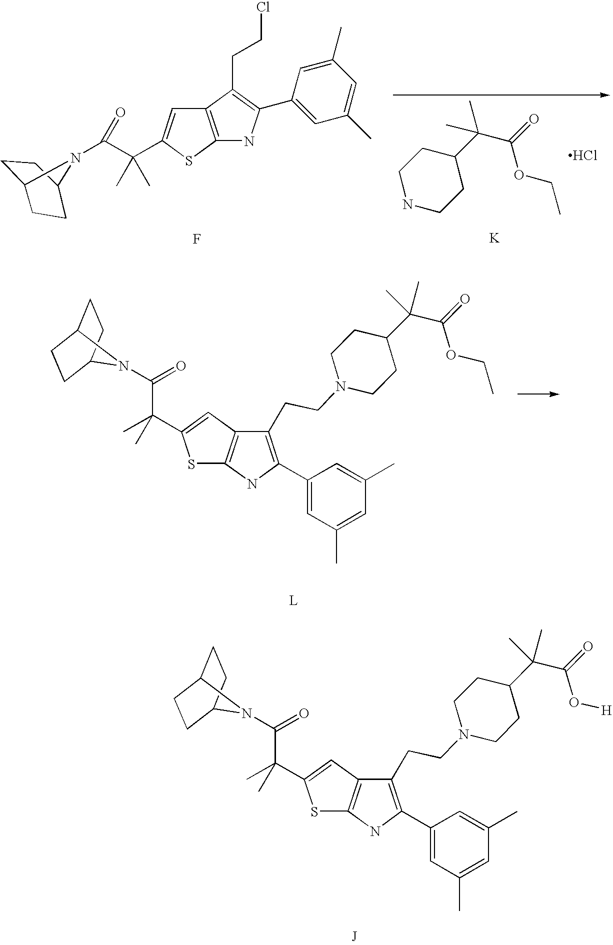

A mixture of F (see Example 2) (1.93 g, 4.25 mmol); K (1 g; 4.25 mmol) and K₂CO₃ in acetonitrile (12 ml) and DMF (5 ml) was heated at 90° C. for 4 hours. After extraction with ethyl acetate, the organic layer was evaporated to dryness to give L as a foam.

Yield: 82% ¹H NMR (CDCl₃): 1.12 (s, 3H), 1.27 (t, 3H), (1.25-1.8, 13H), 1.62 (s, 6H), 2.00 (m, 2H), 2.6-2.7 (m, 2H), 2.9-3.0 (m, 2H), 4.0-4.2 (m, 1H), 4.13 (q, 2H), 4.60-4.85 (m, 1H), 6.74 (s, 1H), 6.94 (s, 1H ), 7.06 (s, 2H ), 8.14 (s, 1NH).

A solution of L (1.19 g; 1.93 mmol) in ethanol (10 ml) was treated with NaOH 2N (2.7 ml; 2.5.4 mmol) at 80° C. overnight. After evaporation to dryness, the residue was taken up with EtAc/H2O; the aqueous layer was acidified and extracted with methylene chloride to give J as a solid.

Yield: 39% ¹H NMR (CDCl3): 1.19 (d, 6H), 1.32 (s, 6H), 1.2-2.3 (m, 11H), 2.4 (s, 6H ), 2.50-2.65 (m, 2H), 3.00-3.65 (m, 8H), 4.00-4.30 (m, 1H), 4.50-4.85 (m, 1H), 6.86 (s, 1H), 6.97 (s, 1H), 7.04 (s, 1H), 7.10 (s, 1H), 8.32 (1, NH)

### Example 5

### 2-(1,1-dimethyl-2-oxo-2-azabicyclo[2.2.1]heptan-7-ylethyl)-4-[2-(4-{3-hydroxypyrrolidin-1-ylcarbonyl-1,1-dimethylmethylene}-piperidin-1-yl)ethyl]-5-(3,5-dimethylphenyl)-6H-thieno[2,3-b]pyrrole

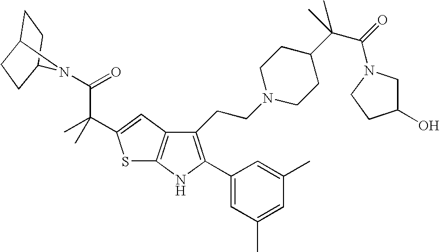

Example 5 was prepared using a procedure similar to that described for Example 4

Yield: 16% MS-ESI: 646 (M+H⁺). ¹H NMR (CDCl₃+acetic acid): 1.21 (s, 6H), 1.2-2.00 (m, 14H), 1.63 (s, 6H), 2.00-2.20 (s, 1H), 2.36 (s, 6H), 2.7-2.95 (m, 2H), 3.22 (s, 4H), 3.5-3.9 (m, 6H), 4.2 (s, 1H), 4.5 (s, 1H), 4.75 (s, 1H), 6.85 (s, 1H), 6.98 (s, 1H), 7.01 (s, 2H).

Therapeutic Uses

Compounds of Formula (I) are provided as medicaments for antagonising gonadotropin releasing hormone (GnRH) activity in a patient, eg, in men and/or women. To this end, a compound of Formula (I) can be provided as part of a pharmaceutical formulation which also includes a pharmaceutically acceptable diluent or carrier (eg, water). The formulation may be in the form of tablets, capsules, granules, powders, syrups, emulsions (eg, lipid emulsions), suppositories, ointments, creams, drops, suspensions (eg, aqueous or oily suspensions) or solutions (eg, aqueous or oily solutions). If desired, the formulation may include one or more additional substances independently selected from stabilising agents, wetting agents, emulsifying agents, buffers, lactose, sialic acid, magnesium stearate, terra alba, sucrose, corn starch, talc, gelatin, agar, pectin, peanut oil, olive oil, cacao butter and ethylene glycol.

The compound is preferably orally administered to a patient, but other routes of administration are possible, such as parenteral or rectal administration. For intravenous, subcutaneous or intramuscular administration, the patient may receive a daily dose of 0.1 mgkg⁻¹ to 30 mgkg⁻¹ (preferably, 5 mgkg⁻¹ to 20 mgkg⁻¹) of the compound, the compound being administered 1 to 4 times per day. The intravenous, subcutaneous and intramuscular dose may be given by means of a bolus injection. Alternatively, the intravenous dose may be given by continuous infusion over a period of time. Alternatively, the patient may receive a daily oral dose which is approximately equivalent to the daily parenteral dose, the composition being administered 1 to 4 times per day. A suitable pharmaceutical formulation is one suitable for oral administration in unit dosage form, for example as a tablet or capsule, which contains between 10 mg and 1 g (preferably, 100 mg and 1 g) of the compound of the invention.

Buffers, pharmaceutically acceptable co-solvents (eg, polyethylene glycol, propylene glycol, glycerol or EtOH) or complexing agents such as hydroxy-propyl β cyclodextrin may be used to aid formulation.

One aspect of the invention relates to the use of compounds according to the invention for reducing the secretion of LH and/or FSH by the pituitary gland of a patient. In this respect, the reduction may be by way of a reduction in biosynthesis of the LH and FSH and/or a reduction in the release of LH and FSH by the pituitary gland. Thus, compounds according to the invention can be used for therapeutically treating and/or preventing a sex hormone related condition in the patient. By “preventing” we mean reducing the patient's risk of contracting the condition. By “treating” we mean eradicating the condition or reducing its severity in the patient. Examples of sex hormone related conditions are: a sex hormone dependent cancer, benign prostatic hypertrophy, myoma of the uterus, endometriosis, polycystic ovarian disease, uterine fibroids, prostatauxe, myoma uteri, hirsutism and precocious puberty. Examples of sex hormone dependent cancers are: prostatic cancer, uterine cancer, breast cancer and pituitary gonadotrophe adenoma.

The compounds of the invention may be used in combination with other drugs and therapies used to treat/prevent sex-hormone related conditions.

If formulated as a fixed dose such combination products employ the compounds of this invention within the dosage range described herein and the other pharmaceutically-active agent within its approved dosage range. Sequential use is contemplated when a combination formulation is inappropriate.

In the field of medical oncology examples of such combinations include combinations with the following categories of therapeutic agent:
i) anti-angiogenic agents (for example linomide, inhibitors of integrin ανβ3 function, angiostatin, endostatin, razoxin, thalidomide) and including vascular endothelial growth factor (VEGF) receptor tyrosine kinase inhibitors (RTKIs) (for example those described in international patent applications publication nos. WO-97/22596, WO-97/30035, WO-97/32856 and WO-98/13354, the entire disclosure of which documents is incorporated herein by reference);
ii) cytostatic agents such as anti-oestrogens (for example tamoxifen, toremifene, raloxifene, droloxifene, iodoxyfene), progestogens (for example megestrol acetate), aromatase inhibitors (for example anastrozole, letrozole, vorazole, exemestane), anti-progestogens, anti-androgens (for example flutamide, nilutamide, bicalutamide, cyproterone acetate), inhibitors of testosterone 5α-dihydroreductase (for example finasteride), anti-invasion agents (for example metalloproteinase inhibitors like marimastat and inhibitors of urokinase plasminogen activator receptor function) and inhibitors of growth factor function, (such growth factors include for example epidermal growth factor (EGF), platelet derived growth factor and hepatocyte growth factor such inhibitors include growth factor antibodies, growth factor receptor antibodies, tyrosine kinase inhibitors and serine/threonine kinase inhibitors),
iii) biological response modifiers (for example interferon);
iv) antibodies (for example edrecolomab); and
v) anti-proliferative/anti-neoplastic drugs and combinations thereof, as used in medical oncology, such as anti-metabolites (for example anti-folates like methotrexate, fluoropyrimidines like 5-fluorouracil, purine and adenosine analogues, cytosine arabinoside); anti-tumour antibiotics (for example anthracyclines like doxorubicin, daunomycin, epirubicin and idarubicin, mitomycin-C, dactinomycin, mithramycin); platinum derivatives (for example cisplatin, carboplatin); alkylating agents (for example nitrogen mustard, melphalan, chlorambucil, busulphan, cyclophosphamide, ifosfamide, nitrosoureas, thiotepa); anti-mitotic agents (for example vinca alkaloids like vincristine and toxoids like taxol, taxotere); enzymes (for example asparaginase); thymidylate synthase inhibitors (for example raltitrexed); topoisomerase inhibitors (for example epipodophyllotoxins like etoposide and teniposide, amsacrine, topotecan, irinotecan).

The compounds of the invention may also be used in combination with surgery or radiotherapy.

Assays

The ability of compounds according to the invention to act as antagonists of GnRH can be determined using the following in vitro assays.

Binding Assay Using Rat Pituitary GnRH Receptor

The assay is performed as follows:
1. Incubate crude plasma membranes prepared from rat pituitary tissues in a Tris.HCl buffer (pH. 7.5, 50 mM) containing bovine serum albumin (0.1%), [I-125]D-t-Bu-Ser6-Pro9-ethyl amide-GnRH, and the test compound. Incubation is at 4° C. for 90 minutes to 2 hours.
2. Rapidly filter and repeatedly wash through a glass fibre filter.
3. Determine the radioactivity of membrane bound radio-ligands using a gamma counter.

From this data, the IC₅₀ of the test compound can be determined as the concentration of the compound required to inhibit radio-ligand binding to GnRH receptors by 50%. Compounds according to the present invention have activity at a concentration from 1 nM to 5 μM.

Binding Assay Using Human GnRH Receptor

Crude membranes prepared from CHO cells expressing human GnRH receptors are sources for the GnRH receptor. The binding activity of compounds according to the invention can be determined as an IC₅₀ which is the compound concentration required to inhibit the specific binding of [¹²⁵I]buserelin to GnRH receptors by 50%. [¹²⁵I]Buserelin (a peptide GnRH analogue) is used here as a radiolabelled ligand of the receptor.

Assay to Determine Inhibition of LH Release

The LH release assay can be used to demonstrate antagonist activity of compounds, as demonstrated by a reduction in GnRH-induced LH release.

Preparation of Pituitary Glands

Pituitary glands obtained from rats are prepared as follows. Suitable rats are Wistar male rats (150-200 g) which have been maintained at a constant temperature (eg,25° C.) on a 12 hour light/12 hour dark cycle. The rats are sacrificed by decapitation before the pituitary glands are aseptically removed to tube containing Hank's Balanced Salt Solution (HBSS). The glands are further processed by:
1. Centrifugation at 250×g for 5 minutes;
2. Aspiration of the HBSS solution;
3. Transfer of the glands to a petri dish before mincing with a scalpel;
4. Transfer of the minced tissue to a centrifuge tube by suspending the tissue three successive times in 10 ml aliquots of HBSS containing 0.2% collagenase and 0.2% hyaluronidase;
5. Cell dispersion by gentle stirring of the tissue suspension while the tube is kept in a water bath at 37° C.;
6. Aspiration 20 to 30 times using a pipette, undigested pituitary fragments being allowed to settle for 3 to 5 minutes;
7. Aspiration of the suspended cells followed by centrifugation at 1200×g for 5 minutes;
8. Re-suspension of the cells in culture medium of DMEM containing 0.37% NaHCO₃, 10% horse serum, 2.5% foetal bovine serum, 1% non essential amino acids, 1% glutamine and 0.1% gentamycin;
9. Treatment of the undigested pituitary fragments 3 times with 30 ml aliquots of the collagenase and hyaluronidase;
10. Pooling of the cell suspensions and dilution to a concentration of 3×10⁵ cells/ml;
11. Placing of 1.0 ml of this suspension in each of a 24 well tray, with the cells being maintained in a humidified 5% CO₂/95% air atmosphere at 37° C. for 3 to 4 days
   Testing of Compounds

The test compound is dissolved in DMSO to a final concentration of 0.5% in the incubation medium.

1.5 hours prior to the assay, the cells are washed three times with DMEM containing 0.37% NaHCO₃, 10% horse serum, 2.5% foetal bovine serum, 1% non essential amino acids (100×), 1% glutamine (100×), 1% penicillin/streptomycin (10,000 units of each per ml) and 25 mM HEPES at pH 7.4. Immediately prior to the assay, the cells are again washed twice in this medium.

Following this, 1 ml of fresh medium containing the test compound and 2 nM GnRH is added to two wells. For other test compounds (where it is desired to test more than one compound), these are added to other respective duplicate wells. Incubation is then carried out at 37° C. for three hours.

Following incubation, each well is analysed by removing the medium from the well and centrifuging the medium at 2000×g for 15 minutes to remove any cellular material. The supernatant is removed and assayed for LH content using a double antibody radio-immuno assay. Comparison with a suitable control (no test compound) is used to determine whether the test compound reduces LH release. Compounds according to the present invention have activity at a concentration from 1 nM to 5 μM.

1. A compound of Formula (I), 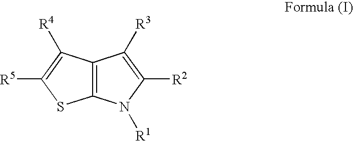
wherein:
R¹ is selected from: hydrogen, optionally substituted C₁₋₆alkyl, optionally substituted aryl or optionally substituted arylC₁₋₆alkyl, wherein the optional substituents are selected from C₁₋₄alkyl, nitro, cyano, fluoro and C₁₋₄alkoxy;
R² is hydrogen, optionally substituted C₁₋₆alkyl or an optionally substituted mono or bi-cyclic aromatic ring, wherein the optional substituents are 1, 2 or 3 substituents independently selected from: cyano, R^{e}R^{f}N—, C₁₋₆alkyl, C₁₋₆alkoxy, halo, haloC₁₋₆alkyl or haloC₁₋₆alkoxy wherein R^{e} and R^{f} are independently selected from hydrogen, C₁₋₆alkyl or aryl;
R³ is selected from a group of Formula (IIa) to Formula (IId): 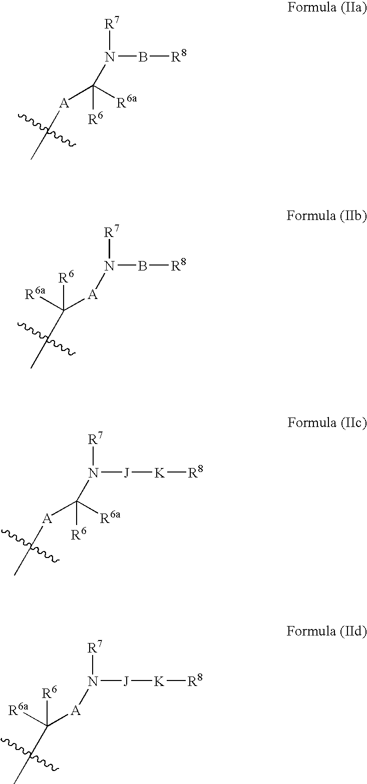
R⁴ is selected from hydrogen, C₁₋₄alkyl or halo;
R⁵ is selected from a group of Formula III-a; III-b; III-c; III-d; III-e; III-f, III-g, III-h, III-i, or III-j, III-k, III-l, III-m, III-n, III-o or III-p 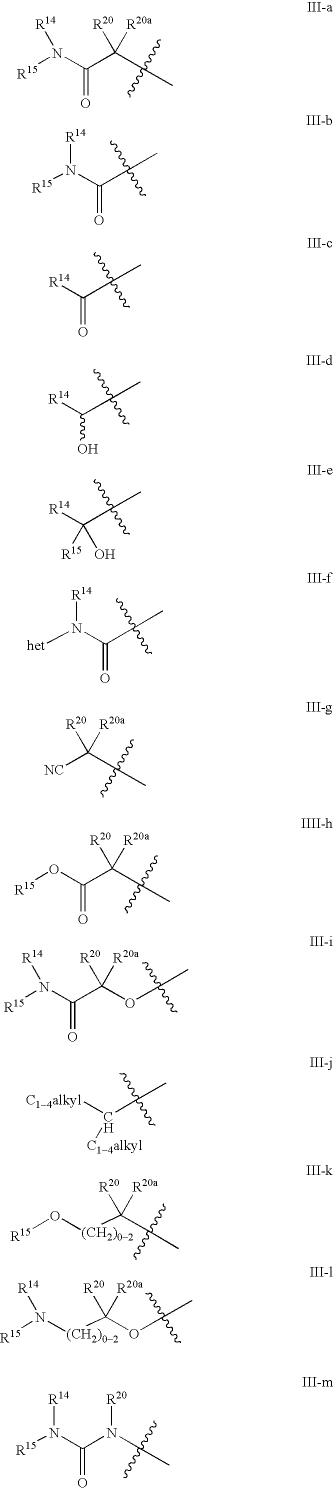 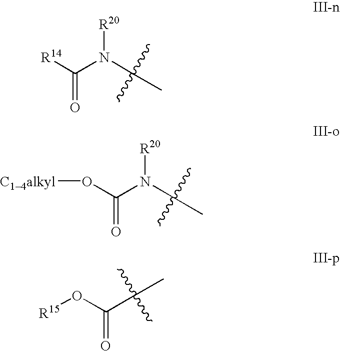
wherein:
het represents an optionally substituted 3- to 8-membered heterocyclic ring containing from 1 to 4 heteroatoms independently selected from O, N and S, wherein the optional substituents are selected from 1-2 groups selected from R¹² and R¹³;
R¹⁴ and R¹⁵ are selected from:
(i) R¹⁴ is selected from hydrogen; optionally substituted C₁₋₈alkyl; optionally substituted aryl; —R^{d}—Ar, where R^{d} represents C₁₋₈alkylene and Ar represents optionally substituted aryl; and optionally substituted 3- to 8-membered heterocyclic ring optionally containing from 1 to 3 further heteroatoms independently selected from I, N and S; and R¹⁵ is selected from hydrogen; optionally substituted C₁₋₈alkyl and optionally substituted aryl;
(ii) when R⁵ represents a group of Formula III-a, III-b, III-i, III-l or III-m, then the group NR¹⁴(—R¹⁵) additionally represents an optionally substituted 3- to 8-membered heterocyclic ring optionally containing from 1 to 3 further heteroatoms independently selected from O, N and S; or
(iii) when R⁵ represents structure III-e, 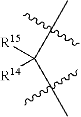
represents an optionally substituted 3- to 8-membered heterocyclic ring optionally containing from 1 to 4 heteroatoms independently selected from O, N and S;
R²⁰ and R^{20a} are independently selected from hydrogen, fluoro or optionally substituted C₁₋₈alkyl, or R²⁰ and R^{20a} together with the carbon atom to which they are attached form an optionally substituted 3 to 7-membered cycloalkyl ring;
R⁶ and R^{6a} are independently selected from hydrogen, fluoro, optionally substituted C₁₋₆alkyl, C₁₋₆alkoxy, N-C₁₋₆alkylamino and N,N-diC₁₋₆alkylamino or R⁶ and R^{6a} taken together and the carbon atom to which they are attached form a carbocyclic ring of 3-7 atoms or R⁶ and R^{6a} taken together and the carbon atom to which they are attached form a carbonyl group;
or when A is not a direct bond, the group 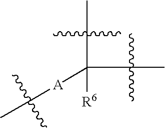
forms a carbocyclic ring of 3-7 carbon atoms or a heterocyclic ring containing one or more heteroatoms;
or the group 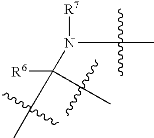
forms a heterocyclic ring containing 3-7 carbon atoms and one or more heteroatoms;
R⁷ is selected from: hydrogen or C₁₋₆alkyl;
R⁸ is selected from:
(i) hydrogen, C₁₋₆alkyl, C₂₋₆alkenyl, C₂₋₆alkynyl, haloC₁₋₆alkyl, C₁₋₄alkoxyC₁₋₄alkyl, hydroxy, hydroxyC₁₋₆alkyl, cyano, N-C₁₋₄alkylamino, N,N-di-C₁₋₄alkylamino, C₁₋₆alkyl-S(Oₙ)—, —O—R^{b}, —NR^{b}R^{c}, —C(O)—R^{b}, —C(O)O—R^{b}, —CONR^{b}R^{c}, NH—C(O)—R^{b} or —S(Oₙ)NR^{b}R^{c},
where R^{b} and R^{c} are independently selected from hydrogen and C₁₋₆alkyl (e.g. C₁₋₄alkyl) optionally substituted with hydroxy, amino, N-C₁₋₄alkylamino, N,N-di-C₁₋₄alkylamino, HO—C₂₋₄alkyl-NH— or HO—C₂₋₄alkyl-N(C₁₋₄alkyl)-;
(ii) nitro when B is a group of Formula (IV) and X is CH and p is 0;
(iii) carbocyclyl (such as C₃₋₇cycloalkyl or aryl) or arylC₁₋₆alkyl each of which is optionally substituted by R¹², or R¹³;
(iv) heterocyclyl or heterocyclylC₁₋₆alkyl each of which is optionally substituted by up to 4 substituents independently selected from R¹² or R¹³ and where any nitrogen atoms within a heterocyclyl group are, where chemically allowed, optionally in their oxidised (N→O, N—OH) state;
R¹² is independently selected from: halo, hydroxy, hydroxyC₁₋₆alkyl, oxo, cyano, cyanoC₁₋₆alkyl, nitro, carboxyl, C₁₋₆alkyl, C₁₋₆alkoxy, C₁₋₆alkoxyC₁₋₄alkyl, C₁₋₆alkoxycarbonylC₀₋₄alkyl, C₁₋₆alkanoylC₀₋₄alkyl, C₁₋₆alkanoyloxyC₀₋₄alkyl, C₂₋₆alkenyl, C₁₋₃perfluoroalkyl-, C₁₋₃perfluoroalkoxy, aryl, arylC₁₋₆alkyl, heterocyclyl, heterocyclylC₁₋₆alkyl, aminoC₀₋₄alkyl, N-C₁₋₄alkylaminoC₀₋₄alkyl, N,N-di-C₁₋₄alkylaminoC₀₋₄alkyl, carbamoyl, N-C₁₋₄alkylcarbamoylC₀₋₂alkyl, N,N-di-C₁₋₄alkylaminocarbamoylC₀₋₂alkyl, aminocarbonylC₀₋₄alkyl, N-C₁₋₆alkyaminocarbonylC₀₋₄alkyl, N,N-C₁₋₆alkyaminocarbonylC₀₋₄alkyl, C₁₋₆alkyl-S(O)ₙ-aminoC₀₋₄alkyl-, aryl-S(O)ₙ-aminoC₀₋₂alkyl-, C₁₋₃perfluoroalkyl-S(O)ₙ-aminoC-₀₋₂alkyl-; C₁₋₆alkylamino-S(O)ₙ-C₀₋₂alkyl-, arylamino-S(O)ₙ-C₀₋₂alkyl-, C₁₋₃perfluoroalkylamino-S(O)ₙ-C₀₋₂alkyl-, C₁₋₆alkanoylamino-S(O)ₙ-C₀₋₂alkyl-; arylcarbonylamino-S(O)ₙ-C₀₋₂alkyl-, C₁₋₆alkyl-S(O)ₙ-C₀₋₂alkyl-, aryl-S(O)ₙ-C₀₋₂alkyl-, C₁₋₃perfluoroalkyl-, C₁₋₃perfluoroalkoxyC₀₋₂alkyl; R^{9′}OC(O)(CH₂)_{w}—, R^{9″}R^{10″}N(CH₂)_{w}—, R^{9′}R^{10′}NC(O)(CH₂)_{w}—, R⁹R¹⁰NC(O)N(R⁹)(CH₂)_{w}—, R⁹OC(O)N(R⁹)(CH₂)_{w}—, or halo, wherein w is an integer between 0 and 4 and R⁹ and R¹⁰ are independently selected from hydrogen, C₁₋₄alkyl, C₁₋₄alkylsulphonyl and C₃₋₇carbocyclyl, R^{9′} and R^{10′} are independently selected from C₁₋₄alkylsulphonyl and C₃₋₇carbocyclyl, and R^{9″} and R^{10″} are C₃₋₇carbocyclyl; wherein an amino or an aryl group within R¹² is optionally substituted by C₁₋₄alkyl;
R¹³ is —C(O)—R¹⁶;
R¹⁶ is selected from an amino acid derivative or an amide of an amino acid derivative;
R¹⁷ is hydrogen or C₁₋₄alkyl;
A is selected from:
(i) a direct bond;
(ii) optionally substituted C₁₋₅alkylene wherein the optional substituents are independently selected from: hydroxy, hydroxyC₁₋₆alkyl, C₁₋₆alkyl, C₁₋₆alkoxy, C₁₋₄alkoxyC₁₋₄alkyl, aryl, arylC₁₋₆alkyl, carbonyl or carbonylmethyl;
(iii) a carbocyclic ring of 3-7 atoms;
(iv) a carbonyl group or —C(O)—C(R^{d}R^{d})—, wherein each R^{d} is independently selected from hydrogen and C₁₋₂alkyl;
or when R³ is a group of Formula (IIa) or (IIb), the group 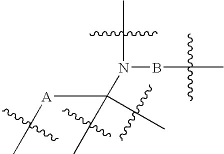
forms a heterocyclic ring containing 3-7 carbon atoms and one or more heteroatoms;
or when R³ is a group of Formula (IIa), (IIb), (IIc) or (IId), the group 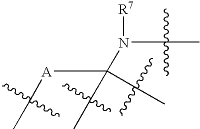
forms a heterocyclic ring containing 3-7 carbon atoms and one or more heteroatoms;
B is selected from:
(i) a direct bond;
(ii) a group of Formula (IV) 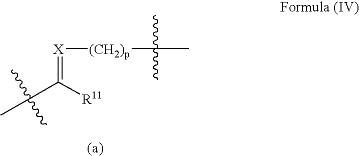
wherein:
X is selected from N or CH,
wherein at position (a) Formula (IV) is attached to the nitrogen atom and the (CH₂)ₚ group is attached to R⁸, and wherein R¹¹ is selected from hydrogen, optionally substituted C₁₋₆alkyl or N(R²³R²⁴),
where R²³ and R²⁴ are independently selected from: hydrogen, hydroxy, optionally substituted C₁₋₆alkyl, optionally substituted aryl, optionally substituted arylC₁₋₆alkyl, an optionally substituted carbocyclic ring of 3-7 atoms, optionally substituted heterocyclyl, optionally substituted heterocyclylC₁₋₆alkyl or R²³ and R²⁴ taken together can form an optionally substituted ring of 3-9 atoms, wherein the optional substituents for any optionally substituted group R²³, R²⁴ and C₁₋₆alkyl groups R¹¹ are selected from R¹² and 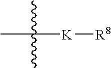
where K and R⁸ are as defined herein;
(iii) a group independently selected from: optionally substituted C₁₋₆alkylene, optionally substituted C₃₋₇cycloalkyl, optionally substituted C₃₋₆alkenylene, optionally substituted C₃₋₆alkynyl, (C₁₋₅alkyl)ₐₐ-S(Oₙ)—(C₁₋₅alkyl)_{bb}-, —(C₁₋₅alkyl)ₐₐ-O-(C₁₋₅alkyl)_{bb}-, —(C₁₋₅alkyl)ₐₐ-C(O)—(C₁₋₅alkyl)_{bb}- or (C₁₋₅alkyl)ₐₐ-N(R^{14a})-(C₁₋₅alkyl)_{bb}, or —(C₁₋₅alkyl)ₐₐ-C(O)NR^{14a}—(C₁₋₅alkyl)_{bb}-
wherein R^{14a} is a group R¹⁴ as defined above, or R^{14a} and the (C₁₋₅alkyl)ₐₐ or (C₁₋₅alkyl)_{bb} chain can be joined to form a heterocyclic ring, wherein aa and bb are independently 0 or 1 and the combined length of (C₁₋₅alkyl)ₐₐ and (C₁₋₅alkyl)_{bb} is less than or equal to C₅alkyl and wherein the optional substituents are independently selected from R¹²;
or the group —B—R⁸ represents a group of Formula (V) 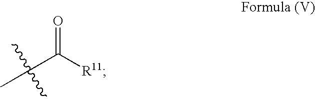
or the group 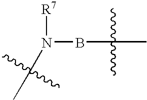
together forms an optionally substituted heterocyclic ring containing 4-7 carbons atoms, wherein the optional substituents are selected from 1 or 2 substituents independently selected from R¹² and R¹³;
or the group 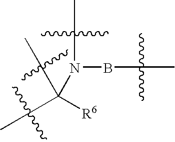
forms a heterocyclic ring containing 3-7 carbon atoms and one or more heteroatoms;
J is a group of the formula: —(CH₂)ₛ-L-(CH₂)ₛ— or —(CH₂)ₛ—C(O)—(CH₂)ₛ-L-(CH₂)ₛ— wherein when s is greater than 0, the alkylene group is optionally substituted by 1 to 2 group selected from R¹²,
or the group 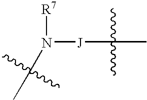
together forms an optionally substituted heterocyclic ring containing 4-7 carbons atoms, wherein the optional substituents are selected from 1 or 2 substituents independently selected from R¹² and R¹³;
K is selected from: a direct bond, —(CR²¹R²²)ₛ₁—, —(CR²¹R²²)ₛ₁—O—(CR²¹R²²)ₛ₂—, —(CR²¹R²²)ₛ₁—C(O—(CR²¹R²²)ₛ₂—, —(CR²¹R²²)ₛ₁—S(O)ₙ—(CR²¹R²²)ₛ₂—, —(CR²¹R²²)ₛ₁—N(R¹⁷)—(CR²¹R²²)ₛ₂—, —(CR²¹R²²)ₛ₁—C(O)N(R¹⁷)—(CR²¹R²²)ₛ₂—, —(CR²¹R²²)ₛ₁—N(R¹⁷)C(O)—(CR²¹R²²)ₛ₂—, —(CR²¹R²²)ₛ₁—N(R¹⁷)C(O)N(R¹⁷)—(CR²¹R²²)ₛ₂—, —(CR²¹R²²)ₛ₁—OC(O)—(CR²¹R²²)ₛ₂—, —(CR²¹R²²)ₛ₁—C(O)O—(CR²¹R²²)ₛ₂—, —(CR²¹R²²)ₛ₁—N(R¹⁷)C(O)O—(CR²¹R²²)ₛ₂, —(CR²¹R²²)ₛ₁—OC(O)N(R¹⁷)—(CR²¹R²²)ₛ₂—, —(CR²¹R²²)ₛ₁—OS(Oₙ)—(CR²¹R²²)ₛ₂, or —(CR²¹R²²)ₛ₁—S(Oₙ)—O—(CR²¹R²²)ₛ₂—, —(C²¹R²²)ₛ₁—S(O)₂N(R¹⁷)—(CR²¹R²²)ₛ₂— or —(CR²¹R²²)ₛ₁—N(R¹⁷)S(O)₂—(CR²¹R²²)ₛ₂—; wherein each R²¹ and R²² group is independently selected from hydrogen, hydroxy or optionally substituted C₁₋₄alkyl, wherein the optional substituent is a group ZR³⁰ where Z is oxygen or a group S(O)ₙ where n is as described above, and R³⁰ is hydrogen or C₁₋₄alkyl;
L is selected from optionally substituted aryl or optionally substituted heterocyclyl;
n is an integer from 0 to 2;
p is an integer from 0 to 4;
s, s1 and s2 are independently selected from an integer from 0 to 4, and
s1+s2 is less than or equal to 4;
with the proviso that the compound must contain at least one of the following groups:
(i) R³ is a group of formula (IIc) or (IId) wherein J is a group of the formula: —(CH₂)ₛ—C(O)—(CH₂)ₛ-L-(CH₂)ₛ-, and/or
(ii) R² is selected from hydrogen or optionally substituted C₁₋₆alkyl, and/or
(iii) at least one group R⁶ or R^{6a} is selected from C₁₋₆alkoxy, N-C₁₋₆alkylamino and N,N-diC₁₋₆alkylamino, and/or
(iv) R⁵ is a group of formula III-k, III-I, III-o or III-p, and/or
(v) R⁸ is selected from substituted C₃₋₇cycloalkyl, substituted aryl or substituted arylC₁₋₆alkyl, wherein a substituent is a group R¹³; or R⁸ is a heterocyclyl or heterocyclylC₁₋₆alkyl each of which is substituted by a group R¹³ and optionally by up to 3 further substituents independently selected from R¹² or R¹³; and/or
(vi) both of R²¹ and R²² within a —(CR²¹R²²)ₛ₁— or —(CR²¹R²²)ₛ₂— are C₁₋₄alkyl; or
(vii) at least one of R²¹ or R²² within a —(CR²¹R²²)ₛ₁— or —(CR²¹R²²)ₛ₂— is a C₁₋₄alkyl which is optionally substituted by a group ZR³⁰,
or a salt, solvate or pro-drug thereof.

2. A compound according to claim 1 wherein the compound is a compound of formula (I) as defined therein, with the proviso that the compound contains at least one of the following groups:
(i) R³ is a group of formula (IIc) or (IId) wherein J is a group of the formula:
—(CH₂)ₛ—C(O)—(CH₂)ₛ-L-(CH₂)ₛ—, and/or
(ii) R² is selected from hydrogen or optionally substituted C₁₋₆alkyl, and/or
(iii) at least one group R⁶ or R^{6a} is selected from C₁₋₆alkoxy, N-C₁₋₆alkylamino and N,N-diC₁₋₆alkylamino, and/or
(iv) R⁵ is a group of formula III-k, III-l, III-o or III-p, and/or
(vi) a CH₂ group within a —(CH₂)ₛ₁— or —(CH₂)ₛ₂— is di-substituted with C₁₋₄alkyl; and/or
(vii) a —(CH₂)ₛ₁— or —(CH₂)ₛ₂— moiety is a branched C₁₋₄alkyl optionally substituted by a —S(O)ₙR³⁰.

3. A compound according to claim 2 wherein the compound of formula (I) includes a least one of the following groups:
(i) R³ is a group of formula (IIc) or (IId) wherein J is a group of the formula: —CH₂)ₛ—C(O)—(CH₂)ₛ-L-(CH₂)ₛ—, and/or
(iii) at least one group R⁶ or R^{6a} is selected from C₁₋₆alkoxy, N-C₁₋₆alkylamino and N,N-diC₁₋₆alkylamino, and/or
(iv) R⁵ is a group of formula III-k, III-l, III-o or III-p, and/or
(vi) a CH₂ group within a —(CH₂)ₛ₁— or —(CH₂)ₛ₂— is di-substituted with C₁₋₄alkyl; and/or
(vii) a —(CH₂)ₛ₁— or —(CH₂)ₛ₂— moiety is a branched C₁₋₄alkyl optionally substituted by a —S(O)ₙR³⁰.

4. A compound according to claim 1 of formula (Id′) 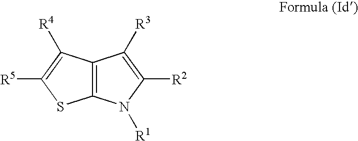
wherein:
R³ is selected from a group of Formula (IIc) or Formula (IId): 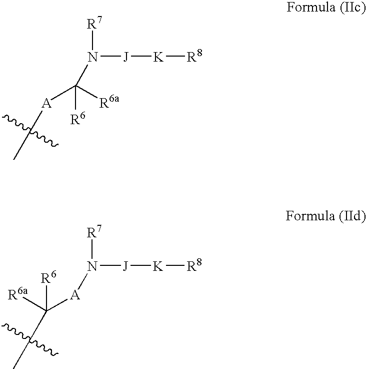
J is a group of the formula: —(CH₂)ₛ—C(O)—(CH₂)ₛ-L-(CH₂)ₛ wherein when s is greater than 0, the alkylene group is optionally substituted by 1 to 2 groups selected from R¹²,
and A, K, L, R¹, R², R⁴, R⁵ R⁶, R^{6a}, R⁸, and R¹² are as defined above for a compound of Formula (I);
or a salt, solvate or pro-drug thereof.

5. A compound according to claim 4 wherein L is azetidinyl, and each s group is 0.

6. A compound according to claim 1 wherein, in formula (I), at least one of R⁶ or R^{6a} is selected from C₁₋₆alkoxy, N-C₁₋₆alkylamino or N,N-diC₁₋₆alkylamino.

7. A compound according to claim 6 wherein said one of R⁶ or R^{6a} is C₁₋₆alkoxy.

8. A compound of formula (Ie) which is a compound of formula (I) as claimed in claim 1 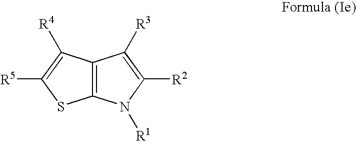
wherein:
R³ is selected from a group of Formula (IIc) or Formula (IId): 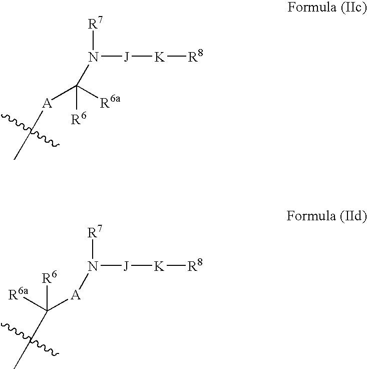
wherein
K is selected from: —(CR²¹R²²)ₛ₁—, —(CR²¹R²²)ₛ₁—O—(CR²¹R²²)ₛ₂—, —(CR²¹R²²)ₛ₁—C(O—(CR²¹R²²)ₛ₂—, —(CR²¹R²²)ₛ₁—S(O)ₙ—(CR²¹R²²)ₛ₂—, —(CR²¹R²²)ₛ₁—N(R¹⁷)—(CR²¹R²²)ₛ₂—, —(CR²¹R²²)ₛ₁—C(O)N(R¹⁷)—(CR²¹R²²)ₛ₂—, —(CR²¹R²²)ₛ₁—N(R ¹⁷)C(O)—(CR²¹R²²)ₛ₂—, —(CR²¹R²²)ₛ₁—N(R¹⁷)C(O)N(R¹⁷)—(CR²¹R²²)ₛ₂—, —(CR²¹R²²)ₛ₁—OC(O)—(CR²¹R²²)ₛ₂—, —(CR²¹R²²)ₛ₁—C(O)O—(CR²¹R²²)ₛ₂—, —(CR²¹R²²)ₛ₁—N(R¹⁷)C(O)O—(CR²¹R²²)ₛ₂, —(CR²¹R²²)ₛ₁—OC(O)N(R¹⁷)—(CR²¹R²²)ₛ₂—, —(CR²¹R²²)ₛ₁—OS(Oₙ)—(CR²¹R²²)ₛ₂, or —(CR²¹R²²)ₛ₁—S(Oₙ)—O—(CR²¹R²²)ₛ₂—, —(CR²¹R²²)ₛ₁—S(O)₂N(R¹⁷)—(CR²¹R²²)ₛ₂— or —(CR²¹R²²)ₛ₁—N(R¹⁷)S(O)₂—(CR²¹R²²)ₛ₂—; where R¹⁷, n, s1 and s2 are as defined in claim 1, and each R²¹ and R²² group is independently selected from hydrogen, hydroxy or optionally substituted C₁₋₄alkyl, wherein the optional substituent is a group ZR³⁰ where Z is oxygen or a group S(O)ₙ where n is as described above, and R³⁰ is hydrogen or C₁₋₄alkyl, provided that at least one group R²¹ or R²² is a C₁₋₄alkyl substituted by a group ZR³⁰
and A, J, L, R¹, R², R⁴, R⁵ R⁶, R^{6a}, R⁸, and R¹² are as defined in claim 1;
or a or a salt, solvate or pro-drug thereof.

9. A compound of formula (If) which is a compound of formula (I) as claimed in claim 1 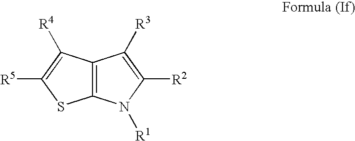
wherein:
R³ is selected from a group of Formula (IIc) or Formula (IId): 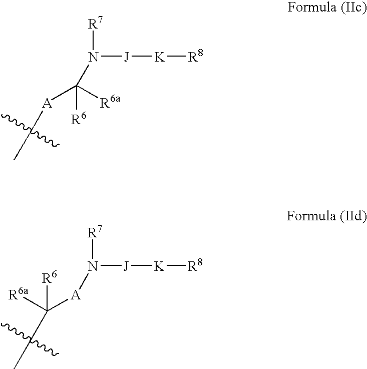
wherein
K is selected from: —(CR²¹R²²)ₛ₁—, —(CR²¹R²²)ₛ₁—O—(CR²¹R²²)ₛ₂—, —(CR²¹R²²)ₛ₁—C(O—(CR²¹R²²)ₛ₂—, —(CR²¹R²²)ₛ₁—S(O)ₙ—(CR²¹R²²)ₛ₂—, —(CR²¹R²²)ₛ₁—N(R¹⁷)—(CR²¹R²²)ₛ₂—, —(CR²¹R²²)ₛ₁—C(O)N(R¹⁷)—(CR²¹R²²)ₛ₂—, —(CR²¹R²²)ₛ₁—N(R¹⁷)C(O)—(CR²¹R²²)ₛ₂—, —(CR²¹R²²)ₛ₁—N(R¹⁷)C(O)N (R¹⁷)—(CR²¹R²²)ₛ₂—, —(CR²¹R²²)ₛ₁—OC(O)—(CR²¹R²²)ₛ₂—, —(CR²¹R²²)ₛ₁—C(O)O—(CR²¹R²²)ₛ₂—, —(CR²¹R²²)ₛ₁—N(R¹⁷)C(O)O—(CR²¹R²²)ₛ₂, —(CR²¹R²²)ₛ₁—OC(O)N(R¹⁷)—(CR²¹R²²)ₛ₂—, —(CR²¹R²²)ₛ₁—OS(Oₙ)—(CR²¹R²²)ₛ₂, or —(CR²¹R²²)ₛ₁—S(Oₙ)—O—(CR²¹R²²)ₛ₂—, —(CR²¹R²²)ₛ₁—S(O)₂N(R¹⁷)—(CR²¹R²²)ₛ₂— or —(CR²¹R²²)ₛ₁—N(R¹⁷)S(O)₂—(CR²¹R²²)ₛ₂—; where R¹⁷, n, s1 and s2 are as defined in claim 1, and each R²¹ and R²² group is independently selected from hydrogen, hydroxy or optionally substituted C₁₋₄alkyl, wherein the optional substituent is a group ZR³⁰ where Z is oxygen or a group S(O)ₙ where n is as described above, and R³⁰ is hydrogen or C₁₋₄alkyl, provided that both group R²¹ and R²² within the same —(CR²¹R²²)ₛ₁— or —(CR²¹R²²)ₛ₂— is a C₁₋₄alkyl group;
and A, J, L, R¹, R², R⁴, R⁵ R⁶, R^{6a}, R⁸, and R¹² are as defined in claim 1;
or a or a salt, solvate or pro-drug thereof.

10. A compound selected from:
2-(1,1-dimethyl-2-oxo-2-azabicyclo[2.2.1]heptan-7-ylethyl)-4-[1S-methyl-2-(1-{3-hydroxybenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{3-cyanobenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{3-nitrobenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl )-4-[1-methoxy-2-(4-{pyrrolidin-1-ylcarbonylmethyl}-piperazin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl )-4-[1-methoxy-2-(4-{morpholinocarbonyl}-piperazin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1-methoxy-2-(4-{4-methoxypiperidin-1-ylcarbonyl}-piperazin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[2-(4-{1-carbamoyl-3-methylthio-propylaminocarbonyl}-piperidin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[2-(4-{1-carbamoyl-2-methylthio-ethylpropylaminocarbonyl}-piperidin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[2-(4-{morpholinocarbonyl-1,1-dimethylmethylene}-piperidin-1-yl)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{thien-2-ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{benzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{pyrid-3-ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{benzodioxol-5-ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{3-hydroxybenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{pyrrol-2ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{4-fluorobenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{3-chlorobenzyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{thien-3-ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{furan-3-ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{cyclohexylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[1S-methyl-2-(1-{thiazol-2-ylmethyl}-azetidin-3-ylcarbonylamino)ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[2-(4-{1-carbamoyl-2hydroxy-ethylaminocarbonyl}-piperidin-1-yl )ethyl]-5-(3,5-dimethylphenyl)-6H-thieno[2,3-b]pyrrole;
2-(1,1-dimethyl-2-oxo-2-azabicyclo[2.2.1]heptan-7-ylethyl)-4-[2-(4-{1-carbamoyl-2hydroxy-propylaminocarbonyl}-piperidin-1-yl)ethyl]-5-(3,5-dimethylphenyl)-6H-2-(1,1-dimethyl-2-oxo-2-azabicyclo[2.2.1]heptan-7-ylethyl)-4-[2-(4-{1-carbamoyl-3-hydroxy-propylaminocarbonyl}-piperidin-1-yl)ethyl]-5-(3,5-dimethylphenyl)-6H-thieno[2,3-b]pyrrole; and
2-(1,1-dimethyl-2-oxo-2-azabicyclo[2.2.1]heptan-7-ylethyl)-4-[2-(4-{3-hydroxypyrrolidin-1-ylcarbonyl-1,1-dimethylmethylene}-piperidin-1-yl)ethyl]-5-(3,5-dimethylphenyl)-6H-thieno[2,3-b]pyrrole;
or a salt, pro-drug or solvate thereof.

11. A compound selected from
2-(1,1-dimethyl-2-oxo-2-azabicyclo[2.2.1]heptan-7-ylethyl)-4-[2-(4-{1-carbamoyl-3-methyl-butylaminocarbonyl}-piperidin-1-yl)ethyl]-5-(3,5-dimethylphenyl)-6h-thieno[2,3-b]pyrrole;
2-(1,1-dimethyl-2-oxo-2-azabicyclo[2.2.1]heptan-7-ylethyl)-4-[2-(4-{1-N-methylcarbamoyl-3-methyl-butylaminocarbonyl}-piperidin-1-yl)ethyl]-5-(3,5-dimethylphenyl)-6h-thieno[2,3-b]pyrrole;
2-(1,1-dimethyl-2-oxo-2-azabicyclo[2.2.1]heptan-7-ylethyl)-4-[2-(4-{1-carbamoyl-but-1-ylaminocarbonyl}-piperidin-1-yl)ethyl]-5-(3,5-dimethylphenyl)-6h-thieno[2,3-b]pyrrole;
2-(1,1-dimethyl-2-oxo-2-azabicyclo[2.2.1]heptan-7-ylethyl)-4-[2-(4-{1-carbamoyl-eth-1-ylaminocarbonyl}-piperidin-1-yl)ethyl]-5-(3,5-dimethylphenyl)-6h-thieno[2,3-b]pyrrole;
2-(1,1-dimethyl-2-oxo-2-azabicyclo[2.2.1]heptan-7-ylethyl)-4-[2-(4-{1-carbamoyl-prop-1-ylaminocarbonyl}-piperidin-1-yl)ethyl]-5-(3,5-dimethylphenyl)-6h-thieno[2,3-b]pyrrole; and
2-(1,1-dimethyl-2-oxo-2-azabicyclo[2.2.1]heptan-7-ylethyl)-4-[2-(4-{1-carbamoyl-eth-1-ylaminocarbonyl}-piperidin-1-yl)ethyl]-5-(3,5-dimethylphenyl)-6h-thieno[2,3-b]pyrrole.

12. A process for preparing a compound according to claim 1, said process comprising a step selected from (a) to (g):—
(a) Reaction of a compound of formula XXXII with a compound of formula H—R^{3′} 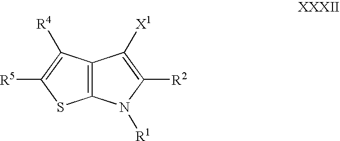
wherein X¹ is selected from: 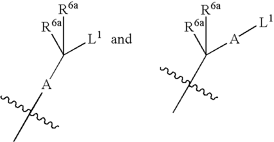
L¹ is a displaceable group; and
H—R^{3′} is selected from: 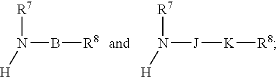
where R¹, R², R⁴, R⁵, R⁷, R⁸, B, J, K, A, R⁶ and R^{6a} are as defined above;
(b) Reaction of a compound of formula XXXIII with a compound of formula L²-R^{3″} 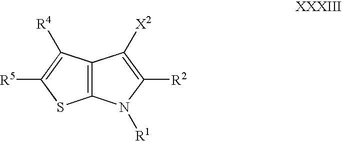
wherein X² is selected from: 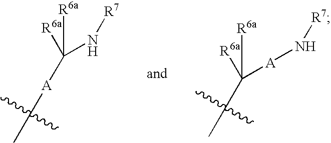
L² is a displaceable group and R⁷ is selected from the definition of R⁷, and
L²-R^{3″} is selected from: L²-B—R⁸ and L²-J-K—R⁸,
where R¹, R², R⁴, R⁵, R⁷, R⁸, B, J, K, A, R⁶ and R^{6a} are as defined above;
(c) for compounds of Formula (I) wherein R⁷ is other than part of a heterocyclic ring or hydrogen, reaction of a compound of Formula (I) wherein R⁷ is hydrogen with a group of formula L³-R^{7a}, wherein R^{7a} is as defined above for R⁷ with the exclusion of hydrogen and L³is a displaceable group;
(d) for compounds of Formula (I) wherein R³ is a group of Formula (IIc) or (IId) and
the group 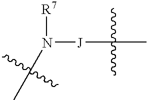
together forms an optionally substituted nitrogen-containing heterocyclic ring containing 4-7 carbons atoms, reaction of a compound of Formula XXXIVa or XXXIVb, with a compound of Formula L⁶-K—R⁸, wherein L⁶ is a displaceable group 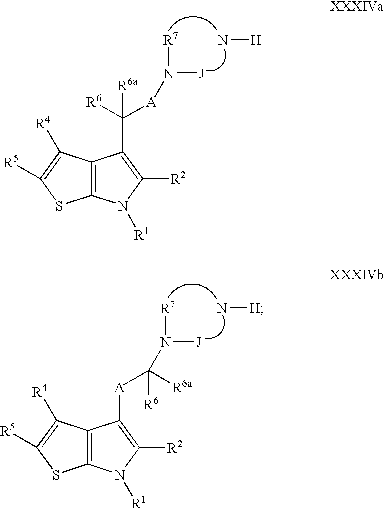
where R¹, R², R⁴, R⁵, R⁷, R⁸, J, K, A, R⁶ and R^{6a} are as defined above;
(e) for compounds of Formula (I) wherein R³ is a group of Formula (IIc) or (IId), reaction of a compound of Formula XXXVa or XXXVb, with a compound of Formula L⁷-K″—R⁸, wherein L⁷ is a displaceable group, and wherein the groups K′ and K″ comprise groups which when reacted together form K, 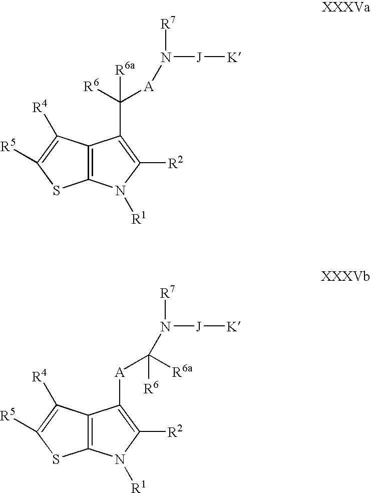
where R¹, R², R⁴, R⁵, R⁷, R⁸, J, K, A, R⁶ and R^{6a} are as defined above;
(f) reaction of a compound of Formula XXXVI with an electrophilic compound of the formula L⁸-R³, wherein L⁸ is a displaceable group 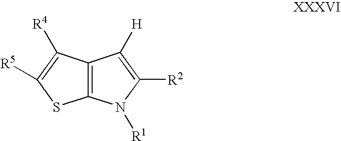
where R¹, R², R³, R⁴ and R⁵ are as defined above;
(g) reaction of a compound of Formula XXXVII with a compound of the formula L¹⁰-R² , wherein L⁹ is a leaving group and L¹⁰ is an activating group or L⁹ is an activating group and L¹⁰ is a leaving group 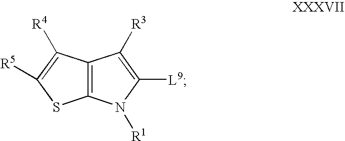
where R¹, R², R³, R⁴ and R⁵ are as defined above;
and thereafter if necessary, carrying out one or more of the following steps:
i) converting a compound of the Formula (I) into another compound of the Formula (I);
ii) removing any protecting groups; or
iii) forming a salt, pro-drug or solvate.

13. A pharmaceutical formulation comprising a compound according to claim 1, or salt, pro-drug or solvate thereof, and a pharmaceutically acceptable diluent or carrier.

14. A method of antagonising gonadotropin releasing hormone activity in a patient, comprising administering a compound according to claim 1, or salt, pro-drug or solvate thereof, to a patient.

15-16. (canceled)
